(19) United States
(12) Patent Application Publication
(10) Pub. No.: US 2024/0399154 A1
(43) Pub. Date: Dec. 5, 2024

(54) ELECTRIC FIELD THERAPY VIA IMPLANTABLE ELECTRODE

(71) Applicant: Medtronic, Inc, Minneapolis, MN (US)

(72) Inventors: Benjamin Kevin Hendrick, Phoenix, AZ (US); Steven M. Goetz, North Oaks, MN (US); David A. Simon, Boulder, CO (US); Maneesh Shrivastav, Blaine, MN (US); Leslie Hiemenz Holton, Pine, CO (US); Xuan K. Wei, Minnetonka, MN (US); David J. Miller, Austin, TX (US); Ryan B. Sefkow, Plymouth, MN (US); Phillip C. Falkner, Minneapolis, MN (US); Meredith S. Seaborn, Minneapolis, MN (US); Richard T. Stone, Roseville, MN (US); Robert L. Olson, White Bear Lake, MN (US); Scott D DeFoe, Andover, MN (US)

(21) Appl. No.: 18/688,210

(22) PCT Filed: Sep. 8, 2022
(86) PCT No.: PCT/US2022/042940

§ 371 Date: Feb. 29, 2024

Related U.S. Application Data
(60) Provisional application No. 63/241,924, filed on Sep. 8, 2021.
(60) Provisional application No. 63/316,243, filed on Mar. 3, 2022.

(51) Int. Cl. A61N 1/36; A61N 1/05; A61N 1/372; A61N 1/378; A61N 2/00; A61N 2/02

(52) U.S. Cl. CPC A61N 1/36185; A61N 1/0529; A61N 1/36002; A61N 1/36125; A61N 1/3615; A61N 1/37247; A61N 1/378; A61N 2/004; A61N 2/02

(57) ABSTRACT

Devices, systems, and techniques are disclosed for planning, updating, and delivering electric field therapy. In one example, a system comprises processing circuitry configured to receive a request to deliver alternating electric field (AEF) therapy and determine therapy parameter values that define the AEF therapy, wherein the AEF therapy comprises delivery of a first electric field and a second electric field. The processing circuitry may also be configured to control an implantable medical device to deliver the first electric field from a first electrode combination of implanted electrodes and control the implantable medical device to deliver, alternating with the first electric field, the second electric field from a second electrode combination of implanted electrodes different than the first electrode combination.

This application is a PCT application that claims the benefit of and priority to U.S. Provisional Patent Application No. 63/241,924, filed Sep. 8, 2021, and U.S. Provisional Patent Application No. 63/316,243, filed Mar. 3, 2022, the entire contents of which are incorporated herein by reference.

### TECHNICAL FIELD

This disclosure generally relates to electrical stimulation therapy using alternating electric fields.

### BACKGROUND

Alternating electric field (AEF) therapy, is a type of electric field therapy which uses low-intensity electrical fields to treat brain tumors; glioblastoma in particular. Conventional cancer treatments include chemotherapy and radiation, which are associated with treatment-related toxicity and high rates of tumor recurrence. AEF uses an alternating electric field to disrupt cell division in cancer cells, thereby inhibiting cellular replication and initiating apoptosis (cell death). AEF therapy is typically delivered via electrodes located external to the patient.

### SUMMARY

In general, the disclosure describes devices, systems, and techniques related to planning, delivering, and adjusting electrical field therapy. Electric field therapy may include modulated electrical field therapy which may include types of electrical field modulation, such as alternating electrical field (AEF) therapy which is discussed herein as one example. For example, an implantable medical device (IMD) may be coupled to one or more leads carrying an array of electrodes. In one example, the IMD may be coupled to a plurality of leads, wherein each lead may include a non-conductive cap coupled to a post, and where the post carries one or more electrodes along the axial length the post. These cap and post leads may be referred to as “tack electrodes” or “tack leads.” The IMD may alternate delivery of electrical fields from respective different electrode combinations utilizing some or all of the implanted electrodes. The IMD may generate these electrical fields using a constant frequency, a different frequency for the alternating electrical fields, or a frequency for the alternating electrical fields that changes over time. The IMD may also cycle the AEF therapy on and off according to a predetermined schedule or in response to one or more trigger events. The AEF therapy may be used for various reasons, such as reducing or preventing the growth of tumor cells, such as glioblastomas, or the reduction in growth or proliferation of non-tumorous cells within the body.

In some examples, the ID or a different computing device may model the anatomy of the patient that will receive the AEF therapy. For example, the computing device may receive imaging data from one or more imaging modalities (e.g., magnetic resonance imaging (MRI), computed tomography (CT), etc.) and identify locations of various tissues and/or structures, such as a resection bed from which a tumor was removed. The computing device may generate a patient specific model of anatomy, such as brain tissue, based on the imaging data. The computing device may also predict electrical field strengths for AEF therapy using the model and recommend stimulation parameters (e.g., electrode combinations, amplitudes, frequencies, electrode implant locations, etc.) that define the AEF therapy based on the predictions. In some examples, the computing device may update the predictions based on sensed data obtained after delivery of the AEF therapy.

In one example, a system includes processing circuitry configured to receive a request to deliver alternating electric field (AEF) therapy, determine therapy parameter values that define the AEF therapy, wherein the AEF therapy comprises delivery of a first electric field and a second electric field, control an implantable medical device to deliver the first electric field from a first electrode combination of implanted electrodes, and control the implantable medical device to deliver, alternating with the first electric field, the second electric field from a second electrode combination of implanted electrodes different than the first electrode combination.

In another example, a system includes processing circuitry configured to receive a request to deliver electric field therapy; determine therapy parameter values that define the electric field therapy, wherein the electric field therapy comprises delivery of a first electric field and a second electric field, control an implantable medical device to deliver the first electric field from a first electrode combination of implanted electrodes, and control the implantable medical device to deliver, alternating with the first electric field, the second electric field from a second electrode combination of implanted electrodes different than the first electrode combination.

In another example, a method includes receiving, by processing circuitry, a request to deliver electric field therapy, determining therapy parameter values that define the electric field therapy, wherein the electric field therapy comprises delivery of a first electric field and a second electric field, controlling, by processing circuitry, an implantable medical device to deliver the first electric field from a first electrode combination of implanted electrodes, and controlling, by the processing circuitry, the implantable medical device to deliver, alternating with the first electric field, the second electric field from a second electrode combination of implanted electrodes different than the first electrode combination.

In another example, a computer readable medium includes instructions that, when executed by processing circuitry, causes the processing circuitry to receive a request to deliver electric field therapy, determine therapy parameter values that define the electric field therapy, wherein the electric field therapy comprises delivery of a first electric field and a second electric field, control an implantable medical device to deliver the first electric field from a first electrode combination of implanted electrodes, and control the implantable medical device to deliver, alternating with the first electric field, the second electric field from a second electrode combination of implanted electrodes different than the first electrode combination.

The details of one or more examples of the techniques of this disclosure are set forth in the accompanying drawings and the description below. Other features, objects, and advantages of the techniques will be apparent from the description and drawings, and from the claims.

### BRIEF DESCRIPTION OF DRAWINGS

FIG. 1 is a conceptual diagram illustrating an example system that includes an implantable medical device (IMD) configured to deliver alternating electric field (AEF) therapy to a patient according to an example of the techniques of the disclosure.

FIG. 2 is a block diagram of the example IMD of FIG. 1 for delivering AEF therapy according to an example of the techniques of the disclosure.

FIG. 3 is a block diagram of the external programmer of FIG. 1 for controlling delivery of AEF therapy according to an example of the techniques of the disclosure.

FIG. 4 is a block diagram illustrating an example system that includes an external device, such as a server, and one or more computing devices that are coupled to an implantable medical device and external programmer shown in FIG. 1 via a network.

FIGS. 5A and 5B are conceptual diagrams of example leads with respective electrodes carried by the lead.

FIGS. 5C, 5D, 5E, and 5F are conceptual diagrams of example electrodes disposed around a perimeter of a lead at a particular longitudinal location.

FIG. 6 is a flowchart illustrating an example technique for delivering AEF therapy to a patient.

FIG. 7 is a flowchart illustrating an example technique for generating a model of anatomy for a patient for AEF therapy planning.

FIG. 8 is a flowchart illustrating an example technique for predicting electrical field strength for AEF therapy.

FIG. 9 is a flowchart illustrating an example technique for determining stimulation parameters for AEF therapy based on user input identifying target tissue.

FIG. 10 is a conceptual diagram of an example three-dimensional user interface for programming AEF therapy.

FIG. 11 is a flowchart illustrating an example technique for determining stimulation parameters for AEF therapy based on histological data from tumor tissue.

FIG. 12 is a flowchart illustrating an example technique for identifying target tissue for AEF therapy based on water content determined from imaging data.

FIG. 13 is a flowchart illustrating an example technique for identifying target tissue based on impedance tomography data.

FIG. 14 is a flowchart illustrating an example technique for generating a map of AEF dosimetry for a patient.

FIG. 15 is a flowchart illustrating an example technique for sweeping through different frequencies for AEF therapy.

FIG. 16 is a conceptual diagram of an example three-dimensional user interface for planning implantation of electrodes for AEF therapy.

FIG. 17 is a flowchart illustrating an example technique for adjusting stimulation parameters for AEF therapy based on sensed electrical signals.

FIG. 18A is a flowchart illustrating an example technique for switching polarity of electrodes for AEF therapy.

FIG. 18B is a conceptual drawing illustrating an example progression of polarity switching for AEF therapy.

FIG. 19A is a flowchart illustrating an example technique for switching polarity between electrode pairs of a cube configuration for AEF therapy.

FIG. 19B is a conceptual drawing illustrating an example progression of paired electrode selection using cube configurations for AEF therapy.

FIG. 20 is a flowchart illustrating an example technique for displaying user selectable electrode configurations for AEF therapy.

FIG. 21 is a conceptual diagram illustrating an example implantable medical device for delivering AEF therapy.

FIG. 22 is a conceptual diagram illustrating example implantable medical devices disposed within burr holes for delivering AEF therapy.

FIG. 23 is a conceptual diagram of an example three-dimensional user interface for planning implantation of electrode arrays for AEF therapy.

FIG. 24 is a conceptual diagram illustrating example subcutaneous electrodes for use in delivering AEF therapy.

FIG. 25 is a conceptual diagram illustrating example cutaneous electrodes and an external medical device for use in combination with an IMD for delivering AEF therapy.

FIG. 26 is a flowchart illustrating an example technique for scheduling electrical field sensing based on patient activity.

FIG. 27 is a conceptual diagram illustrating example implantable coils for delivering alternating magnetic field (AMF) therapy.

FIG. 28 is a flowchart illustrating an example technique for delivering AMF therapy using multiple implantable coils.

FIG. 29 is a flowchart illustrating an example technique for cycling AEF therapy based on sensed temperatures of patient tissue.

FIG. 30 is a flowchart illustrating an example technique for synchronizing AEF therapy to cell cycle phases.

FIG. 31 is a flowchart illustrating an example technique for cycling through different frequencies for AEF therapy.

FIG. 32 is a flowchart illustrating an example technique for delivering AEF therapy that includes different frequencies from different electrode combinations.

FIG. 33 is a flowchart illustrating an example technique for adjusting the frequency of AEF therapy in response to detecting a trigger event.

FIG. 34 is a flowchart illustrating an example technique for adjusting the frequency of AEF therapy in response to tissue changes indicated by image data.

FIG. 35 is a flowchart illustrating an example technique for delivering AEF therapy to activate an exogenous agent injected into the patient.

FIG. 36 is a flowchart illustrating an example technique for delivering a voltage bolus configured to irreversible electroporation of cells in a target tissue.

FIG. 37 is a conceptual diagram of example tack electrodes configured to be implanted within the surface of a resection cavity of a patient.

FIG. 38 is a flowchart illustrating an example technique for implanting multiple tack electrodes within a resection cavity of a patient.

FIG. 39 is a flowchart illustrating an example technique for planning tumor removal and electric field therapy.

FIG. 40 is a flowchart illustrating an example technique for evaluating tumor removal and modifying electric field therapy.

FIG. 41 is a flowchart illustrating an example technique for determining stimulation parameter values that define electric field therapy.

FIG. 42 is a conceptual diagram of a preoperative image and estimated volume of a tumor from the image.

FIG. 43 is a conceptual diagram of a preoperative tumor volume determination.

FIG. 44 is a conceptual diagram of a preoperative tumor resection plan and subsequent implantation therapy.

FIG. 45 is a conceptual diagram of example medical leads implanted after resection of a tumor.

FIG. 46 is a conceptual diagram of example medical leads implanted due to a revised implantation plan.

FIG. 47 is a conceptual diagram of example postoperative imaging and estimated electric field coverage from implanted leads.

FIG. 48 is a conceptual diagram of example postoperative imaging and estimated electric field coverage from implanted leads including residual tumor designation.

FIG. 49A is a conceptual diagram of example leads comprising implanted within a resection bed surrounding resection void where a tumor was removed.

FIG. 49B is a conceptual diagram of an example unwrapped view of the resection bed and implanted electrodes of FIG. 49A.

FIG. 50 is a conceptual diagram of unwrapped views of potential electric fields deliverable via the implanted electrodes of FIG. 49A.

FIG. 51 is an example medical lead comprising a registration feature configured to receive a probe for determining the location of the medical lead when implanted within tissue.

FIGS. 52A and 52B are example implantation guide layers configured to facilitate implantation of leads at target locations of tissue.

FIG. 53 is a side view of example medical leads inserted through an example implantation guide layer and secured to the implantation guide layer via an adhesive layer.

### DETAILED DESCRIPTION

This disclosure describes various devices, systems, and techniques for planning and/or delivering modulated electrical field therapy (which may include the example of AEF therapy) to a patient via implanted electrodes. Alternating electric field application is a cancer treatment type with the potential to reduce treatment related toxicity. In alternating electric field application, an alternating electric field is applied to a cancerous region of the brain, which may disrupt cellular division for rapidly-dividing cancer cells. To administer alternating electric field treatment to a patient, an external system can be applied near the anatomy of interest, such as around the cranium of the patient, in order to deliver the alternating electric field to the patient. However, there are various challenges to external delivery of AEF therapy. For example, external AEF therapy requires that hair be removed from the scalp of the patient. As another example, electrical fields delivered from the external electrodes for extended periods of time required for AEF therapy may cause increased tissue heating and potential burns on the skin of the patient. In addition, external electrodes may prevent localized treatment of tumors within the brain.

As described herein, a system may deliver electric field therapy (also referred to as AEF therapy in some examples) from implanted electrodes at a location and strength specific for the patient. Electric field therapy may generally refer to therapy in which electrical fields are modulated to provide some therapeutic response. For example, alternating electric field therapy described herein includes a system that modulates electric fields by alternating between different electrode combinations, different field directions, and/or other parameters that define the electric field therapy). This internal AEF therapy may act to inhibit cellular division and/or initiate apoptosis of cancer cells at the targeted treatment location. The implanted electrodes may be selected to target tissue identified as including cancerous cells or tissue around a resection area where a previous tumor was removed. In this manner, the system may operate to deliver AEF therapy to reduce cancerous cells in the patient and/or prevent reoccurrence of cancer after resection. A computing device may be used for planning implantation of electrodes and/or selection of stimulation parameters based on imaging data obtained for the patient used to generate a model of patient tissue. In some examples, the computing device and/or IMD may adjust one or more stimulation parameters that define the AEF therapy. The system may adjust the one or more stimulation parameters based on various feedback variables, such as impedance tomography, histological analysis, patient activity, sensed temperature, and the like. In this manner, the IMD may operate in a closed-loop manner based on one or more feedback variables obtained from the patient. The AEF therapy described herein may facilitate patient-specific AEF therapy directed to specific target tissue. Using implanted electrodes may enable the system to operate over larger periods of time without impacting most patient daily activities. These and other advantages may be realized by the systems and examples described herein.

Although this disclosure is directed to delivery of AEF therapy to the brain for the purpose of treating glioblastoma, the systems, devices, and techniques described herein may similarly operate to deliver AEF therapy or similar electric-field therapies to other tissue areas and/or to treat different types of cancer. For example, a system may be implanted to treat and/or prevent cancer in the spine, pelvis, abdomen, or any other location. Some examples of target tissue may include regions of expected metastatic elements, such as lymph nodes, to reduce the spread of cells from a different tumor cite. Moreover, a human patient is described for example purposes herein, but similar systems, devices, and techniques may be used for other animals in other examples.

Electric field therapy described herein may include several different types of therapy in which different electric fields are delivered to a patient. These therapies may include modified electric field therapy, modulated electric field therapy, alternating electric field (AEF) therapy, or other therapies in which different electric fields are delivered to a patient. In some examples, these different electric fields change over time in a symmetric, non-symmetric, continuous, and/or non-continuous manner. While reference is primarily made to AEF in the examples described herein, other types of electric field therapy can be applied in the various example devices, systems, and techniques described herein.

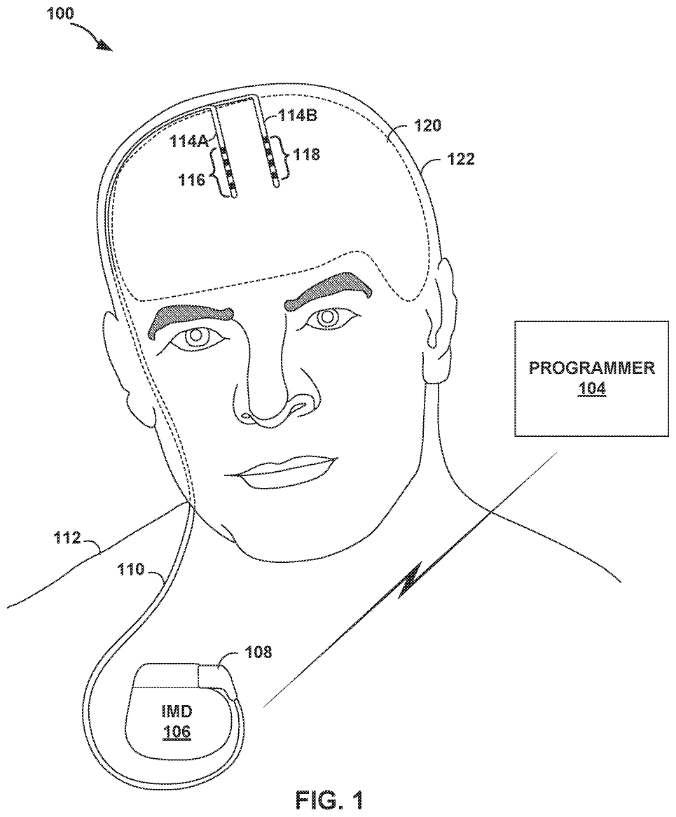

FIG. 1 is a conceptual diagram illustrating an example system 100 that includes an implantable medical device (IMD) 106 configured to deliver therapy to patient 112 according to an example of the techniques of the disclosure. This therapy may be AEF therapy or another therapy based on applied electrical fields. As shown in the example of FIG. 1, example system 100 includes medical device programmer 104, implantable medical device (IMD) 106, lead extension 110, and leads 114A and 114B with respective sets of electrodes 116, 118. In the example shown in FIG. 1, electrodes 116, 118 of leads 114A, 114B are positioned to deliver electrical stimulation to a tissue site within brain 120, such as a deep brain site under the dura mater of brain 120 of patient 112. In some examples, delivery of electric fields (e.g., electrical stimulation) to one or more regions of brain 120, such as a region that contains a tumor such as glioblastoma, or region from which a glioblastoma was resected (removed). This location where the tumor was removed, e.g., the tumor bed, may be or be part of the target tissue for AEF therapy. The tumor bed may be of various sizes, but may be between approximately 1 mm to 3 mm in diameter in some examples. Some or all of electrodes 116, 118 also may be positioned to sense neurological brain signals within brain 120 of patient 112. In some examples, some of electrodes 116, 118 may be configured to sense neurological brain signals, impedance, etc., and some or all of electrodes 116, 118 may be configured to deliver electrical stimulation to brain 120 in the form of AEF therapy. In other examples, all of electrodes 116, 118 are configured to both sense electrical signals and deliver electrical stimulation to brain 120.

IMD 106 includes a therapy module (e.g., which may include processing circuitry, signal generation circuitry or other electrical circuitry configured to perform the functions attributed to IMD 106) that includes a stimulation generator configured to generate and deliver electrical stimulation therapy (e.g., AEF therapy) to patient 112 via a subset of electrodes 116, 118 of leads 114A and 114B, respectively. The subset of electrodes 116, 118 that are used to deliver electrical stimulation to patient 112, and, in some cases, the polarity of the subset of electrodes 116, 118, may be referred to as a stimulation electrode combination. As described in further detail below, the stimulation electrode combination can be selected for a particular patient 112 and target tissue site (e.g., selected based on the patient condition or based on the determined location of a tumor or other tissue of interest). The group of electrodes 116, 118 includes at least one electrode and can include a plurality of electrodes.

In some examples, the plurality of electrodes 116 and/or 118 may have a complex electrode geometry such that two or more electrodes of the lead are located at different positions around the perimeter of the respective lead (e.g., different positions around a longitudinal axis of the lead). In this manner, electrodes at different perimeter locations may be used to generate different electrical fields. For example, anodes on a first side of a first lead and cathodes on a second side of a second lead, wherein the first sides and second side face opposing directions, may be used to generate a first electric field. The second electric field may be generated with cathodes on the second side of the first lead and anodes on the first side of the second lead. In this manner, alternating between the first and second electric fields may generate electrical current that changes the polarities of cellular components to disrupt cell division. Although two leads 14 are shown in the example of FIG. 1, a single lead, three leads, four leads, five leads, or more leads may be implanted in different examples. In any case, the combination of leads may provide an overall array of electrodes that can be programmed to deliver alternating electrical fields to a target tissue. These complex electrode geometries can also enable directional sensing that can measure the orientation of electric fields generated in tissue. For example, the system may measure electrical potentials between electrodes at different locations on a lead or between different leads to determine a gradient of electrical potentials and a gradient of the delivered electrical field. The system can then determine electric field spread and configure the electric fields and/or calibrate a predictive model of field spread based on the sensed gradient of electrical potentials.

In some examples, the neurological signals (e.g., an example type of electrical signals) sensed within brain 120 may reflect changes in electrical current produced by the sum of electrical potential differences across brain tissue. Examples of neurological brain signals include, but are not limited to, electrical signals generated from local field potentials (LFP) sensed within one or more regions of brain 120, electroencephalogram (EEG) signals, or electrocorticogram (ECoG) signals. Any of these sensed signals may be intrinsic signals generated by physiological neural activity and/or evoked signals generated in response to a delivered stimulus (e.g., a delivered electrical stimulation signal). It is noted that modulated electric field therapy (e.g., AEF therapy) may not evoke neuron propagation or affect other normal neurological function. However, the system may deliver signals intended to affect neurological processes in order to sense signals that may be indicative of physiological states or the response to modulated electric field therapy. In some examples, the system may utilize any electrode combinations to directly sense the electrical field (e.g., field strengths, field locations, or other characteristics) delivered by other electrode combinations. In this manner, the system may confirm expected electrical field strengths, adjust one or more stimulation parameters that define the electrical fields to effect target tissue (e.g., to match a desired stimulation model), and/or adjust the model of stimulation to reflect the reality of tissue characteristics. In some examples, the system may adjust the stimulation parameters defining the electrical fields to accommodate for tissue changes over time and/or lead movement within the patient after surgery or over time. The system may adjust any of these parameters in response to reviewing previously stored data and/or in real-time as sensed data is received or generated.

In some examples, the neurological brain signals that are used to select a stimulation electrode combination may be sensed within the same region of brain 120 as the target tissue site for the electrical stimulation and/or from a region different (e.g., adjacent to or outside of) than the target tissue site. The system may be configured to compute or predict the electrical field at the target tissue based on the signals sensed within the target tissue and/or at a region different than the target tissue. The specific target tissue sites and/or regions within brain 120 may be selected based on the patient condition or location, size, depth, and/or volume of a tumor or resection bed. Thus, due to these differences in target locations, in some examples, the electrodes used for delivering electrical stimulation may be different than the electrodes used for sensing neurological brain signals. In other examples, the same electrodes may be used to deliver electrical stimulation and sense brain signals. However, this configuration of using the same electrodes could require the system to switch between stimulation generation and sensing circuitry and may reduce the time the system can sense brain signals. In some examples, the system may be configured to deliver electrical signals to generate the electrical fields from the same electrode configurations (or using at least some of the same electrodes) in an at least partially interleaved basis.

Electrical stimulation generated by IMD 106 may be configured to manage a variety of disorders and conditions. In some examples, the stimulation generator of IMD 106 is configured to generate and deliver electrical stimulation pulses to patient 112 for AEF therapy via electrodes of a selected stimulation electrode combination. However, in other examples, the stimulation generator of IMD 106 may be configured to generate and deliver a continuous wave signal, e.g., a sine wave or triangle wave of specified amplitude (peak to peak) and frequency as part of the electrical fields of the AEF therapy. Generally, modulated electric field therapy (e.g., AEF therapy) may include the delivery of the continuous wave signal(s), but the waveforms may be symmetric, asymmetric, non-continuous, continuous, cycled, interleaved between different combinations, constant, or otherwise changing over time at random or predetermined sequences. In either case, a stimulation generator within IMD 106 may generate the AEF therapy according to a therapy program that is selected at that given time in therapy. In examples in which IMD 106 delivers electrical stimulation in the form of stimulation pulses, a therapy program may include a set of therapy parameter values (e.g., stimulation parameters), such as a stimulation electrode combination for delivering different electrical fields to patient 112, pulse frequency, pulse width, and a current or voltage amplitude of the pulses or continuous signals. As previously indicated, the electrode combination may indicate the specific electrodes 116, 118 that are selected to deliver stimulation signals to tissue of patient 112 and the respective polarities of the selected electrodes. IMD 106 may deliver electrical stimulation intended to contribute to a therapeutic effect. In some examples, IMD 106 may also, or alternatively, deliver electrical stimulation intended to be sensed by other electrode and/or elicit a physiological response, such as an evoked compound action potential (ECAP), that can be sensed by electrodes.

IMD 106 may be implanted within a subcutaneous pocket above the clavicle, or, alternatively, on or within cranium 122 or at any other suitable site within patient 112 such as a lower abdominal or high buttock location. Other configurations might include IMD 106 implanted at multiple locations, such as near a site of tumor occurrence and remote sites of likely tumor transmission or spread. Generally, IMD 106 is constructed of a biocompatible material that resists corrosion and degradation from bodily fluids. IMD 106 may comprise a hermetic housing to substantially enclose components, such as a processor, therapy module, and memory. Other implant locations for IMD 106 may be utilized for treatment of the brain or other tissues. Example alternative implantation sites for IMD 106 may include the lower back, shoulder, neck, abdomen, or any other location.

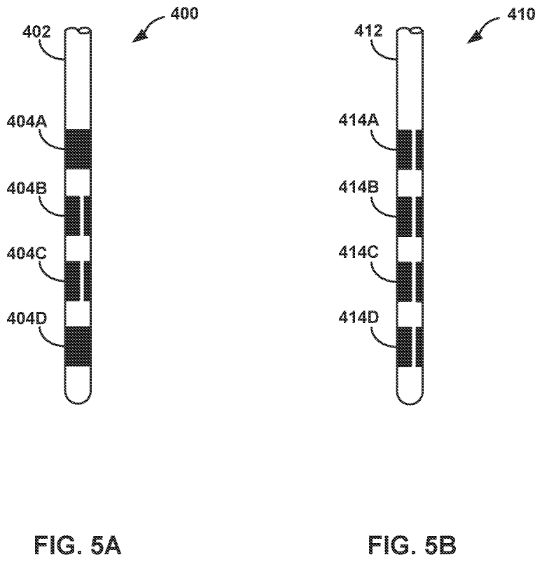

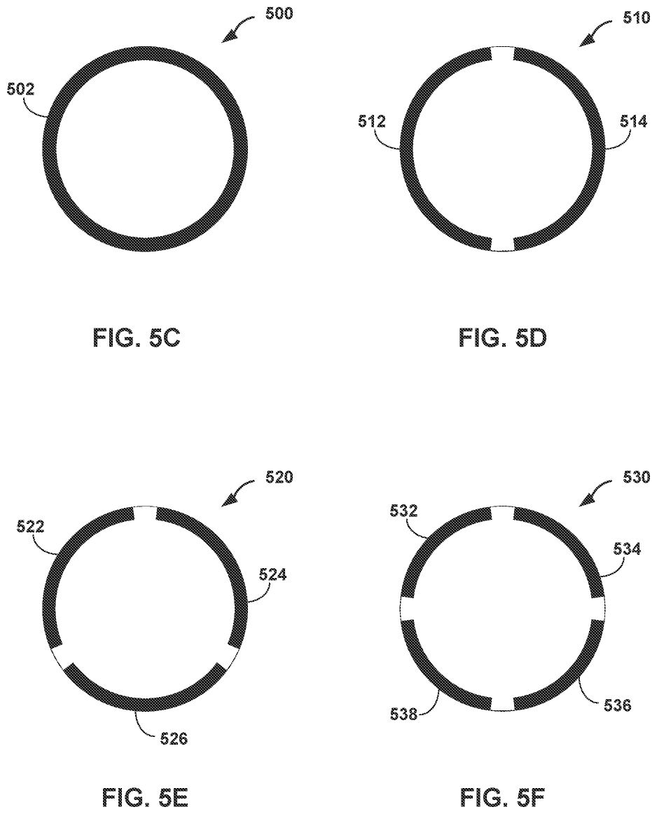

As shown in FIG. 1, implanted lead extension 110 is coupled to IMD 106 via connector 108 (also referred to as a connector block or a header of IMD 106). In the example of FIG. 1, lead extension 110 traverses from the implant site of IMD 106 and along the neck of patient 112 to cranium 122 of patient 112 to access brain 120. In the example shown in FIG. 1, leads 114A and 114B (collectively “leads 114”) are implanted within the right and left hemispheres, respectively, of patient 112 in order deliver AEF therapy to one or more regions of brain 120, which may be selected based on the patient condition or disorder controlled by therapy system 100. The specific target tissue site and the stimulation electrodes used to deliver stimulation to the target tissue site, however, may be selected, e.g., according to the locations of a tumor or resection bed and/or other sensed patient parameters. Other lead 114 and IMD 106 implant sites are contemplated. For example, IMD 106 may be implanted on or within cranium 122, in some examples. Or leads 114 may be implanted within the same hemisphere or IMD 106 may be coupled to a single lead implanted in a single hemisphere. Although leads 114 may have ring electrodes at different longitudinal positions as shown in FIG. 1, leads 114 may have electrodes disposed at different positions around the perimeter of the lead (e.g., different circumferential positions for a cylindrical shaped lead) as shown in the examples of FIGS. 5A and 5B.

Leads 114 illustrate an example lead set that include axial leads carrying ring electrodes disposed at different axial positions (or longitudinal positions). In other examples, leads may be referred to as “paddle” leads carrying planar arrays of electrodes on one side of the lead structure or a “grid” of electrodes that enable the placement of electrical elements at a variety of locations around the tissue. In addition, as described herein, complex lead array geometries may be used in which electrodes are disposed at different respective longitudinal positions and different positions around the perimeter of the lead.

Although leads 114 are shown in FIG. 1 as being coupled to a common lead extension 110, in other examples, leads 114 may be coupled to IMD 106 via separate lead extensions or directly to connector 108. Leads 114 may be positioned to deliver electrical stimulation to one or more target tissue sites within brain 120. Leads 114 may be implanted to position electrodes 116, 118 at desired locations of brain 120 through respective holes, or a common hole, in cranium 122. Leads 114 may be placed at any location within brain 120 such that electrodes 116, 118 are capable of providing electrical stimulation to target tissue sites within brain 120 during treatment. For example, electrodes 116, 118 may be surgically implanted under the dura mater of brain 120 or within the cerebral cortex of brain 120 via a burr hole in cranium 122 of patient 112, and electrically coupled to IMD 106 via one or more leads 114.

In the example shown in FIG. 1, electrodes 116, 118 of leads 114 are shown as ring electrodes. Ring electrodes may be used in AEF therapy applications because they are relatively simple to program and are capable of delivering an electrical field to any tissue adjacent to electrodes 116, 118. In other examples, electrodes 116, 118 may have different configurations. For example, in some examples, at least some of the electrodes 116, 118 of leads 114 may have a complex electrode array geometry that is capable of producing electrical fields of various shapes and electrical fields directed to different directions with respect to the lead. The complex electrode array geometry may include multiple electrodes (e.g., partial ring or segmented electrodes) around the outer perimeter of each lead 114, rather than one ring electrode, such as shown in FIGS. 5A and 5B. In this manner, electrical stimulation may be directed in a specific direction, such as alternating directions for alternating electrical fields, from leads 114 to provide AEF therapy. In some examples, one or more leads 114 may include insulation on a portion of the lead that may enable electrical field directionality such that the electrical current is directed to certain circumferential locations other than the insulated portion. In some examples, fewer electrodes may be used to generate smaller electrical fields specifically selected to affect target tissue during the AEF delivery while avoiding subjecting other tissues to the electric fields. In some examples, a housing of IMD 106 may include one or more stimulation and/or sensing electrodes. In alternative examples, leads 114 may have shapes other than elongated cylinders as shown in FIG. 1. For example, leads 114 may be paddle leads, spherical leads, bendable leads, or any other type of shape effective in treating patient 112 and/or minimizing invasiveness of leads 114.

In this manner, any electrode arrays may be designed to be placed surgically in a tumor void or bed and deliver electric fields to cover the interior volume of the debulked void therein. These electrode arrays may include conformable grids, volumetric “balloons,” multiple small electrodes placed individually within the void, surface anchorable electrodes applied with sutures or glue to the inside surface of the void, very long linear arrays wrapped around the circumference of the void or spiraled within, spring formed structures (nitinol or other compliant material) to expand to fill the volume, and hybrid arrays with paddle and/or grid elements to cover void surface and penetration elements to extend field perpendicular to the tissue surface. In other examples, an electrode array may include conductive fluid electrodes (e.g., an “injectrode” or something similar) or electrodes designed and created via rapid manufacturing techniques to fit a patient's specific tumor void.

In the example shown in FIG. 1, IMD 106 includes a memory to store a plurality of therapy programs that each define a set of therapy parameter values. In some examples, IMD 106 may select a therapy program from the memory based on various parameters, such as sensed patient parameters and the identified patient behaviors. IMD 106 may generate electrical stimulation based on the selected therapy program to deliver effective AEF therapy that reduces or prevents cancerous cell division or other cellular functions.

External programmer 104 wirelessly communicates with IMD 106 as needed to provide or retrieve therapy information. Programmer 104 is an external computing device that the user, e.g., a clinician and/or patient 112, may use to communicate with IMD 106. For example, programmer 104 may be a clinician programmer that the clinician uses to communicate with IMD 106 and program one or more therapy programs for IMD 106. Alternatively, programmer 104 may be a patient programmer that allows patient 112 to select programs and/or view and modify therapy parameters. The clinician programmer may include more programming features than the patient programmer. In other words, more complex or sensitive tasks may only be allowed by the clinician programmer to prevent an untrained patient from making undesirable changes to IMD 106. IMD 106 may also transmit notifications to programmer 104 for delivery to a user in response to detecting one or more problems with stimulation and/or detection of one or more trigger events for patient 112. Programmer 104 may enter a new programming session for the user to select new stimulation parameters for subsequent therapy. External programmer 104 may display estimated locations of target tissue locations and/or suggested stimulation parameter values for delivering electrical stimulation that affects the target tissue location.

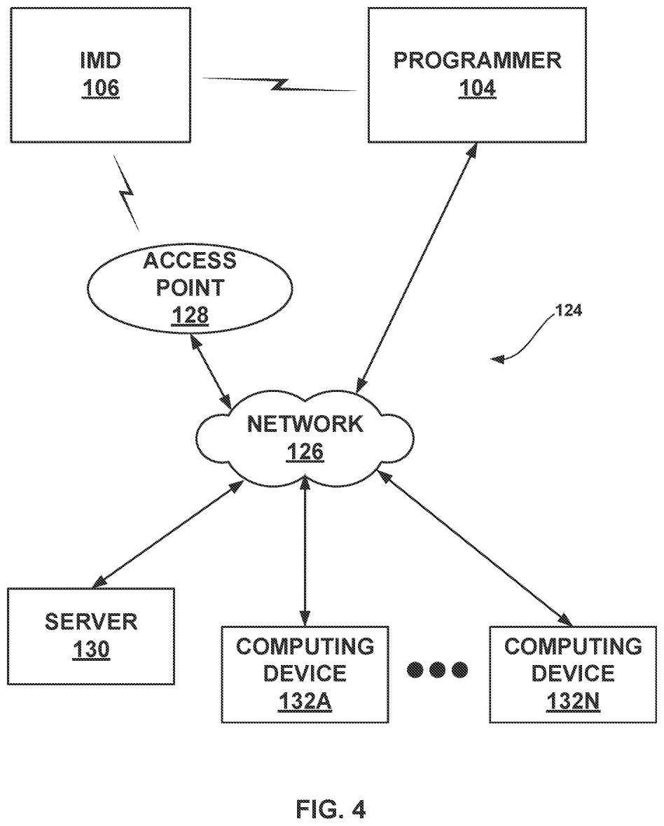

When programmer 104 is configured for use by the clinician, programmer 104 may be used to transmit initial programming information to IMD 106. This initial information may include hardware information, such as the type of leads 114 and the electrode arrangement, the position of leads 114 within brain 120, the configuration of electrode array 116, 118, initial programs defining therapy parameter values, and any other information the clinician desires to program into IMD 106. Programmer 104 may also be capable of completing functional tests (e.g., measuring the impedance of electrodes 116, 118 of leads 114 or the electric field strength at a strategic location on one of leads 114). In some examples, programmer 104 may receive sensed signals or representative information and perform the same techniques and functions attributed to IMD 106 herein. In other examples, a remote server (e.g., a standalone server or part of a cloud service as shown in FIG. 4) may perform the functions attributed to IMD 106, programmer 104, or any other devices described herein.

Programmer 104 may also be configured for use by patient 112. When configured as a patient programmer, programmer 104 may have limited functionality (compared to a clinician programmer) in order to prevent patient 112 from altering critical functions of IMD 106 or applications that may be detrimental to patient 112. In this manner, programmer 104 may only allow patient 112 to adjust values for certain therapy parameters or set an available range of values for a particular therapy parameter. In one example, a patient programmer may only allow for functions such as turning AEF therapy on or off and/or decreasing stimulation intensity. In some examples, programmer 104 may present an indication of delivery time for the patient, such as a screen that indicates the amount of time that the therapy is delivered and/or time the therapy has been off for each day, week, month, etc. For example, programmer 104 may present that “AEF therapy has been delivered for 85% of the time during the last week” or “AEF therapy has been delivered during 6 of the last 7 days.” In addition, programmer 104 may present remaining therapy time available before recharge is required when IMD 106 operates using a rechargeable power source.

Programmer 104 may also provide an indication to patient 112 when therapy is being delivered, when patient input has triggered a change in therapy or when the power source within programmer 104 or IMD 106 needs to be replaced or recharged. For example, programmer 112 may include an alert LED, may flash a message to patient 112 via a programmer display, generate an audible sound or somatosensory cue to confirm patient input was received, e.g., to indicate a patient state or to manually modify a therapy parameter.

Therapy system 100 may be implemented to provide chronic stimulation therapy to patient 112 over the course of several months or years. However, system 100 may also be employed on a trial basis to evaluate therapy before committing to full implantation. If implemented temporarily, some components of system 100 may not be implanted within patient 112. For example, patient 112 may be fitted with an external medical device, such as a trial stimulator, rather than IMD 106. The external medical device may be coupled to percutaneous leads or to implanted leads via a percutaneous extension. If the trial stimulator indicates AEF system 100 provides effective treatment to patient 112, the clinician may implant a chronic stimulator within patient 112 for relatively long-term treatment with AEF therapy.

Although IMD 106 is described as delivering electrical stimulation therapy to brain 120, IMD 106 may be configured to direct electrical stimulation to other anatomical regions of patient 112 in other examples. In other examples, system 100 may include an implantable drug pump in addition to, or in place of, IMD 106. Further, an IMD may provide other electrical stimulation such as spinal cord stimulation to treat other types of cancer or other diseases or disorders. In some embodiments, the therapy delivered by IMD 106 is designed to enhance the ability of particular drugs to pass through the blood-brain barrier, or is designed to enable particular drugs to pass through the blood-brain barrier. In other embodiments, the therapy delivered by IMD 106 is designed to enhance and/or enable cell membrane permeabilization for the purpose of mediating cell transfection or enhancing viral delivery to target cells. By being designed to achieve these goals, ID 106 may be configured (via specific stimulation parameter values) to deliver electrical field therapy that increases blood-brain barrier permeabilization and/or enhances cell membrane permeabilization.

According to the techniques of the disclosure, system 100 may include processing circuitry configured to receive a request to deliver alternating electric field (AEF) therapy, determine therapy parameter values that define the AEF therapy, wherein the AEF therapy comprises delivery of a first electric field and a second electric field, control IMD 106 to deliver the first electric field from a first electrode combination of implanted electrodes, and control IMD 106 to deliver, alternating with the first electric field, the second electric field from a second electrode combination of implanted electrodes different than the first electrode combination. The request may be via user input and/or an automated system request to start AEF therapy delivery.

The electrical fields that IMD 106 alternates over time to produce the AEF therapy may involve different electrode combinations and/or different methods for alternating the electrical fields between different electrode combinations (e.g., different electrodes and/or different polarities of the same or different electrodes). In one example, the first electrode combination includes a first set of electrodes defined as cathodes and a second set of electrodes defined as anodes, and the second electrode combination includes the first set of electrodes defined as anodes and the second set of electrodes defined as cathodes.

In one example in which ID 106 utilizes 4 different implantable leads, the first electrode combination includes a first set of anodes carried by a first lead, the second electrode combination includes a first set of cathodes carried by a second lead different than the first lead, the third electrode combination includes a second set of anodes carried by a third lead different than the first lead and the second lead, and the fourth electrode combination includes a second set of cathodes carried by a fourth lead different than the first lead, the second lead, and the third lead. The first and second electrical fields may generally be orthogonal or oblique to each other. In another example in which two leads are used to deliver AEF therapy, the first electrode combination includes a first set of anodes carried by a first lead, the second electrode combination includes a first set of cathodes carried by a second lead different than the first lead, the third electrode combination comprises a second set of anodes carried by the second lead, and the fourth electrode combination comprises a second set of cathodes carried by the first lead. In some examples, the AEF therapy may include alternating or switching between the first electrode combination and the second electrode combination, where some or all of electrodes of the first lead switch between operating as anodes in the first electrode combination and operating as cathodes in the second electrode combination, and electrodes of the second lead switch between operating as cathodes in the first electrode combination and operating as anodes in the second electrode combination. In other examples, the first and second electrode combinations may utilize completely different electrodes for each anodes and cathodes. In other examples, each electrode combination may utilize one lead for anodes and a different lead for cathodes. The different electrode combinations used to alternate electric fields may share leads or utilize separate leads for each electrode combination. These are only some of the different methods for generating alternating electric fields from an array of implanted electrodes, as other examples are also contemplated. For example, IMD 106 may instead alternate, or sweep through, three or more different electrical fields generated from respective electrode combinations. These larger number of electrical fields may effectively treat a larger number of cells depending on the location of the cells within respect to the location of the implanted electrodes.

Although alternating electric field therapy is generally described as delivering two different electric fields, three or more electric fields may be delivered in other examples. For example, IMD 106 may be configured to deliver three electric fields that are all orthogonal to each other. In other examples, four or more different electric fields may be delivered to the cells in order to affect cells oriented in a variety of different directions. In this manner, IMD 106 may deliver tens or hundreds of different electric fields having different vectors (limited only by the available electrode combinations for delivering the electric fields) by sweeping through a sequence of these electric fields or otherwise delivering these different electric fields in order to affect cells having different orientations. Three electric fields with all different directional vectors may enable three dimensional electrical field treatment of the target tissue.

In some examples, IMD 106 is configured to cycle the AEF therapy on and off according to a predetermined schedule. This predetermined cycle may be set according to the speed of tumor cell division in order to cycle the AEF therapy at a rate that enables the tumor cells are guaranteed to experience a relevant field at least once per cell divisional time to inhibit the division of the cells. In other examples, IMD 106 may be configured to receive temperature data indicative of a temperature of tissue that receives the AEF therapy, determine that the temperature exceeds a threshold temperature, responsive to determining that the temperature exceeds the threshold temperature, terminate delivery of the AEF therapy. This temperature monitoring may reduce the risk of tissue damage due to electrical field induced tissue heating.

IMD 106 may generally use the same pulse or signal frequency for generating the first and second electrical fields of the AEF therapy. In one example, the frequency may be approximately 150 kHz. In another example, the frequency may be approximately 200 kHz. In general, the frequency may be selected from a range of approximately 100 kHz through 300 kHz, but frequencies higher or lower than this range may be used in other examples. In some examples, the frequencies employed by ID 106 are selected based on the types of cells targeted for treatment. For example, if targeting cancer cells of a certain size (e.g., 13 micrometers in diameter), the IMD 106 delivers therapy with a frequency (e.g., 200 kHz) at which therapy will be more effective for that cell size. In some examples, the frequency or range of frequencies at which the electrical fields are delivered may be selected based on a workup of a patient biopsy or based on a lookup table according to the tumor type and associated distributions of cell sizes. In some examples, the minimum or maximum frequencies may be selected in order to avoid affecting sizes of healthy cells within the electric fields that may differ from the size of the tumor cells.

In some examples, IMD 106 may switch between two or more different frequencies during delivery of the AEF therapy. In one example, IMD 106 may be configured to adjust a frequency of the first electric field and the second electric field according to a predetermined schedule. In this manner, IMD 106 can employ a variety of frequencies to target a variety of cell sizes during a treatment cycle. In one example, IMD 106 can sweep through different frequencies in order to treat cells of various sizes that may react to different frequencies, as described further herein. In other examples, IMD 106 may be configured to receive an indication that a trigger event occurred, and responsive to receiving the indication that the trigger event occurred, adjust a frequency of the first electric field and the second electric field. The trigger event may be patient activity (e.g., the patient is moving or the patient is sleeping—to target circadian timing of the patient and possibly associated cell growth cycles), detected brain activity, or any other type of trigger event that may indicate AEF therapy should or should not be delivered. Other trigger events may include therapeutic events such as delivery of another therapy (e.g., chemotherapy or other moderation therapy) in order to synchronize the effectiveness of each therapy or the totality of the therapies. In some examples, the first electric field is defined by a first frequency, and the second electric field is defined by a second frequency different than the first frequency.

In some examples, system 100 may be configured to determine, or recommend for user approval, one or more stimulation parameters that at least partially define the AEF therapy. For example, programmer 104 may include a user interface configured to receive user input indicative of target tissue to receive AEF therapy. Programmer 104 may be configured to determine, based on the user input, the first electrode combination and the second electrode combination. In this manner, system 100 can achieve therapy of desired tissue, such as a glioblastoma tumor or other tissue of concern. Alternatively, or in addition, programmer 104 may include a user interface configured to receive user input indicative of tissue to avoid receiving AEF therapy. Since programmer 104 may be a patient or clinician programmer, the user interface may be configured to receive input from a clinician or a patient. However, in some examples, the user interface may provide additional options or expanded customizability for clinicians when compared to patients. In some embodiments, the IMD 106 is configured to determine, e.g., using signals sensed by the electrodes, that electric fields are reaching a particular tissue structure. In some examples, one or more of the electrodes may be located in or near a non-target tissue to indicate the presence of electric fields at the non-target tissue (e.g., a specific recording electrode(s)). The IMD 106 (either alone or in combination with external devices) can adjust the applied therapy to reduce or eliminate the applied electric fields (or the effects of the applied electric fields) at that particular tissue structure. Programmer 104 may then determine, based on the user input, the first electrode combination and the second electrode combination. System 100 can then attempt to reduce the effect of AEF therapy on non-target tissues. In some examples, system 100 may receive user input indicative of target tissue and/or tissue to avoid from a remote device over a network to support remote programmer options for system 100.

System 100 may also determine stimulation parameters based on feedback regarding the state of patient 112 and/or tissue of the patient. For example, programmer 104 and/or IMD 106 may adjust one or more stimulation parameters that at least partially defines the AEF therapy based on histological data obtained from a sample of tissue affected by the AEF therapy. In another example, programmer 104 and/or IMD 106 may determine target tissue for AEF therapy based on water content data obtained from magnetic resonance imaging (MRI) data, and determine, based on the target tissue, the first electrode combination and the second electrode combination for delivery of the AEF therapy. In some examples, determining the electrode combinations may include determining the location, e.g., based on predictive computational models of electric field intensity in tissue, at which one or more leads should be located in order to deliver AEF therapy to the target tissue. As another example, programmer 104 and/or IMD 106 may determine target tissue for AEF therapy based on impedance tomography data obtained from sensed electrical potentials sensed from two or more of the implanted electrodes (and/or external electrodes disposed to record electric fields), and determine, based on the target tissue, at least the first electrode combination and the second electrode combination to deliver the AEF therapy. System 100 may also map AEF features to anatomy to inform AEF therapy planning and/or adjustments over time. For example, programmer 104 may be configured to generate an AEF dosimetry metric for anatomy that receives the AEF therapy and map the AEF dosimetry across target tissue of the anatomy. This AEF dosimetry map may inform which tissues within the anatomy are receiving different strengths of the electrical fields. Programmer 104 may also display the map of the AEF dosimetry with respect to the anatomy.

IMD 106 may alternate the electrical fields in AEF therapy by delivering the electrical fields from different electrodes and/or electrodes with different polarities. In one example, IMD 106 may continually shift the polarities of the electrodes in one direction with respect to the electrode array. The first electrode combination may include a first set of electrodes defined as cathodes and a second set of electrodes defined as anodes, the second electrode combination may include a third set of electrodes defined as anodes and a fourth set of electrodes defined as cathodes, where the third set of electrodes are adjacent to the first set of electrodes in one direction on a first lead, and the fourth set of electrodes are adjacent to the second set of electrodes in the one direction on a second lead. In some examples, electrode combinations adjacent each other may be 180 degrees out of phase with each other in order to provide a maximum amount of change in voltage between the tissue separating the adjacent electrode contacts. In another example, the electrode combinations may be selected from a cube configuration where the selectable electrodes for each electrode combination form the eight vertices of a cube. In this example, the first electrode combination includes a first set of electrodes defined as cathodes and a second set of electrodes defined as anodes in a first paired configuration from the cube configuration, and the second electrode combination includes the first set of electrodes defined as anodes and the second set of electrodes defined as cathodes in a second paired configuration from the cube configuration.

As shown in FIG. 1, the electrodes (e.g., at least two electrodes) used to deliver the AEF therapy are carried by an electrode array positioned adjacent a resection bed of tissue. In some examples, at least two electrodes of the implanted electrodes used to deliver AEF therapy are subcutaneous electrodes (e.g., electrodes implanted beneath the skin and superficial of the bone). In some examples, one or more electrodes may be implanted on the inside of the removed skull portion during surgery in order to provide a relatively easy implantation of electrodes within the skull but outside of the brain. In other examples, a system may deliver the AEF therapy using a plurality of external cutaneous electrodes in combination with a plurality of implanted electrodes. For example, a first electrical field of the AEF therapy may be delivered between two or more external electrodes and the second electrical field of the AEF therapy is delivered between two or more implanted electrodes. In other example, two or more electrical fields that create the AEF therapy each utilize one or more external electrodes. In some examples, the current passes between only external electrodes or between only implanted electrodes. In other examples, one or more electrical fields may be created between any combination of external and implanted electrodes. An external device may deliver current to the external electrodes, either independent controlled or controlled based on communication between the external device and IMD 106.

Generally, AEF therapy is described herein as a treatment to already present tumors, such as glioblastomas. In other examples, the application of AEF therapy can reduce the extent of metastatic tumor burden and seeding of tumors from a remote tumor source. Therefore, AEF treatment could be delivered to protect tissue regions from metastatic spread. For example, AEF could be utilized to provide global brain protection in the setting of a known malignant tumor within the body, particularly those that have a propensity for cerebral dissemination (e.g., Melanoma). AEF could be delivered to prevent additional metastatic spread of tumor within the organ system of current metastatic dissemination. In addition, AEF implant planning could be provided for the protection of certain neurological function (e.g., motor function), such that the implant system 100 would be focused on treatment to the pre-central gyrus and/or corticospinal tract to preserve its function and avoid seeding.

An AEF delivery implant (e.g., IMD 106 and leads 114) could be utilized to prophylactically treat a body region that is expected to have a high risk for metastatic dissemination (e.g., a presumed location where tumor would progress next). An example of this is the axillary lymph nodes in the setting of a newly diagnosed breast cancer. Lymphatic channels have predictable flow and are common highways for metastatic dissemination. Therefore, implantation strategies that focus on the systematic treatment of these highways could meaningfully impact the propensity and capability for tumors to metastatically spread.

AEF delivery could also utilize electrodes within arteries to permit wider control of metastatic spread. Put another way, AEF delivery could reduce the likelihood of metastatic tumor cells to exit the blood stream and seed other regions within the body. In one example, treatment of the carotid arteries could reduce the ability for tumor cells to exit the bloodstream and invade the cerebral tissue. AEF delivery within the arteries could provide advantageous pharmacokinetic impacts on drugs within the tumor region and therefore justify intra-arterial placement of electrodes for AEF delivery. In this manner, AEF therapy described herein may be applied to numerous different tissues and for numerous different reasons. For example, AEF therapy or other modulated electric field therapy may be provided as a preventative therapy to tissue not yet diagnosed as cancerous but at risk for tumor occurrence based on one or more characteristics such as genetic markers, environmental factors, or any other risk factors.

The architecture of system 100 illustrated in FIG. 1 is shown as an example. The techniques as set forth in this disclosure may be implemented in the example system 100 of FIG. 1, as well as other types of systems not described specifically herein. Nothing in this disclosure should be construed so as to limit the techniques of this disclosure to the example architecture illustrated by FIG. 1.

System 100 is generally described as including IMD 106 and external programmer 104. However, in other examples, an external medical device may be configured to perform any of the techniques described herein or described with respect to IMD 106. The external medical device may be coupled to percutaneous leads or other devices that pass through the skin in order to dispose implanted electrodes at various locations within patient for at least partially delivering electric field therapy and/or sensing signals as described herein. Additionally, or alternatively, the external device may be coupled to external electrodes configured to at least partially deliver electric field therapy and/or sense signals as described herein. The external medical device may be configured to communicate with programmer 104 and/or partially or fully incorporate structures to perform the various functionality described with respect to programmer 104.

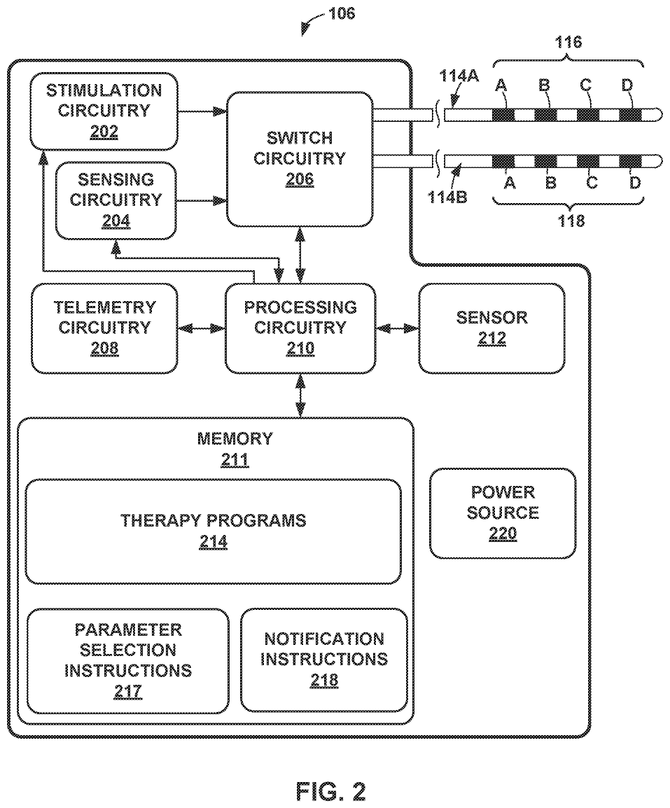

FIG. 2 is a block diagram of the example IMD 106 of FIG. 1 for delivering AEF therapy. In the example shown in FIG. 2, IMD 106 includes processing circuitry 210, memory 211, stimulation generator 202, sensing module 204, switch module 206, telemetry module 208, sensor 212, and power source 220. Each of these modules may be or include electrical circuitry configured to perform the functions attributed to each respective module. For example, processing circuitry 210 may include processing circuitry, switch module 206 may include switch circuitry, sensing module 204 may include sensing circuitry, and telemetry module 208 may include telemetry circuitry. Switch module 206 may not be used for multiple current source and sink configurations, but one or more switches may still be used to disconnect sensing module 204 from the source and sinks in such a configuration. Memory 211 may include any volatile or non-volatile media, such as a random-access memory (RAM), read only memory (ROM), non-volatile RAM (NVRAM), electrically erasable programmable ROM (EEPROM), flash memory, and the like. Memory 211 may store computer-readable instructions that, when executed by processing circuitry 210, cause IMD 106 to perform various functions. Memory 211 may be a storage device or other non-transitory medium.

In the example shown in FIG. 2, memory 211 stores therapy programs 214 that include respective stimulation parameter sets that define AEF therapy. Each stored therapy program 214 defines a particular set of electrical stimulation parameters (e.g., a therapy parameter set), such as a stimulation electrode combination, electrode polarity, current or voltage amplitude, pulse width, and pulse rate. In some examples, individual therapy programs may be stored as a therapy group, which defines a set of therapy programs with which stimulation may be generated.

Memory 211 may also include parameter selection instructions 217 and notification instructions 218. Parameter selection instructions 217 may include instructions that control processing circuitry 210 selecting different stimulation parameter values such as electrode combinations, amplitudes, pulse frequencies, or other parameter values for compensating for various locations of target tissue or feedback related to changes in patient condition or tissue state. Parameter selection instructions 217 may include instructions for processing circuitry 210 to select parameter values based on various feedback variables. Notification instructions 218 may define instructions that control processing circuitry 210 actions such as transmitting an alert or other notification to an external device, such as programmer 104, that therapy is on or off, or if changes to AEF therapy have been made or are recommended.

In some examples, the sense and stimulation electrode combinations may include the same subset of electrodes 116, 118, a housing of IMD 106 functioning as an electrode, or may include different subsets or combinations of such electrodes. Thus, memory 211 can store a plurality of sense electrode combinations and, for each sense electrode combination, store information identifying the stimulation electrode combination that is associated with the respective sense electrode combination. The associations between sense and stimulation electrode combinations can be determined, e.g., by a clinician or automatically by processing circuitry 210. In some examples, corresponding sense and stimulation electrode combinations may comprise some or all of the same electrodes. In other examples, however, some or all of the electrodes in corresponding sense and stimulation electrode combinations may be different. For example, a stimulation electrode combination may include more electrodes than the corresponding sense electrode combination in order to increase the efficacy of the AEF therapy.

Stimulation generator 202, under the control of processing circuitry 210, generates stimulation signals for delivery to patient 112 via selected combinations of electrodes 116, 118. An example range of electrical stimulation parameters believed to be effective in AEF therapy to manage cellular activity include:
1. Frequency (e.g., waveform frequency or pulse rate): between approximately 50 kHz and approximately 500 kHz, such as between approximately 100 kHz to 300 kHz, or such as approximately 150 kHz or 200 kHz.
2. In the case of a voltage controlled system, Voltage Amplitude: between approximately 0.1 volts and approximately 50 volts, such as between approximately 2 volts and approximately 10 volts.
3. In the alternative case of a current controlled system, Current Amplitude: between approximately 0.2 milliamps to approximately 100 milliamps, such as between approximately 1.3 milliamps and approximately 2.0 milliamps.
4. Pulse Width: between approximately 1 microseconds and approximately 10 microseconds, such as between approximately 1 microseconds and approximately 5 microseconds, or between approximately 2 microseconds and approximately 10 microseconds.
5. Cycle time (e.g., communication time), which is the time a waveform remains consistent before switching off or switching to a new waveform. The cycle time may be selected from a range of 30 seconds and 30 minutes, or within a range from 1 minute to 10 minutes. Shorter and longer cycle times may be used in other examples.

Accordingly, in some examples, stimulation generator 202 generates electrical stimulation signals in accordance with the electrical stimulation parameters noted above. Other ranges of therapy parameter values may also be useful, and may depend on the target stimulation site within patient 112. While stimulation pulses are described, stimulation signals may be of any form, such as continuous-time signals (e.g., sine waves) or the like. Stimulation signals configured to elicit ECAPs or other evoked physiological signals may be similar or different from the above parameter value ranges. In addition, sensing circuitry 204 may be configured to sense signals via one or more electrode combinations on one or more leads 114 (e.g., the same or different electrodes may deliver stimulation and sense electrical signals).

Processing circuitry 210 may include fixed function processing circuitry and/or programmable processing circuitry, and may comprise, for example, any one or more of a microprocessor, a controller, a digital signal processor (DSP), an application specific integrated circuit (ASIC), a field-programmable gate array (FPGA), discrete logic circuitry, or any other processing circuitry configured to provide the functions attributed to processing circuitry 210 herein may be embodied as firmware, hardware, software or any combination thereof. Processing circuitry 210 may control stimulation generator 202 according to therapy programs 214 stored in memory 211 to apply particular stimulation parameter values specified by one or more of programs, such as voltage amplitude or current amplitude, pulse width, or pulse rate.

In the example shown in FIG. 2, the set of electrodes 116 includes electrodes 116A, 116B, 116C, and 116D, and the set of electrodes 118 includes electrodes 118A, 118B, 118C, and 118D. Processing circuitry 210 also controls switch module 206 to apply the stimulation signals generated by stimulation generator 202 to selected combinations of electrodes 116, 118. In particular, switch module 204 may couple stimulation signals to selected conductors within leads 114, which, in turn, deliver the stimulation signals across selected electrodes 116, 118. Switch module 206 may be a switch array, switch matrix, multiplexer, or any other type of switching module configured to selectively couple stimulation energy to selected electrodes 116, 118 and to selectively sense neurological brain signals with selected electrodes 116, 118. Hence, stimulation generator 202 is coupled to electrodes 116, 118 via switch module 206 and conductors within leads 114. In some examples, however, IMD 106 does not include switch module 206, such as if each electrode is assigned a respective current and sink (e.g., independent current source).

Stimulation generator 202 may be a single channel or multi-channel stimulation generator. In particular, stimulation generator 202 may be capable of delivering a single stimulation pulse, multiple stimulation pulses, or a continuous signal at a given time via a single electrode combination or multiple stimulation pulses at a given time via multiple electrode combinations. In some examples, however, stimulation generator 202 and switch module 206 may be configured to deliver multiple channels on a time-interleaved basis. For example, switch module 206 may serve to time divide the output of stimulation generator 202 across different electrode combinations at different times to deliver multiple programs or channels of stimulation energy to patient 112 (e.g., cycling between regimes of stimulation on a fixed or variable sequence). Alternatively, stimulation generator 202 may comprise multiple voltage or current sources and sinks that are coupled to respective electrodes to drive the electrodes as cathodes or anodes. In this example, IMD 106 may not require the functionality of switch module 206 for time-interleaved multiplexing of stimulation via different electrodes.

Electrodes 116, 118 on respective leads 114 may be constructed of a variety of different designs. For example, one or both of leads 114 may include two or more electrodes at each longitudinal location along the length of the lead, such as multiple electrodes at different perimeter locations around the perimeter of the lead at each of the locations A, B, C, and D. On one example, the electrodes may be electrically coupled to switch module 206 via respective wires that are straight or coiled within the housing or the lead and run to a connector at the proximal end of the lead. In another example, each of the electrodes of the lead may be electrodes deposited on a thin film. The thin film may include an electrically conductive trace for each electrode that runs the length of the thin film to a proximal end connector. The thin film may then be wrapped (e.g., a helical wrap) around an internal member to form the lead 114. These and other constructions may be used to create a lead with a complex electrode geometry.

Although sensing module 204 is incorporated into a common housing with stimulation generator 202 and processing circuitry 210 in FIG. 2, in other examples, sensing module 204 may be in a separate housing from IMD 106 and may communicate with processing circuitry 210 via wired or wireless communication techniques. Example neurological brain signals include, but are not limited to, a signal generated from local field potentials (LFPs) within one or more regions of brain 28. EEG and ECoG signals are examples of other types of electrical signals that may be measured within brain 120. Other examples include sensed signals representative of electric field or voltage gradients caused by a remote electrode as recorded by a proximal electrode or electrode pair. Instead of, or in addition to, LFPs, IMD 106 may be configured to detect patterns of single-unit activity and/or multi-unit activity. IMD 106 may sample this activity at rates above 1,000 Hz, and in some examples within a frequency range of 6,000 Hz to 500,000 Hz. IMD 106 may identify the wave-shape of single units and/or an envelope of unit modulation that may be features used to differentiate or rank electrodes. In some examples, this technique may include phase-amplitude coupling to the envelope or to specific frequency bands in the LFPs sensed from the same or different electrodes. In some examples, the sampling technique may be set to identify the electric field strength at any location. For example, IMD 106 may include a peak following circuitry that holds the amplitude of a field of a specific frequency for later sampling. Alternatively, the response of a resonant circuit may be tuned to the AEF frequency might sampled to infer the field strength of the desired signal.

Sensor 212 may include one or more sensing elements that sense values of a respective patient parameter, such as patient activity (e.g., movement and/or sleep). For example, sensor 212 may include one or more accelerometers, optical sensors, chemical sensors, temperature sensors, pressure sensors, or any other types of sensors. Sensor 212 may output patient parameter values that may be used as feedback to control delivery of AEF therapy. IMD 106 may include additional sensors within the housing of IMD 106 and/or coupled via one of leads 114 or other leads. In addition, IMD 106 may receive sensor signals wirelessly from remote sensors via telemetry module 208, for example. In some examples, one or more of these remote sensors may be external to patient (e.g., carried on the external surface of the skin, attached to clothing, or otherwise positioned external to the patient).

Telemetry module 208 supports wireless communication between IMD 106 and an external programmer 104 or another computing device under the control of processing circuitry 210. Processing circuitry 210 of IMD 106 may receive, as updates to programs, values for various stimulation parameters such as magnitude and electrode combination, from programmer 104 via telemetry module 208. The updates to the therapy programs may be stored within therapy programs 214 portion of memory 211. In addition, processing circuitry 210 may control telemetry module 208 to transmit alerts or other information to programmer 104 that indicate a lead moved with respect to tissue. Telemetry module 208 in IMD 106, as well as telemetry modules in other devices and systems described herein, such as programmer 104, may accomplish communication by radiofrequency (RF) communication techniques. In addition, telemetry module 208 may communicate with external medical device programmer 104 via proximal inductive interaction of IMD 106 with programmer 104. Accordingly, telemetry module 208 may send information to external programmer 104 on a continuous basis, at periodic intervals, or upon request from IMD 106 or programmer 104.

Power source 220 delivers operating power to various components of IMD 106. Power source 220 may include a small rechargeable or non-rechargeable battery and a power generation circuit to produce the operating power. Recharging may be accomplished through proximal inductive interaction between an external charger and an inductive charging coil within IMD 220. In some examples, power requirements may be small enough to allow IMD 220 to utilize patient motion and implement a kinetic energy-scavenging device to trickle charge a rechargeable battery. In other examples, traditional batteries may be used for a limited period of time. In other examples, IMD 106 may include a power receiving antenna an corresponding circuitry to continually receive external power that enables IMD 106 to deliver electric field therapy indefinitely without possible internal power source drain.

According to the techniques of the disclosure, processing circuitry 210 of IMD 106 delivers, electrodes 116, 118 interposed along leads 114 (and optionally switch module 206), electrical stimulation therapy to patient 112. The AEF therapy is defined by one or more therapy programs 214 having one or more parameters stored within memory 211. For example, the one or more parameters include a current amplitude (for a current-controlled system) or a voltage amplitude (for a voltage-controlled system), a pulse rate or frequency, and a pulse width, or quantity of pulses per cycle. In examples where the electrical stimulation is delivered according to a “burst” of pulses, or a series of electrical pulses defined by an “on-time” and an “off-time,” the one or more parameters may further define one or more of a number of pulses per burst, an on-time, and an off-time.

In some examples, the plurality of electrode combinations includes at least one electrode combination comprising electrodes disposed at different positions around a perimeter of the lead. In some examples, at least one electrode combination includes electrodes disposed at different positions along a longitudinal axis of the lead implanted in the patient. In this manner, the plurality of electrode combinations may include electrode combinations where each electrode combination has electrodes at different positions around the perimeter of the lead, electrode combinations where each electrode combination has electrode at different positions along the longitudinal axis of the lead, or electrode combinations that include both electrode combinations with electrodes at different positions around the perimeter of the lead and different positions along the longitudinal axis of the lead.

The one or more features determined from the sensed electrical signals may include or represent different characteristics of the sensed electrical signals. Example features of the sensed electrical signals may include voltage (peak or average), orientation of resulting electric field, impedance, spectral power, one or more frequencies, one or more frequency bands, or any other characteristics of the sensed signals. In this manner, the one or more features may be features in the time domain, frequency domain, or any other signal domain relevant to identify the sensed signals. For example, processing circuitry 210 may be configured to determine the one or more features by at least determining a sensed voltage for each electrode combination of the plurality of electrode combinations. In some examples, processing circuitry 210 may be configured to determine the one or more features by at least determining at least one of a power, a frequency band, a time domain feature, and/or a frequency domain feature of each signal of the plurality of signals from respective electrode combinations of the plurality of electrode combinations.

Processing circuitry 210 may be configured to control a user interface to display various elements and aspects related to the planning and delivery of AEF therapy. For example, processing circuitry 210 may transmit sensed data related to AEF therapy such as impedance tomography, stimulation parameters, patent activity, and any other data to programmer 104 for display to the user via the user interface. In some examples, processing circuitry 210 may be configured to receive user selection of a value for one or more stimulation parameters that at least partially defines AEF therapy. For example, the user interface may present a visual indication of the estimated location of the target tissue and electrical field strength variations over different locations of the target tissue.

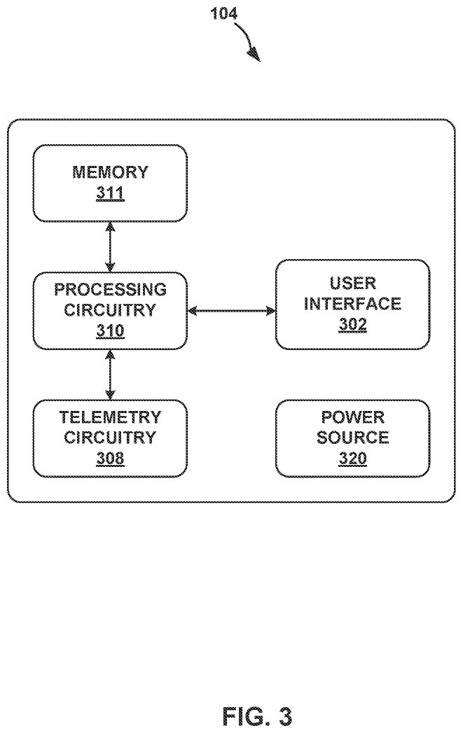

FIG. 3 is a block diagram of the external programmer 104 of FIG. 1 for planning and/or controlling delivery of AEF therapy according to an example of the techniques of the disclosure. Although programmer 104 may generally be described as a hand-held device, programmer 104 may be a larger portable device or a more stationary device. In some examples, programmer 104 may be referred to as a tablet computing device or a smart phone computing device. In addition, in other examples, programmer 104 may be included as part of a bed-side monitor, an external charging device or include the functionality of an external charging device. As illustrated in FIG. 3, programmer 104 may include a processing circuitry 310, memory 311, user interface 302, telemetry module 308, and power source 320. Memory 311 may store instructions that, when executed by processing circuitry 310, cause processing circuitry 310 and external programmer 104 to provide the functionality ascribed to external programmer 104 throughout this disclosure. Each of these components, or modules, may include electrical circuitry that is configured to perform some or all of the functionality described herein. For example, processing circuitry 310 may include processing circuitry configured to perform the processes discussed with respect to processing circuitry 310.

In general, programmer 104 comprises any suitable arrangement of hardware, alone or in combination with software and/or firmware, to perform the techniques attributed to programmer 104, and processing circuitry 310, user interface 302, and telemetry module 308 of programmer 104. In various examples, programmer 104 may include one or more processors, which may include fixed function processing circuitry and/or programmable processing circuitry, as formed by, for example, one or more microprocessors, DSPs, ASICs, FPGAs, or any other equivalent integrated or discrete logic circuitry, as well as any combinations of such components. Programmer 104 also, in various examples, may include a memory 311, such as RAM, ROM, PROM, EPROM, EEPROM, flash memory, a hard disk, a CD-ROM, comprising executable instructions for causing the one or more processors to perform the actions attributed to them. Moreover, although processing circuitry 310 and telemetry module 308 are described as separate modules, in some examples, processing circuitry 310 and telemetry module 308 may be functionally integrated with one another. In some examples, processing circuitry 310 and telemetry module 308 correspond to individual hardware units, such as ASICs, DSPs, FPGAs, or other hardware units.

Memory 311 (e.g., a storage device) may store instructions that, when executed by processing circuitry 310, cause processing circuitry 310 and programmer 104 to provide the functionality ascribed to programmer 104 throughout this disclosure. For example, memory 311 may include instructions that cause processing circuitry 310 to obtain a parameter set from memory, present a model of patient anatomy for predicting electrical field strengths, provide an interface that recommends or otherwise facilitates parameter value selection, or receive a user input and send a corresponding command to IMD 106, or instructions for any other functionality. In addition, memory 311 may include a plurality of programs, where each program includes a parameter set that defines stimulation therapy.

User interface 302 may include a button or keypad, lights, a speaker for voice commands, a display, such as a liquid crystal (LCD), light-emitting diode (LED), or organic light-emitting diode (OLED). In some examples the display may be a presence-sensitive screen, such as a touch screen. User interface 302 may be configured to display any information related to the delivery of stimulation therapy, detected trigger events, progression of therapy, suggested stimulation parameter values, sensed patient parameter values, or any other such information. User interface 302 may also receive user input via user interface 302. The input may be, for example, in the form of pressing a button on a keypad or selecting an icon from a touch screen.

Telemetry module 308 may support wireless communication between IMD 106 and programmer 104 under the control of processing circuitry 310. Telemetry module 308 may also be configured to communicate with another computing device via wireless communication techniques, or direct communication through a wired connection. In some examples, telemetry module 308 provides wireless communication via an RF or proximal inductive medium. In some examples, telemetry module 308 includes an antenna, which may take on a variety of forms, such as an internal or external antenna. In some examples, IMD 106 and/or programmer 104 may communicate with remote servers via one or more cloud-services in order to deliver and/or receive information between a clinic and/or programmer.

Examples of local wireless communication techniques that may be employed to facilitate communication between programmer 104 and IMD 106 include RF communication according to the 802.11 or Bluetooth specification sets or other standard or proprietary telemetry protocols. Security protocols and encryption techniques may be applied to enhance the security of the communication techniques. In addition, other external devices may be capable of communicating with programmer 104 without needing to establish a secure wireless connection. As described herein, telemetry module 308 may be configured to transmit a spatial electrode movement pattern or other stimulation parameter values to IMD 106 for delivery of stimulation therapy.

FIG. 4 is a block diagram illustrating an example system 124 that includes an external device, such as a server 130, and one or more computing devices 132A-132N, that are coupled to IMD 106 and external programmer 104 shown in FIG. 1 via a network 126. In this example, IMD 106 may use its telemetry circuit to communicate with external programmer 104 via a first wireless connection, and to communication with an access point 128 via a second wireless connection.

In the example of FIG. 4, access point 128, external programmer 104, server 130, and computing devices 132A-132N are interconnected, and able to communicate with each other, through network 126. In some cases, one or more of access point 128, external programmer 104, server 130, and computing devices 132A-132N may be coupled to network 126 through one or more wireless connections. IMD 106, external programmer 104, server 130, and computing devices 132A-132N may each comprise one or more processors, such as one or more microprocessors, digital signal processors (DSPs), application specific integrated circuits (ASICs), field programmable gate arrays (FPGAs), programmable logic circuitry, or the like, that may perform various functions and operations, such as those described in this disclosure.

Access point 128 may comprise a device, such as a home monitoring device, that connects to network 126 via any of a variety of connections, such as telephone dial-up, digital subscriber line (DSL), or cable modem connections. In other embodiments, access point 128 may be coupled to network 126 through different forms of connections, including wired or wireless connections.

During operation, IMD 106 may collect and store various forms of data. For example, IMD 106 may collect sensed posture state information during therapy that indicate how patient 112 moves throughout each day. IMD 106 may store usage statistics (e.g., delivery times in hours per day, percentage of on time, compliance to the dosing schedule, etc.) for later presentation to a user or otherwise evaluating therapy and/or patient compliance. In some cases, IMD 106 may directly analyze the collected data to evaluate the status of the patient and the delivery of AEF therapy or any other aspects of the patient. In other cases, however, IMD 106 may send stored data relating to AEF therapy to external programmer 104 and/or server 130, either wirelessly or via access point 128 and network 126, for remote processing and analysis.

For example, IMD 106 may sense, process, trend and evaluate sensed data and/or AEF therapy information. This communication may occur in real time, and network 126 may allow a remote clinician to review the data representative of AEF therapy by receiving a presentation of the data on a remote display, e.g., computing device 132A. Alternatively, processing, trending and evaluation functions may be distributed to other devices such as external programmer 104 or server 130, which are coupled to network 126. In addition, AEF therapy data may be archived by any of such devices, e.g., for later retrieval and analysis by a clinician.

In some cases, server 130 may be configured to provide a secure storage site for archival of AEF therapy information that has been collected from IMD 106 and/or external programmer 104. Network 126 may comprise a local area network, wide area network, or global network, such as the Internet. In other cases, external programmer 104 or server 130 may assemble AEF therapy information in web pages or other documents for viewing by trained professionals, such as clinicians, via viewing terminals associated with computing devices 132A-132N. System 124 may be implemented, in some aspects, with general network technology and functionality similar to that provided by the Medtronic CareLink® Network developed by Medtronic, Inc., of Minneapolis, MN.

Although some examples of the disclosure may involve AEF therapy information and data, system 124 may be employed to distribute any information relating to the treatment of patient 112 and the operation of any device associated therewith. For example, system 124 may allow therapy errors or device errors to be immediately reported to the clinician. In addition, system 124 may allow the clinician to remotely intervene in the therapy and reprogram IMD 106, patient programmer 104, or communicate with patient 112. In an additional example, the clinician may utilize system 124 to monitor multiple patients and share data with other clinicians in an effort to coordinate rapid evolution of effective treatment of patients.

FIGS. 5A and 5B are conceptual diagrams of example leads 400 and 410, respectively, with respective electrodes carried by the lead. As shown in FIGS. 5A and 5B, leads 400 and 410 are embodiments of leads 114 shown in FIG. 1. As shown in FIG. 5A, lead 400 includes four electrode levels 404 (includes levels 404A-404D) mounted at various lengths of lead housing 402. Lead 400 is inserted into through cranium 122 to a target position within brain 18.

Lead 400 is implanted within brain 120 at a location determined by the clinician that may be near an anatomical region to receive AEF therapy, such as a tumor location or resection bed. Electrode levels 404A, 404B, 404C, and 404D are equally spaced along the axial length of lead housing 402 at different axial positions. Each electrode level 404 may have one, two, three, or more electrodes located at different angular positions around the circumference (e.g., around the perimeter) of lead housing 402. As shown in FIG. 5A, electrode level 404A and 404D include a single respective ring electrode, and electrode levels 404B and 404C each include three electrodes at different circumferential positions. This electrode pattern may be referred to as a 1-3-3-1 lead in reference to the number of electrodes from the proximal end to the distal end of lead 400. Electrodes of one circumferential location may be lined up on an axis parallel to the longitudinal axis of lead 400. Alternatively, electrodes of different electrode levels may be staggered around the circumference of lead housing 402. In addition, lead 400 or 410 may include asymmetrical electrode locations around the circumference, or perimeter, of each lead or electrodes of the same level that have different sizes. These electrodes may include semi-circular electrodes that may or may not be circumferentially aligned between electrode levels.

Lead housing 402 may include a radiopaque stripe or other one or more radiopaque marker (not shown) along the outside of the lead housing. The radiopaque stripe corresponds to a certain circumferential location that allows lead 400 to be imaged and reliably localized when implanted in patient 112. Using the images of patient 112, the clinician can use the radiopaque stripe as a marker for the exact orientation of lead 400 within the brain of patient 112. Orientation of lead 400 may be needed to easily program the stimulation parameters by generating the correct electrode configuration to match the stimulation field defined by the clinician. In other embodiments, a marking mechanism other than a radiopaque stripe may be used to identify the orientation of lead 400. These marking mechanisms may include something similar to a tab, detent, or other structure on the outside of lead housing 402. In some embodiments, the clinician may note the position of markings along a lead wire during implantation to determine the orientation of lead 400 within patient 112. In some examples, programmer 104 may update the orientation of lead 400 in visualizations based on the movement of lead 400 from sensed signals.

FIG. 5B illustrates lead 410 that includes multiple electrodes at different respective circumferential positions at each of levels 414A-414D. Similar to lead 400, lead 410 is inserted through a burr hole, craniostomy, or craniotomy in cranium 122 to a target location within brain 120. Lead 410 includes lead housing 412. Four electrode levels 414 (414A-414D) are located at the distal end of lead 410. Each electrode level 414 is evenly spaced from the adjacent electrode level and includes two or more electrodes. In one embodiment, each electrode level 414 includes three, four, or more electrodes distributed around the circumference of lead housing 412. Therefore, lead 410 includes 414 electrodes in a preferred embodiment. Each electrode may be substantially rectangular in shape. Alternatively, the individual electrodes may have alternative shapes, e.g., circular, oval, triangular, rounded rectangles, or the like.

In alternative embodiments, electrode levels 404 or 414 are not evenly spaced along the longitudinal axis of the respective leads 400 and 410. For example, electrode levels 404C and 404D may be spaced approximately 3 millimeters (mm) apart while electrodes 404A and 404B are 10 mm apart. Variable spaced electrode levels may be useful in reaching target anatomical regions deep within brain 120 while avoiding potentially undesirable anatomical regions. The variable spacing may also be utilized to enhance the resulting AEF therapy generated between a pair of electrodes carried on any of leads 400 or 410. Further, the electrodes in adjacent levels need not be aligned in the direction as the longitudinal axis of the lead, and instead may be oriented diagonally with respect to the longitudinal axis.

Leads 400 and 410 are substantially rigid to prevent the implanted lead from varying from the expected lead shape. Leads 400 or 410 may be substantially cylindrical in shape. In other embodiments, leads 400 or 410 may be shaped differently than a cylinder. For example, the leads may include one or more curves to reach target anatomical regions of brain 120. In some embodiments, leads 400 or 410 may be similar to a flat paddle lead or a conformable lead shaped for patient 112. Also, in other embodiments, leads 400 and 410 may any of a variety of different polygonal cross sections (e.g., triangle, square, rectangle, octagonal, etc.) taken transverse to the longitudinal axis of the lead.

As shown in the example of lead 400, the plurality of electrodes of lead 400 includes a first set of three electrodes disposed at different respective positions around the longitudinal axis of the lead and at a first longitudinal position along the lead (e.g., electrode level 404B), a second set of three electrodes disposed at a second longitudinal position along the lead different than the first longitudinal position (e.g., electrode level 404C), and at least one ring electrode disposed at a third longitudinal position along the lead different than the first longitudinal position and the second longitudinal position (e.g., electrode level 404A and/or electrode level 404D). In some examples, electrode level 404D may be a bullet tip or cone shaped electrode that covers the distal end of lead 402.

FIGS. 5C-5F are transverse cross-sections of example stimulation leads having one or more electrodes around the circumference of the lead. As shown in FIGS. 5C-5F, one electrode level, such as one of electrode levels 404 and 414 of leads 400 and 410, are illustrated to show electrode placement around the perimeter, or around the longitudinal axis, of the lead. FIG. 5C shows electrode level 500 that includes circumferential electrode 502. Circumferential electrode 502 encircles the entire circumference of electrode level 500 and may be referred to as a ring electrode in some examples. Circumferential electrode 502 may be utilized as a cathode or anode as configured by the user interface. Any of the electrodes of FIGS. 5A-5F may be configured to act as a sensing electrode, or as part of a sensing electrode combination, within a tissue environment.

FIG. 5D shows electrode level 510 which includes two electrodes 512 and 514. Each electrode 512 and 514 wraps approximately 170 degrees around the circumference of electrode level 510. Spaces of approximately 10 degrees are located between electrodes 512 and 514 to prevent inadvertent coupling of electrical current between the electrodes. Smaller or larger spaces between electrodes (e.g., between 10 degrees and 30 degrees) may be provided in other examples. Each electrode 512 and 514 may be programmed to act as an anode or cathode.

FIG. 5E shows electrode level 520 which includes three equally sized electrodes 522, 524 and 526. Each electrode 522, 524 and 526 encompass approximately 110 degrees of the circumference of electrode level 520. Similar to electrode level 510, spaces of approximately 10 degrees separate electrodes 522, 524 and 526. Smaller or larger spaces between electrodes (e.g., between 10 degrees and 30 degrees) may be provided in other examples. Electrodes 522, 524 and 526 may be independently programmed as an anode or cathode for stimulation.

FIG. 5F shows electrode level 530 which includes four electrodes 532, 534, 536 and 538. Each electrode 532, 534, 536 and 538 covers approximately 80 degrees of the circumference with approximately 10 degrees of insulation space between adjacent electrodes. Smaller or larger spaces between electrodes (e.g., between 10 degrees and 30 degrees) may be provided in other examples. In other embodiments, up to ten or more electrodes may be included within an electrode level. In alternative embodiments, consecutive electrode levels of lead 114 may include a variety of electrode levels 500, 510, 520, and 530. For example, lead 114 (or any other lead described herein) may include electrode levels that alternate between electrode levels 510 and 530 depicted in FIGS. 5D and 5F. In this manner, various stimulation field shapes may be produced within brain 120 of patient 112. Further the above-described sizes of electrodes within an electrode level are merely examples, and the invention is not limited to the example electrode sizes.

Also, the insulation space, or non-electrode surface area, may be of any size. Generally, the insulation space is between approximately 1 degree and approximately 20 degrees. More specifically, the insulation space may be between approximately 5 and approximately 15 degrees. In other examples, insulation space may be between approximately 10 degrees and 30 degrees or larger. Smaller insulation spaces may allow a greater volume of tissue to be stimulated. In alternative embodiments, electrode size may be varied around the circumference of an electrode level. In addition, insulation spaces may vary in size as well. Such asymmetrical electrode levels may be used in leads implanted at tissues needing certain shaped stimulation fields. Although not shown, any lead or electrode array may include one or more fixation elements (e.g., tines, screws, electrode shapes, adhesives, etc.) that enable the lead or electrodes to be relatively fixed in position with respect to surrounding tissue.

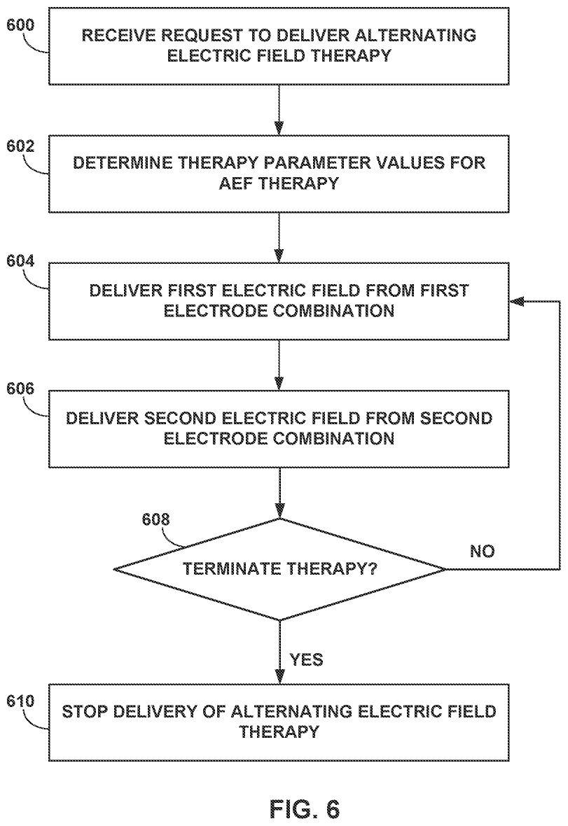

FIG. 6 is a flowchart illustrating an example technique for delivering AEF therapy to a patient. The technique of FIG. 6 will be described with respect to processing circuitry 210 of IMD 106 in FIG. 2. However, other processors, devices, or combinations thereof, such as processing circuitry 310 of programmer 104 or some combination of devices or processors, may perform the techniques of FIG. 6 in other examples. The technique of FIG. 6 may apply to therapies other than AEF therapy in a similar manner.

As shown in the example of FIG. 6, processing circuitry 210 receives a request to deliver alternating electric field (AEF) therapy (600), such as from external programmer 104 or a pre-programmed delivery schedule. Processing circuitry 210 then determines therapy parameter values for AEF therapy (602). This determination may be retrieval of parameter values from memory or determining one or more parameter values based on a delivery schedule, sensed data, or any other information.

Processing circuitry 210 then delivers the AEF therapy by delivering a first electric field from a first electrode combination (604) alternating with delivery of a second electric field from a second electrode combination different than the first electrode combination (606). In some examples, the first and second electrical fields may be phase shifted so as to not overlap. In other examples, the first and second electrical fields may be phase shifted so as to partially overlap in time. In some examples, the first and second electrical fields may be temporally interleaved to be fully non-overlapping or partially overlapping. Although the first and second electrical fields may be delivered with the same amplitude and frequency, the first and second electrical fields may be defined by different amplitudes and/or different frequencies (e.g., 150 kHz and 200 kHz). The first and second electrode combinations may use completely different electrodes or partially different electrodes, for example. The first and second electrode combinations may be selected to generate respective electrical fields that are orthogonal to each other or oblique, in some examples. Although all electrodes of the first and second electrode combinations may be implanted in some examples, one or more of the electrodes may be external electrodes in other examples. In general, processing circuitry 210 may determine the frequency of electrical field alternation based on the number of electrical combinations used to alternate the electrical fields for therapy. No interphase period may be required between the delivery of each electric field, although processing circuitry 210 may provide an interphase period in some examples.

Processing circuitry 210 then determines whether to terminate the AEF therapy (608). If processing circuitry 210 determines that AEF therapy is not to be terminated, processing circuitry 210 continues to deliver the first and second electric fields (604 and 606). If processing circuitry 210 determines that AEF therapy is to be terminated or otherwise paused, processing circuitry 210 stops delivering the AEF therapy to the patient (610).

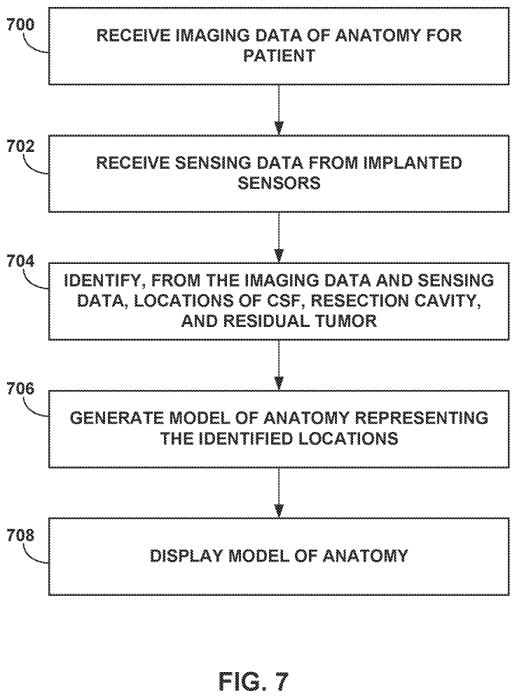

FIG. 7 is a flowchart illustrating an example technique for generating a model of anatomy for a patient for AEF therapy planning. The technique of FIG. 7 will be described with respect to processing circuitry 310 of programmer 104 in FIG. 3. However, other processors, devices, or combinations thereof, such as processing circuitry 210 of IMD 106 or some combination of devices or processors (e.g., server 130), may perform the techniques of FIG. 7 in other examples.

In the example of FIG. 7, processing circuitry 310 receives imaging data of anatomy for a patient (700). This imaging data may include at least one of data obtained by at least one of magnetic resonance imaging (MRI), computed tomography, or magnetoencephalography (MEG), or any other imaging modality. Processing circuitry 310 then receives sensing data from one or more implanted sensors in the patient (702). This sensed data may include LFPs, impedance tomography, voltage gradients or electric field strengths, or any other type of sensed information. Processing circuitry 310 may then identify, based on the imaging data and the sensing data, locations of cerebral spinal fluid, a resection cavity, and a possible residual tumor (704). In this manner, external and internal imaging techniques can be used to identify various tissue locations. These locations of anatomical structures and fluids may play a role in the propagation of electrical fields within the anatomy for a brain of a patient. Locations of other types of tissues may also be determined from the imaging data and/or sensing data. Different locations may be applicable to other types of target tissue. Based on the identified locations, processing circuitry 310 then generates a model of the anatomy for the patient (706). In some examples, the model of the anatomy may indicate locations of high electric field strength, high field gradients, or other electrical properties that may affect the AEF therapy. These high gradients may occur at edges or interfaces between different types of tissues such as between CSF and the resection bed.

Processing circuitry 310 can then output, for display, the model (708). For example, user interface 302 may generate a visual representation of the model to a user. In other examples, the model may be of a brain of the patient and identify various locations, target tissues, or other aspects of the anatomy for delivery of AEF therapy. A user, such as a surgeon, may utilize the model to plan implant locations for leads or electrodes and/or determine various stimulation parameters that will define AEF therapy.

For sensing data, the system may utilize an electrode array for electrical impedance tomography by injecting a current in a pair of electrodes and then recording the resulting voltage at other electrodes. The system can then generate a resulting reconstructed image using electrical impedance tomography. In some examples, additional electrodes may be utilized as floating ground during sensing in order to “soak” extra current delivered to the patient. These floating grounds may enable improved signal to noise ratio of the reconstructed image.

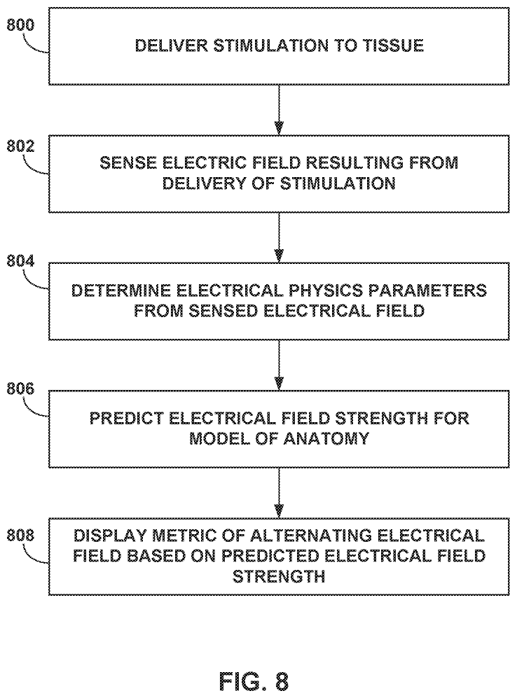

FIG. 8 is a flowchart illustrating an example technique for predicting electrical field strength for AEF therapy. The technique of FIG. 8 will be described with respect to processing circuitry 310 of programmer 104 in FIG. 3. However, other processors, devices, or combinations thereof, such as processing circuitry 210 of IMD 106 or some combination of devices or processors (e.g., server 130), may perform the techniques of FIG. 8 in other examples.

As shown in the example of FIG. 8, processing circuitry 310 is configured to control stimulation circuitry 208 of IMD 106 to deliver AEF therapy to patient 112 (800) and control sensing circuitry 204 to generate sensed data representative of a sensed electric signal resulting from delivery of the AEF therapy (802). Processing circuitry 310 then receives the sensed data and determines one or more electrical physics parameters indicative of the sensed electric field (e.g., the sensed electrical signal) (804) and predicts an electrical field strength for anatomy of the patient based on the electrical physics parameter, such as an electrical field strength (806). Then, processing circuitry 310 generates, based on an electrical field strength (or other electrical physics parameter), a metric of the AEF therapy and displays the metric of the AEF therapy (808).

In some examples, stimulation circuitry 208 is configured to deliver the AEF therapy via a first set of electrodes, and sensing circuitry 204 is configured to generate the sensed data from the sensed electrical signal obtained from a second set of electrodes different than the first set of electrodes. The sensing data may include at least one of evoked signals, local field potentials (LFPs), or impedance tomography, or measures of voltage gradient or electric field generated by the therapy. In some examples, processing circuitry 310 may predict the electric field strength over a volume of the anatomy. The metric may include a singular value indicative of the AEF therapy efficacy for a target tissue within the anatomy. In other examples, the metric may provide location specific information related to the predicted electrical field strength, such as gradients with respect to respective locations of the anatomy of the patient.

In this manner, a system can model electric field strength magnitude and/or direction across surrounding tissue and tumor volume. Multiple factors can impact this calculation, including: tissue conductivity for each tissue type, tissue relative permittivity for each tissue type, electrode selection, inter-electrode spacing, intra-electrode contact spacing, electrode contact size, proximity and distribution of fluid collections relative to the tissue of interest (i.e. CSF within sulci, ventricles, lesional cyst, or resection cavity), some of which may change over time with disease progress, recovery after surgery, or normal lifestyle and ageing effects. Therefore one, some, or all of these factors can be sensed and used to calculate the electric field strength. For example, data recording from stimulating parameters and resulting electric field reported by measurement electrodes can be used by the system to derive values for intrinsic electrical physics parameters (i.e. conductivity and relative permittivity). Following the derivation and/or assumption of one or more electrical physics parameters, the system can utilize a model of the patient anatomy to predict electrical field dispersion within the target organ environment (e.g., the brain). Programmer 104 or another device may display the metric indicative of alternating electrical field over tissue (as opposed to a static electric field of one polarity).

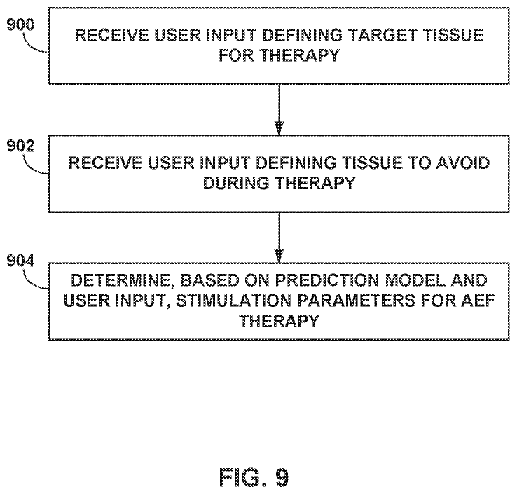

FIG. 9 is a flowchart illustrating an example technique for determining stimulation parameters for AEF therapy based on user input identifying target tissue. The technique of FIG. 9 will be described with respect to processing circuitry 310 of programmer 104 in FIG. 3. However, other processors, devices, or combinations thereof, such as processing circuitry 210 of IMD 106 or some combination of devices or processors (e.g., server 130), may perform the techniques of FIG. 9 in other examples.

As shown in the example of FIG. 9, processing circuitry 310 receives first user input defining target tissue to receive AEF therapy (900) and receives second user input defining tissue to avoid receiving AEF therapy (902). User interface 302 may receive the first and second user input. Then, processing circuitry 310 determines, based on at least one of the first user input or the second user input, one or more stimulation parameters that at least partially defines the AEF therapy (904). In other examples, processing circuitry 310 may only receive user input that defines the target tissue or only user input that identifies tissue to avoid.

Processing circuitry 310 can then control a medical device (e.g., IMD 106) to deliver the AEF therapy according to the one or more stimulation parameters. For example, programmer 104 can transmit the stimulation parameters to IMD 106. The one or more stimulation parameters may include one or more electrode combinations that at least partially define the AEF therapy. In addition, or alternatively, the one or more stimulation parameters comprises one or more implant locations for one or more leads that carry electrodes for delivering the AEF therapy.

In this manner, a user can define a desired target tissue and compare, or the system can automatically compare, predictions of electrical field to desired values for the anatomy. For treatment of glioblastoma, boundaries of the resection tumor bed (e.g., the region around surgically resected tumor) is the most likely target tissue for AEF therapy. In some examples, it may be beneficial for the user or system to define regions of tissue to avoid delivering alternating electric fields during therapy. These avoidance regions may be appropriate for regions with high conductivity in which current would be pulled and “wasted” within this tissue types (i.e. within the brain paths that go through CSF, such as the ventricle).

One possible approach for treating the patient with AEF is to provide weak electric fields spread over large area (in contrast to deep brain stimulation (DBS), which generally uses a more concentrated electrical signal). This relatively weaker electrical stimulation may avoid neural activation from electrical fields, enable the system to track levels of field strength as a function of activation threshold (e.g., sub- or supra-threshold stimulation), and track electrical fields. In some examples, programmer 104 can display a metric of this weaker electric field for real-time monitoring of AEF therapy by programmer 104 and/or the clinician. The metric may include dosimetry and/or may be used by IMD 106 in a closed-loop manner to maintain a minimum electrical field strength and/or prevent exceeding a maximum electrical field strength.

Despite a presumed minimal impact of thermal heating from implanted electrodes during AEF therapy, some tissue regions will be eloquent or particularly sensitive to the alternating electrical fields. Therefore, planning software provided by programmer 104 may include options for regional distinctions that identify acceptable level of risk. One example would be for cerebral implantation where the system can present eloquent regions of the brain to consider in avoiding neurological morbidity within the planning environment.

In some examples, predictive modeling may by employed by the system to update predictions of AEF magnitude and/or direction based on a new imaging evaluation of the patient's target organ of interest, such as the brain. The system may utilize predictive modeling to provide computer generated optimization of implantation strategy, such as the locations at which electrodes or leads should be implanted, to improve the ability of the system to treat a target region of interest (e.g., a tumor or resection bed) within the target organ and/or increase the coverage of therapy to the entire target organ. The system may generate predictions for initial implantation to aid in surgical planning and/or for updated or revised implantation configurations. Generally, predictions can accommodate or select different electrode styles (e.g., electrode grids, depth electrodes, etc.), different electrode pairing combinations, and different phase shifting pairs within different pairing combinations for various possible alternating electrode field options. In some example, each pair of electrodes (or electrode combination) may be phase shifted in order to deliver the alternating electric fields. Therefore, the system may utilize the magnitude of the phase shifting as part of predicting the AEF magnitude delivered to tissue.

These predictions can be leveraged by the system to determine appropriate wavelength and amplitude of the electrical signals applied. In some examples, the system may adjust options for reducing the number of leads, utilization of only certain types of leads, application of certain types of pairing combinations, and/or customization of all parameters within the dosimetry principles for AEF therapy. In some examples, the system may generate a target zone for standard treatment planning based on the target AEF dosimetry. This target zone may include the region of interest and some additional tissue width, such as 3 mm around resection cavity.

The system may perform modeling of tissue and predictions of AEF therapy at different times with respect to planning and delivery of therapy. In one example, the system may operate to provide preoperative modeling with predictive mapping of electrical fields anatomy to facilitate treatment planning. In some examples, the system may provide postoperative modeling with predictive mapping to guide treatment initial parameter selections for AEF therapy, such as frequencies, electrode combinations, amplitudes, and other parameter values. In addition, or alternatively, the system may generate progress update mapping following the acquisition of new imaging of the patient including the region of the previous implant. This process can enable the system and/or clinician to monitor for changes in residual tumor and the surrounding tissue, and account for remodeling of tissue that might occur post-surgery and/or normal changes with ageing or growth. User interface 302 of programmer 104 may display possible implant configuration and AEF dispersion predictions during treatment parameter programming. These user interface options may be different for clinicians and patients, such as providing more detailed parameter selection for clinicians and a simpler interface and selections for patients (e.g., on/off and/or amplitude adjustments.

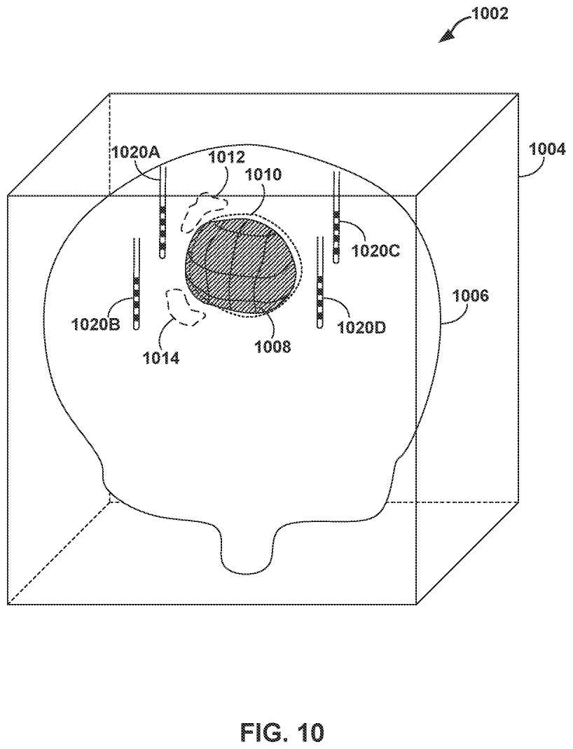

FIG. 10 is a conceptual diagram of an example three-dimensional user interface for programming AEF therapy. As shown in FIG. 10, user interface 1002 may be displayed as a three-dimensional representation (on a two-dimensional screen or a three-dimensional interface) of various elements associated with AEF therapy. User interface 1002 provides three-dimensional environment 1004 that includes brain 1006, leads 1020A, 1020B, 1020C, and 1020D (collectively “leads 1020”). Affected tissue 1008 indicates the tissue region that receives AEF therapy to a level that can treat the cells within, target tissue 1010 is the tissue area defined by user input that is desired to be treated, and avoidance regions 1012 and 1014 are those tissue areas that the user input has defined for avoiding AEF therapy.

User interface 1002 can be used as an AEF therapy planning tool (e.g., for lead implantation locations and other stimulation parameters that define AEF therapy) and as a tool for updating stimulation parameters after therapy has begun. For example, a user may initially provide user input defining target tissue 1010 and/or avoidance regions 1012 and 1014 (or more). Brain 1006 may include imaging data that represents various locations of different tissues and structures within the anatomy so that the user can identify which regions of interest for AEF therapy. The user input may be input outlining each tissue region, dragging outlines to desired locations, moving regions to a desired location, or any other input method. In some examples, the system may automatically suggest target tissue 1010, but the user can move or adjust that region as desired.

Based on the target tissue 1010 and/or avoidance regions 1012 and 1014, the system may recommend a certain number of leads 1020, implant locations for leads 1020, types of leads 1020, any other hardware recommendations. The system may also, or alternatively, recommend stimulation parameter values that will result in affected tissue 1008 that may be similar to target tissue 1010 while reducing the overlap with any of avoidance regions 1012 and 1014. User interface 1002 may receive user input moving one or more of leads 1020, adjusting one or more stimulation parameters, and/or adjusting one or more of target tissue 1010 or any of avoidance regions 1012 and 1014. In some examples, user interface 1002 may also show additional features related to AEF therapy, such as electrical field gradients. As described herein, the system may include a therapy planning module that generates recommendations for one or more aspects of AEF therapy (or other therapy) based on user input and/or sensed signals associated with the patient. The system may generate recommendations for lead placement, electrode selection, or other stimulation parameters based on any available information for the patient and predictions generated using the patient model or other characteristics. In some examples, the therapy planning module may provide guidance that may reduce electric field spread into unwanted or avoided regions which can increase energy efficiency for therapy and/or reduce unwanted side effects from the delivered electric field therapy. For example, since AEF therapy may reduce growth of cancerous tissue, electric field spread into healthy tissue may also reduce the growth or other function of cells in the healthy tissue.

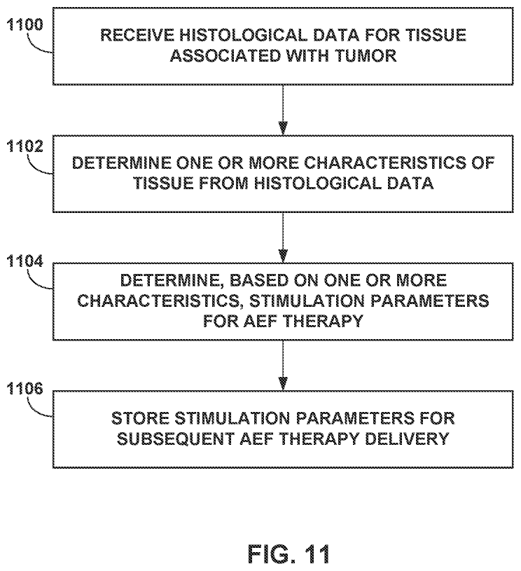

FIG. 11 is a flowchart illustrating an example technique for determining stimulation parameters for AEF therapy based on histological data from tumor tissue. The technique of FIG. 11 will be described with respect to processing circuitry 310 of programmer 104 in FIG. 3. However, other processors, devices, or combinations thereof, such as processing circuitry 210 of IMD 106 or some combination of devices or processors (e.g., server 130), may perform the techniques of FIG. 11 in other examples.

In the example of FIG. 11, processing circuitry 310 can receive histological data for tissue associated with a glioblastoma tumor (1100). For example, a clinician may take a sample of tissue from the patient and perform one or more histological analyses on the sample. Then, processing circuitry 310 or a user can determine one or more characteristics of tissue from the histological data (1102). Based on these one or more characteristics, processing circuitry 310 can determine stimulation parameters for AEF therapy (1104). For example, processing circuitry 310 can make an adjustment to one or more parameters to refine the AEF therapy, either retrospectively using stored data or in real-time as sensed data is received. Processing circuitry 310 can then store the new stimulation parameters for subsequent AEF therapy delivery (1106) and, in some cases, control IMD 106 to deliver the updated AEF therapy.

Given that cells respond differently to AEF therapy largely due to differences in the physical size and orientation of different tumor cells, these personalized stimulation parameters can be more accurately assigned following histological analysis of a given patient's tumor biopsy. Cell size can be an important physical parameter because the cell size impacts the dispersion of electric field across the cell in a predictable pattern. In some examples, the system may select lower frequency of AEF therapy for larger cells than smaller cells. In this manner, a patient-specific therapy plan can include customized stimulation parameters such as AEF magnitude or strength, frequency, vectoral field direction, phase shift, etc. In some examples, the system or user can utilize a patient-specific tumor cell culture to establish patient-specific treatment parameters that increase the cells inhibition of mitotic activity. For example, an amplitude of 2.25V/cm or a frequency of 187.3 kHz may be used as an AEF configuration to cease cell division in target tissue. This magnitude is likely variable dependent on the tumor type, patient-specific tumor subtype, and other treatment parameters (e.g., AEF frequency). Other parameters that may be adjusted based on patient-specific cell status may include duty cycle, frequency, amplitude, and duration of exposure to the AEF therapy.

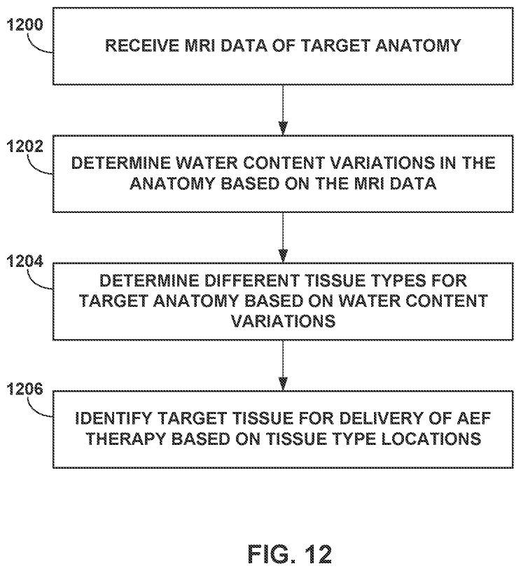

FIG. 12 is a flowchart illustrating an example technique for identifying target tissue for AEF therapy based on water content determined from imaging data. The technique of FIG. 12 will be described with respect to processing circuitry 310 of programmer 104 in FIG. 3. However, other processors, devices, or combinations thereof, such as processing circuitry 210 of IMD 106 or some combination of devices or processors (e.g., server 130), may perform the techniques of FIG. 12 in other examples.

In the example of FIG. 12, processing circuitry 310 can receive MRI data for target anatomy associated with a glioblastoma tumor (1200). Then, processing circuitry 310 or a user can determine water content variations in the target anatomy based on the MRI or diffusion tensor imaging (DTI) data (1202). Based on the water content variations, processing circuitry 310 can determine different tissue types for the target anatomy (1204). The water content variations may be indicative of conductivity estimates for the tissue. In some examples, water content variations may be indicative of tissue boundaries. Then processing circuitry 310 can identify target tissue for delivery of AEF therapy based on the locations of the tissue types (1206). In some examples, processing circuitry 310 can determine stimulation parameters for AEF therapy in order to deliver the AEF therapy to the target tissue. Processing circuitry 310 can then store the stimulation parameters for subsequent AEF therapy delivery (1106) and, in some cases, control IMD 106 to deliver the updated AEF therapy.

In this manner, modeling of AEF therapy can be informed by assumptions regarding the electrophysical parameters of tissue types. The system can then utilize MRI data to determine one or more physical parameters based on the water content (and/or boundaries between different tissue types) quantified by the imaging, e.g., MR electrical properties tomography (MREPT). The water content data can serve as a baseline for model generation which can be enhanced further utilizing real-time patient data from implanted recording, which may leverage machine learning techniques. Existing efforts to perform modeling of body tissues (particularly the brain) are challenged by the anatomical complexity and person-to-person variability. Therefore, assumptions regarding electrophysical parameters can reduce modeling accuracy considerably. Measuring water content and other aspects can increase the modeling accuracy. In some examples, the water content data can improve conductivity estimates for known tissue structures within the brain of the patient. In addition, or alternatively, the system may segment the anatomy based on the water content to indicate regions with different electrical conductivity.

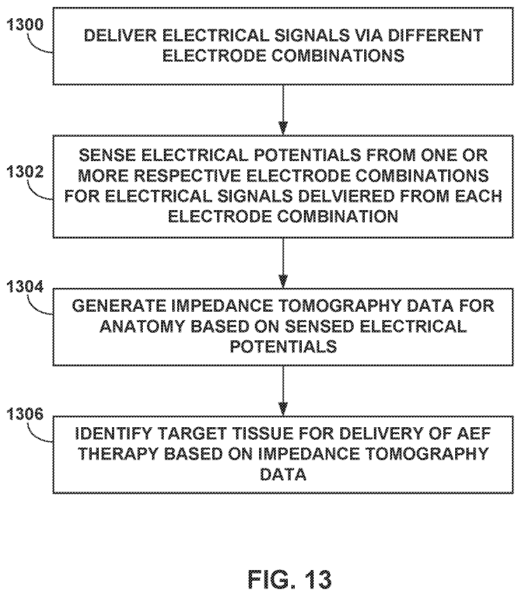

FIG. 13 is a flowchart illustrating an example technique for identifying target tissue based on impedance tomography data. The technique of FIG. 13 will be described with respect to processing circuitry 310 of programmer 104 in FIG. 3. However, other processors, devices, or combinations thereof, such as processing circuitry 210 of IMD 106 or some combination of devices or processors (e.g., server 130), may perform the techniques of FIG. 13 in other examples.

In the example of FIG. 13, processing circuitry 310 controls IMD 106 to deliver electrical signals via different electrode combinations (1300). IMD 106 then senses electrical potentials from one or more respective electrode combinations for electrical signals delivered from each electrode combination (1302). Then, processing circuitry 310 can receive the sensed electrical potentials and generate impedance tomography data for the patient anatomy based on the sensed electrical potentials (1304). Based on the impedance tomography data, processing circuitry 310 can identify target tissue for delivery of AEF therapy (1306). In other examples, IMD 106 may perform some or all of these steps in order to identify target tissue based on the impedance tomography data.

As discussed herein, the impedance tomography data can characterize types of tissue within the treatment planning environment for AEF therapy. The electrical potentials sensed from the anatomy can be used to determine impedance measures to localize the area of the glioblastoma progression or likelihood of progression. Impedance tomography can characterize the heterogeneity of the cells in the anatomy, which can improve understanding of the tissue subject to the AEF therapy. For example, the system or user can utilize the impedance tomography data in order to identify the greatest density of tumor cells, which may be identified as at least part of the target tissue for AEF therapy, which can inform lead placement.

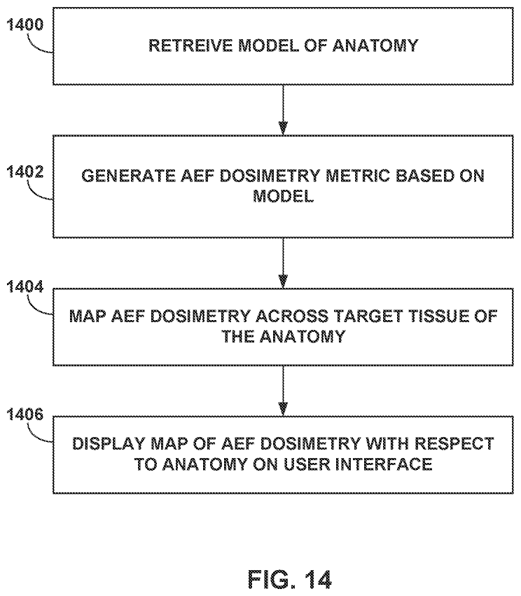

FIG. 14 is a flowchart illustrating an example technique for generating a map of AEF dosimetry for a patient. The technique of FIG. 14 will be described with respect to processing circuitry 310 of programmer 104 in FIG. 3. However, other processors, devices, or combinations thereof, such as processing circuitry 210 of IMP 106 or some combination of devices or processors (e.g., server 130), may perform the techniques of FIG. 14 in other examples.

In the example of FIG. 14, processing circuitry 310 can receive a model of the anatomy of the patient (1400). This model may be generated based on one or more imaging modalities as described herein. Then, processing circuitry 310 can generate an AEF dosimetry metric based on the model (1402). This dosimetry metric may represent the effective therapy dose of the AEF received by cells during therapy. Processing circuitry 310 can then map the AEF dosimetry across the target tissue of the anatomy in order to indicate which areas of tissue are receiving different doses of AEF therapy (1404). In order to visualize the map, processing circuitry 310 may control user interface 302 to display the map of the AEF dosimetry with respect to the anatomy (1406). For example, the user can identify which regions of tissue may be receiving a too high dose, or a dose that is insufficient for therapy.

As discussed herein, characteristics of the AEF therapy that may influence effectiveness include frequency, magnitude, and vectoral direction of the electrical fields. One dosimetry metric may be an average field intensity. The average field intensity may instead be referred to as a local minimum field intensity (LMiFI). For example, this metric may be equivalent to a lower of the 2-field intensities from 2 orthogonal directions used during AEF treatment. In situations in which the electrode combinations provide more than 2 orthogonal directions, this metric may represent an average of all AEF magnitudes experienced by a single point in space over a period of time, such as a one second interval.

Another dosimetry metric may be referred to as the power loss density. The power loss density may be referred to as the energy per unit of time deposited by AEF therapy, which may be P=½σE². P is the power loss density (e.g., Watt/volume), a is the tissue conductivity (Siemens/meter), and E is the magnitude of the electric field (Volts/cm). In some examples, the average field intensity metric could be used in place of the magnitude of the electric field to provide a combination of information that may indicate the power dispersion over the tissue. Another dosimetry metric may be a dose density which can be referred to as local minimum dose density. The dose density metric may be equivalent to the local minimum power density (LMiPD) multiplied by the average patient compliance with treatment. In other words, if the patient does not comply with the treatment schedule, the dose density metric decreases. AEF therapy using IMD 106 may increase patient compliance to, or close to, 100% because the patient can receive therapy during most times of the day. However, less compliance may be relevant and used to identify reduced dosages that may affect therapy efficacy.

Any of the AEF dosimetry metrics can be displayed by the user interface of the modeling predictions for mapping AEF across a target tissue of interest. For a treatment planning system, the dosimetry metric can be used to specify the desired region of effect. For example, the user interface may present the dosimetry metric at various stages of planning in order to inform stimulation parameter selection, lead implant locations, durations of therapy, amplitudes, frequencies, or any other parameters. In some examples, the system may display a target dosimetry that the user, or system, may attempt to achieve for the target tissue during therapy planning. In some examples, mapping the AEF therapy may utilize the power loss density metric in order to distinguish between various electrode locations, electrode combinations, or other stimulation parameters during treatment planning. In some examples, dosimetry metrics may account for phase shifting if not already addressed in the metric. In some examples, the user will be configured to receive one or more of user selection of a defined target tissue 1010, user selection of one or more lead style(s), user selection of lead locations, user selection of lead stimulation amplitudes, or user selection of lead stimulation frequencies. For any of these parameters that the system does not receive user input, the system may automatically select parameter values that would be appropriate to satisfy the given user input received. In some examples, the system can then output appropriate AEF dosimetry metrics resulting within the 1010 target tissue environment. Example user input fields that can be used to receive user input may include text boxes, magnitude or location sliders, numerical input, drop down selection menus, (e.g., lead styles or lead location), or any other type of input mechanism.

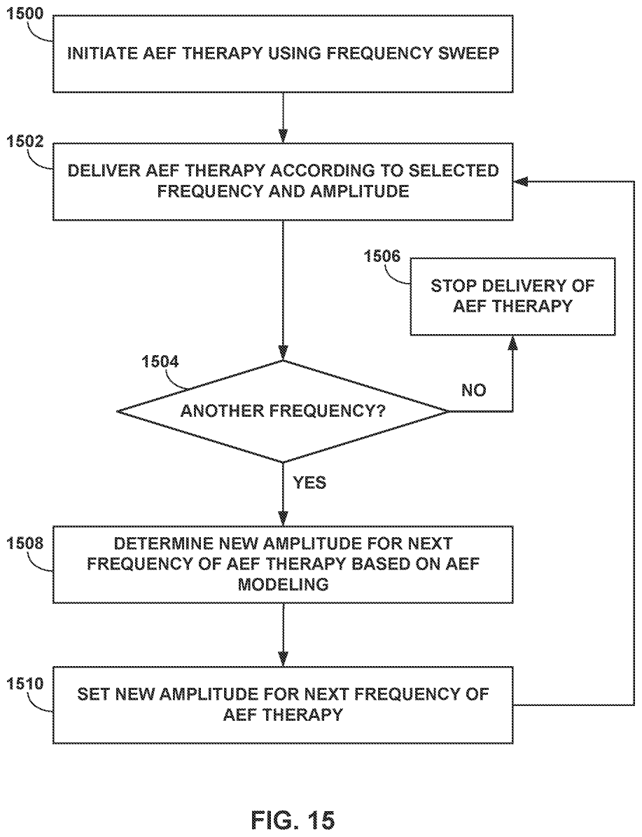

FIG. 15 is a flowchart illustrating an example technique for sweeping through different frequencies for AEF therapy. The technique of FIG. 15 will be described with respect to processing circuitry 210 of IMD 106 in FIG. 2. However, other processors, devices, or combinations thereof, such as processing circuitry 310 of programmer 104 or some combination of devices or processors (e.g., server 130), may perform the techniques of FIG. 15 in other examples.

In the example of FIG. 15, processing circuitry 210 initiates AEF therapy using a frequency sweep (1500). First, processing circuitry 210 controls IMD 106 to deliver the AEF therapy according to a selected frequency and amplitude (1502) for a predetermined period of time. This selected frequency may be one of several frequencies part of the sweep. If processing circuitry 210 determines there are no other frequencies to deliver (“NO” branch of block 1504), processing circuitry 210 stops delivery of the AEF therapy (1506). If processing circuitry 210 determines that there is another frequency to use (“YES” branch of block 1504), processing circuitry 210 determines the new frequency and the new amplitude for that frequency of AEF therapy based on AEF modeling (1508). Since the electrical field propagation through tissue changes with frequency, processing circuitry 210 may adjust the amplitude of stimulation for different frequencies. Processing circuitry 210 then sets the new amplitude for the next frequency of AEF therapy (1510). In some examples, the amplitude selections for respective frequencies may be performed prior to therapy and stored for later retrieval as different sets of parameters for each frequency.

As discussed above, each AEF frequency permeates tissue types differently. Therefore this modeling variable can be informed by real-time clinical application data recording to estimate the electrical physics parameters (e.g., impedance and/or permeability) relevant to the tissue type at a given AEF frequency. In use, the system can ensure constant field strength from AEF therapy as IMD 106 changes frequency by changing voltage amplitudes to account for changing tissue permeabilities during the frequency sweep. In some examples, the amplitude selections may be based on generalized tissue data or patient populations. In other examples, the patient specific model of tissue may be used to select amplitudes for respective frequencies. The utilization of measurement electrodes can enable data acquisition during real-time clinical delivery of AEF from implanted electrodes. The system can then utilize the measurements for improving the model of tissue under AEF therapy. This improved model can then be leveraged by the system to improve modeling predictions during implant planning and or delivery of therapy.

As described above, processing circuitry, such as processing circuitry 210, can receive a request to deliver alternating electric field (AEF) therapy, wherein the AEF therapy comprises delivery of a first electric field and a second electric field. Processing circuitry 210 can also control an IMD 106 to deliver the AEF therapy by iteratively sweeping through each selected frequency of a plurality of frequencies, where, for each selected frequency of the plurality of frequencies, processing circuitry 210 controls IMD 106 to deliver the first electric field from a first electrode combination of implanted electrodes at the selected frequency in alternating fashion with the second electric field from a second electrode combination of implanted electrodes different than the first electrode combination and at the selected frequency. In addition, processing circuitry 210 or other device can determine respective amplitudes for each selected frequency of the plurality of frequencies based on a model of AEF therapy.

In some examples, the system can predict progression zones, which may be areas of tissue where disease progression may occur, such as progression within the brain (for glioblastoma). Therefore, this prediction of progression can be combined with the AEF treatment modeling environment to further establish target tissue or other parameters via the user interface for the clinician during procedural planning and treatment parameter evaluations. In some examples, clinical data research can be conducted utilizing data acquisition of regional recurrence relative to the AEF treatment dosimetry map in populations in order to refine AEF therapy over time.

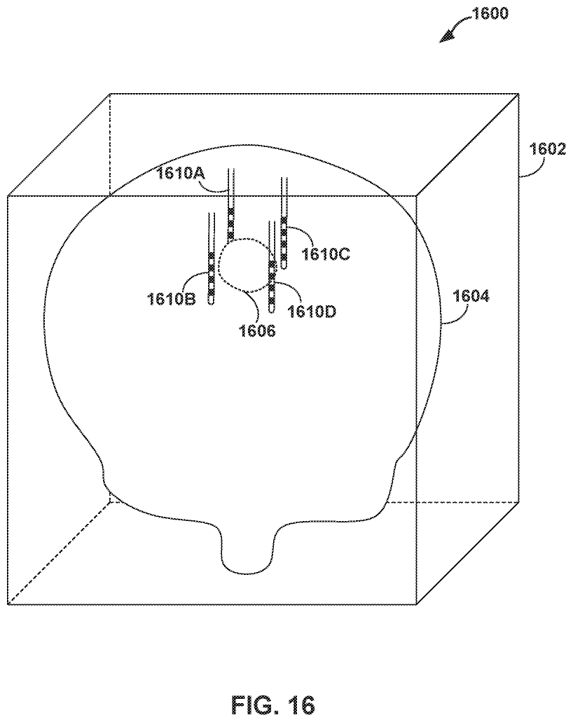

FIG. 16 is a conceptual diagram of an example three-dimensional user interface 1602 for planning implantation of electrodes for AEF therapy. As shown in FIG. 16, user interface 1600 may be displayed as a three-dimensional representation (on a two-dimensional screen or a three-dimensional interface) of various elements associated with AEF therapy. User interface 1600 provides three-dimensional environment 1602 that includes brain 1604, leads 1610A, 1610B, 1610C, and 1610D (collectively “leads 1610”). Resection bed 1606 indicates the location of the area from which a glioblastoma tumor was removed. Initially, imaging data may be used to identify the location, size, and shape of resection bed 1606 with respect to other tissues or structures (not shown) identified within brain 1604.

Then, the system may automatically place one or more leads 1610 in spatial relation to resection bed 1606 in order to treat that area, such as at the boundary of resection bed 1606 and adjacent unremoved tissue. User interface 1600 may receive user input moving any of leads 1610 to a different location. The locations of leads 1610 may be used to plan implant locations for the patient. The electrodes of leads 1610 may be used to deliver AEF therapy and/or sense signals. For example, sensing electrodes at this boundary of resection bed 1606 may enable the system to measure AEF dosimetry metrics.

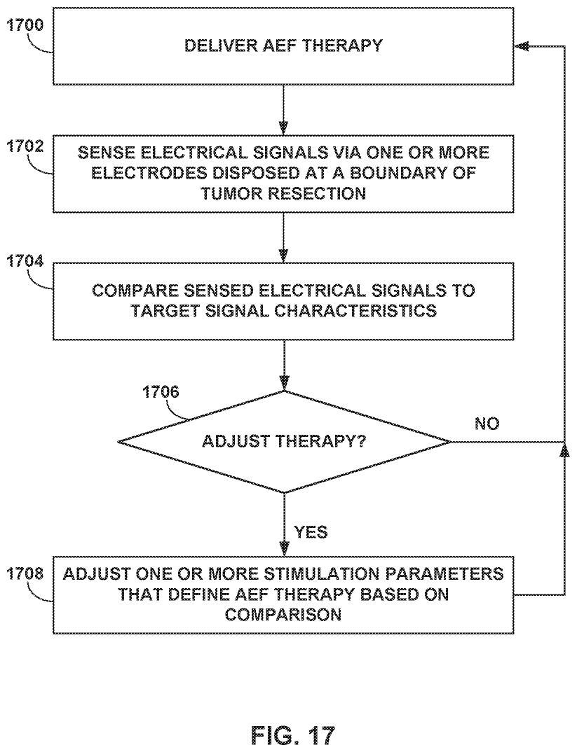

FIG. 17 is a flowchart illustrating an example technique for adjusting stimulation parameters for AEF therapy based on sensed electrical signals. The technique of FIG. 17 will be described with respect to processing circuitry 210 of IMD 106 in FIG. 2. However, other processors, devices, or combinations thereof, such as processing circuitry 310 of programmer 104 or some combination of devices or processors (e.g., server 130), may perform the techniques of FIG. 17 in other examples.

In the example of FIG. 17, processing circuitry 210 delivers AEF therapy (1700). Processing circuitry 210 then controls IMD 106 to sense electrical signals via one or more electrodes disposed at a boundary of a tumor resection bed (1702), such as shown in FIG. 16. Then, processing circuitry 210 compares the sensed electrical signals to target signal characteristics (1704). This process may enable the system to adjust AEF therapy based on one or more feedback variables to increase therapy efficacy.

If processing circuitry 210 determines therapy should not be adjusted (“NO” branch of block 1706), processing circuitry 210 continues to deliver AEF therapy (1700). If processing circuitry 210 determines that adjustments should be made (“YES” branch of block 1706), processing circuitry 210 adjusts one or more stimulation parameters that define AEF therapy based on the comparison. The process then continues to deliver AEF therapy with the adjusted parameters. In some examples, processing circuitry 210 may adjust the parameters within (e.g., limited by) one or more safety ranges, minimum levels, or energy budgets. In this manner, the adjustment may be limited in one or more respects. For example, processing circuitry 210 may deliver as much energy as possible until a recharge interval hits a low patient tolerated value.

In this manner, IMD 106 may operate such that stimulation circuitry 202 is configured to deliver AEF therapy to a patient and sensing circuitry 204 is configured to generate sensed data representative of a sensed electric signal(s) via one or more electrodes disposed at a boundary of a tumor resection. Processing circuitry 210 can then receive the sensed data, compare the sensed data to one or more target signal characteristics, adjust, based on the comparison, one or more stimulation parameters from a first value to a second value, and control the stimulation circuitry to deliver the AEF therapy according to the second value of the one or more stimulation parameters. In some examples, processing circuitry 210 is configured to compare the sensed data to one or more target signal characteristics at a predetermined interval. The stimulation parameters may include one or more electrode combinations, a frequency, or a cycling duration for the AEF therapy.

In some examples, processing circuitry 210 or other device may modulate AEF dosimetry parameters to maintain therapeutic effect based on the measured electrode feedback (e.g., signals sensed at the sensing electrodes). The sensed parameters may include amplitude (Vpp), electrode pairing (e.g., electrode combination and/or polarity), and AEF frequency. Processing circuitry 210 may initiate sensing of electrical signals are various times, such as following implantation to establish tissue baseline for initial treatment parameter selection or on continuous or interval based schedules over time to account for changes in target tissue (e.g., scarring, tumor progression) or lead migration.

In some examples, the system may utilize different stimulating and measuring electrodes to determine electrical field at target region around or within the tumor or resection bed. Processing circuitry 210 may time interleave stimulating and sensing functions. Further, one or more extra electrodes may be configured to “soak” up stimulation (like floating ground to work with stimulating and sensing electrodes) during sensing functionality. These current sink electrodes may be selected based on where the sensing electrodes are and/or where the target tissue is located with respect to the electrodes.

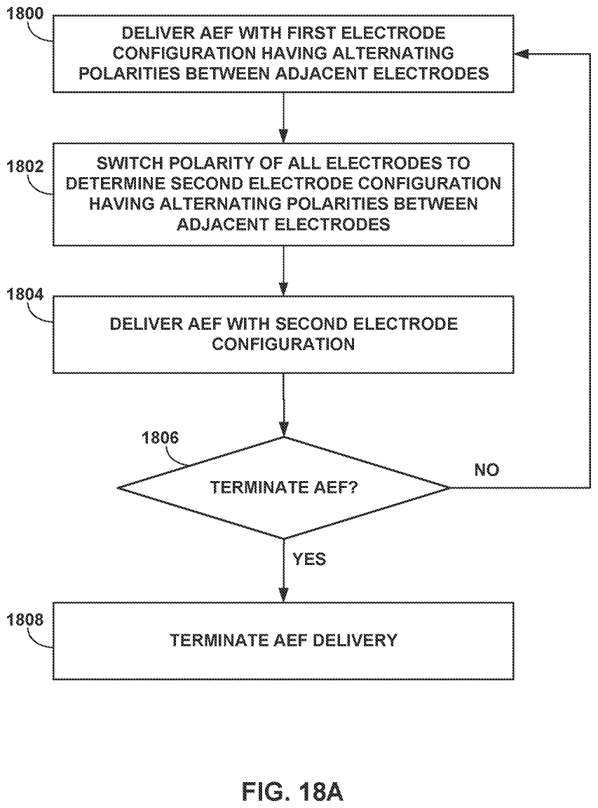

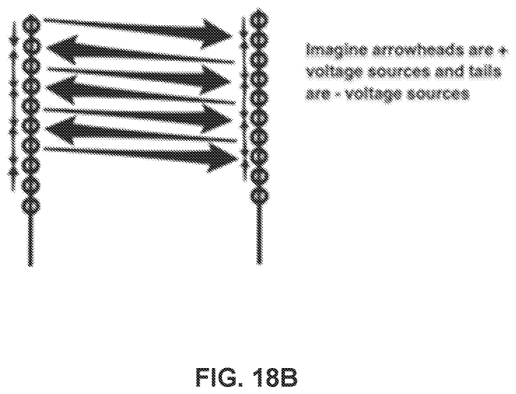

FIG. 18A is a flowchart illustrating an example technique for switching polarity of electrodes for AEF therapy. The technique of FIG. 18A will be described with respect to processing circuitry 210 of IMD 106 in FIG. 2. However, other processors, devices, or combinations thereof, such as processing circuitry 310 of programmer 104 or some combination of devices or processors (e.g., server 130), may perform the techniques of FIG. 18 in other examples.

In the example of FIG. 18A, processing circuitry 210 delivers AEF therapy with a first electrode configuration having alternating polarities between adjacent electrodes (1800). Processing circuitry 210 then controls IMD 106 to switch polarity of all electrodes to determine second electrode configuration having alternating polarities between adjacent electrodes (1802). In some examples, this switching may be between more than two electrode configurations. The number of electrode configurations may only be limited by the possible electrode combinations available to IMD 106. This switching scheme is shown in FIG. 18B. Then, processing circuitry 210 delivers AEF therapy with the second electrode configuration (1804). If processing circuitry 210 determines therapy should not be terminated (“NO” branch of block 1806), processing circuitry 210 continues to deliver AEF therapy (1800). If processing circuitry 210 determines that therapy is to be terminated (“YES” branch of block 1806), processing circuitry 210 terminates or stops AEF therapy (1808).

FIG. 18B is a conceptual drawing illustrating an example progression of polarity switching for AEF therapy. As shown in FIG. 18B, the vectoral direction of the electric field is ideally parallel to the axis of cell division designed to hinder the mitotic process of cells. The arrow heads represent anodes and the arrow tails indicate cathodes.

The chain strategy for electrode contact pairing shown in FIGS. 18A and 18B illustrate that pairing is defined as a state of inverse waveform state comparing one contact to the other. Within the chain adjacent electrode contacts are 180 degrees (1π) out of phase between electrodes resulting in increased change in voltage between the tissue separating the two given electrode contacts. This may result in the strongest electric field magnitude. Pairing of electrode contacts with separate adjacent electrodes can permit greater control of the electric field vectoral direction. Therefore a vast number of potential directional options can be dependent on the selected pairing, phase shifting, and inter-lead 3-dimensional configuration.

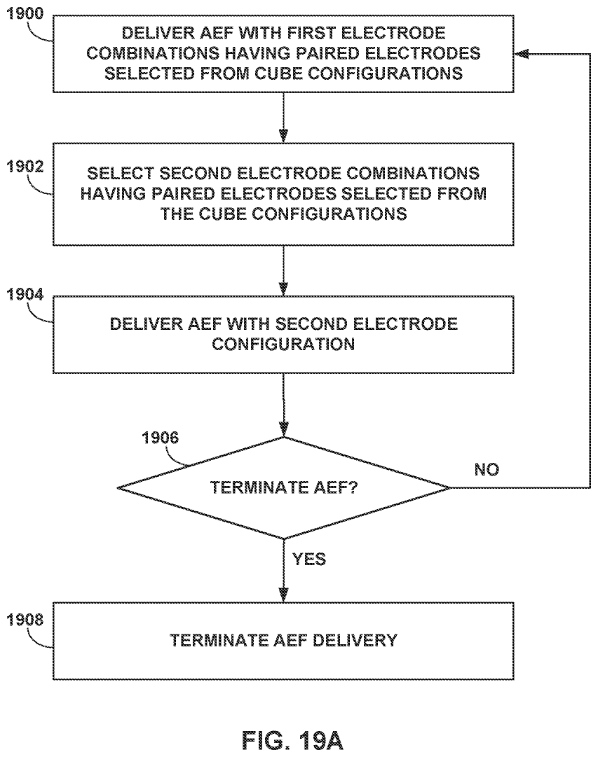

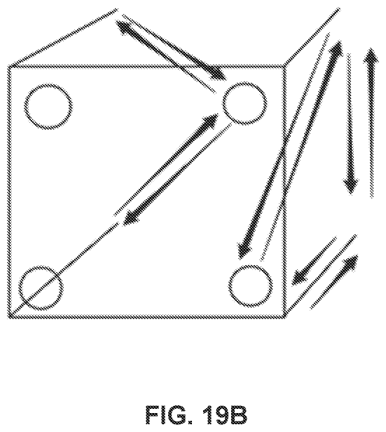

FIG. 19A is a flowchart illustrating an example technique for switching polarity between electrode pairs of a cube configuration for AEF therapy. The technique of FIG. 19A will be described with respect to processing circuitry 210 of IMD 106 in FIG. 2. However, other processors, devices, or combinations thereof, such as processing circuitry 310 of programmer 104 or some combination of devices or processors (e.g., server 130), may perform the techniques of FIG. 19A in other examples.

In the example of FIG. 19A, processing circuitry 210 delivers AEF therapy with a first electrode configuration having paired electrodes selected from cube configurations (1900). Processing circuitry 210 then controls IMD 106 to select second electrode combinations having paired electrodes selected from the cube configurations (1902). This switching scheme is shown in FIG. 19B. Then, processing circuitry 210 delivers AEF therapy with the second electrode configuration (1904). If processing circuitry 210 determines therapy should not be terminated (“NO” branch of block 1906), processing circuitry 210 continues to deliver AEF therapy (1900). If processing circuitry 210 determines that therapy is to be terminated (“YES” branch of block 1906), processing circuitry 210 terminates or stops AEF therapy (1908).

FIG. 19B is a conceptual drawing illustrating an example progression of paired electrode selection using cube configurations for AEF therapy. As shown in FIG. 19B, the cube strategy for electrode contact pairing includes pairing defined as a state of inverse waveform state comparing one contact to the other. Within the cube configuration, pairing is performed between contacts along the 8 apices of the cube. This provides for 28 possible electrode pairing combinations and therefore numerous potential methods of achieving vectoral electric field control within the region of the cube. In one examples, IMD 106 may include independent voltage or current sources to enable IMD 106 to incrementally sweep the field vector from an apex on one cube to another (e.g., from one electrode to another electrode in an electrode array). In this manner, IMD 106 may provide more intermediate field orientations that can impact cells.

Over time, the system can vary between different electrodes at different times to sweep polarity and field strength over the entire volume of target tissue. This method may result in complex steering with changing direction, phase, and field strength to achieve greater control of electric field vectoral direction and strength for AEF therapy.

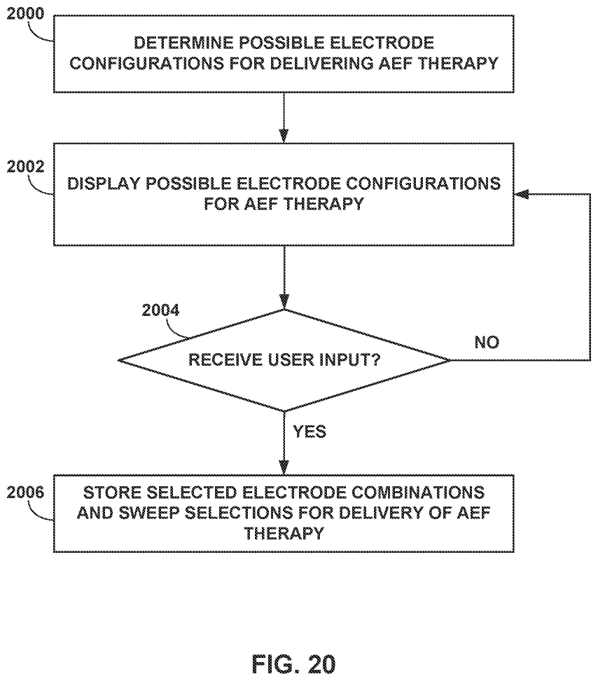

FIG. 20 is a flowchart illustrating an example technique for displaying user selectable electrode configurations for AEF therapy. The technique of FIG. 20 will be described with respect to processing circuitry 310 of programmer 104 in FIG. 3. However, other processors, devices, or combinations thereof, such as processing circuitry 210 of IMD 106 or some combination of devices or processors (e.g., server 130), may perform the techniques of FIG. 20 in other examples.

In the example of FIG. 20, processing circuitry 310 determines possible electrode combinations for delivering AEF therapy (2000). These electrode combinations may be used for sweeping AEF therapy between different electrode combinations which may reach additional tissue. Then, processing circuitry 310 displays the possible electrode configurations for AEF therapy (2002). User input may also select a desired order in which the electrode combinations may be used to deliver AEF therapy. In some examples, processing circuitry 310 may preselect a subset of recommended electrode combinations based on already collected data such as target tissue locations. If processing circuitry 210 determines that user input was not received (“NO” branch of block 2004), processing circuitry 210 continues to present the possible electrode combinations (2002). If processing circuitry 210 determines that user input was received (“YES” branch of block 2004), processing circuitry 210 stores the selected electrode combinations and sweep selections for delivery of AEF therapy (2006).

In some examples, the system may map different stimulation channels (e.g., electrode configurations) using multiple contacts can be more complex than 1:1 mapping. This may enable greater control of AEF dosimetry with a given implantation configuration. Since different electrode configurations can provide different orientation phasing when everything changes, the system may provide a disruptive environment within the tissue between the two paired stimulating electrode contacts. Configurations of stimulation channels can be wired for different directional pairing (i.e. diagonal vs. inline). The system may sweep through configurations to get many different permutations to cover the field. In some examples, the system may provide many independently controlled electrodes (e.g., 128 or more electrodes) to enable any pairing configurations an option at any point in time to cover target tissue and/or avoid other tissue.

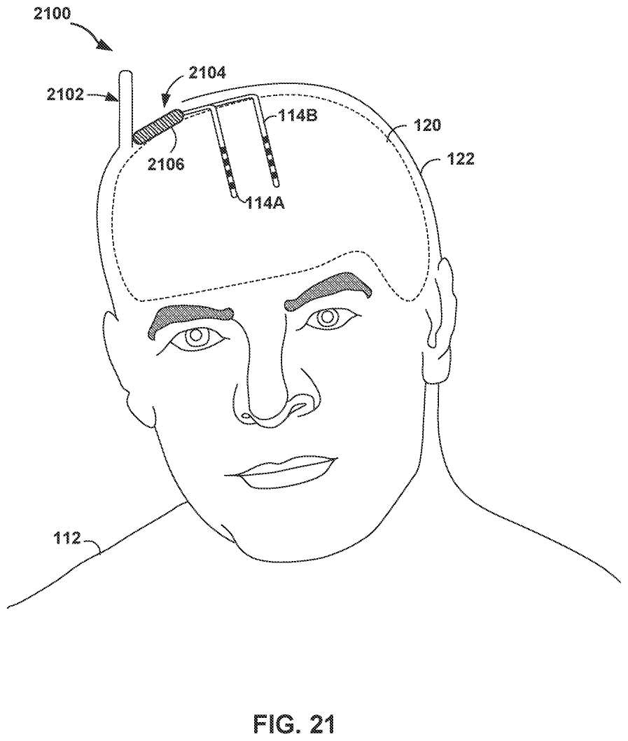

FIG. 21 is a conceptual diagram illustrating an example implantable medical device for delivering AEF therapy. As shown in FIG. 21, system 2100 includes IMD 2106 connected to leads 114A and 114B carrying respective electrodes. IMD 2106 may be referred to as a cranioplasty implant. Following craniotomy for tumor resection from brain 120 of patient 112, bone flap 2102 is still flipped out from the rest of cranium 122. Then, IMD 2106 could be installed within the bone defect (i.e. craniotomy/craniectomy) 2104 (or burr hole or craniostomy in cranium 122) or within in the bone flap 2102 itself.

The benefit of this implantation approach is the ability to get an implant within the intracranial space without impinging on the cerebrum. Although IMD 2106 may be further from the target zone (e.g., the resection bed), leads 114A and 114B coupled to ID 2106 may be inserted to the target tissue. In some examples, leaving the dura open may enable the implant at the level of the cranioplasty to act within the subdural space without impedance. In some examples, leads 114A and 114B may provide an option to remain within the resection cavity or penetrate the cerebral surface, to serve as a surveillance method for the resection bed. In other examples, if the resection bed is closer to the surface of brain 122, electrodes may be carried on IMD 2106 housing itself instead or addition to leads 114A and 114B. Fewer or more leads may be used in other examples, inclusive examples in which multiple cranioplasty implants 2106 are inserted within the same cranium 122.

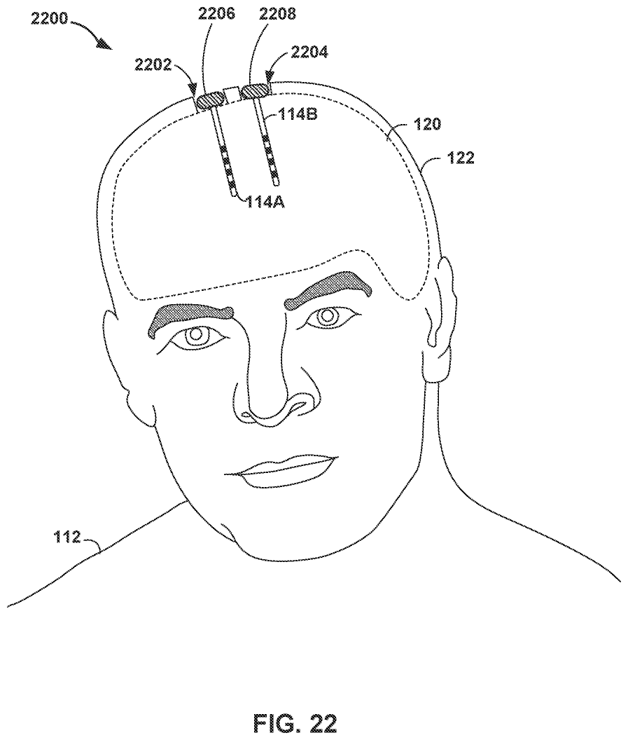

FIG. 22 is a conceptual diagram illustrating example implantable medical devices disposed within burr holes for delivering AEF therapy. As shown in the example of FIG. 22, system 2200 includes IMDs 2206 and 2208 connected to leads 114A and 114B, respectively, carrying respective electrodes. Each of IMDs 2206 and 2208 are sized and configured to be disposed within respective burr holes 2202 and 2204 within cranium 122. IMDs 2206 and 2208 may be referred to as a burr hole sized implants. IMDs 2206 and 2208 may be in wireless communication to synchronize stimulation delivery for AEF therapy. In some examples, IMDs 2206 and 2208 may be coupled via a wire to increase the possible electrode configurations when the electrode array can function as a singular array. In other examples, only one, or three or more, burr hole sized implants may be provided.

Burr hole sized implants may require the surgeon to make a small skin incision overlying the anticipated electrode placement site an using a drill bit (such as a perforator drill bit). A channel will be made within the skull that is a preset diameter (based on the drill bit). Then the surgeon creates an opening within the dura to expose the burr hole to the pial surface of the brain. Then a custom designed implant the size of the burr hole can be inserted. The distal end of the electrode can be tunneled to a connector which permits tunneling of an extension and connection to the stimulator/waveform generator within the implant. In some examples, a burr hole implant electrode may be paired with a large surface area contact in the brain, subdural location, or external location.

The configuration of the burr hole implant would require a number of electrodes to be placed to accommodate cerebral stimulation that may be dependent on the target tumor of interest. For example, the implantation strategy might include unilateral or bilateral placement of frontal, occipital, and temporal electrodes. Existing procedures such as pial synangiosis techniques in vascular neurosurgery implement numerous burr holes performed during a single surgery. However, this technique would require multiple incisions or a single very large incision. The distance between electrodes could be between 2 and 15 cm, but smaller or larger distances may also be appropriate for AEF therapy. The position of the burr hole implant(s) would be determined based on patient specific target location 1010 and other stimulating electrodes (e.g., electrodes disposed in the brain, a subdural location, or an external location).

Stimulation voltage for AEF from burr hole implants could be as large as is feasible for an implantable generator of the allowable size that would still avoid thermal injury to tissue. This is because the larger the input voltage the stronger the resulting electric field values within the target tissue to provide greater therapy efficacy. In other examples, one or more electrodes may be coupled to an IMD and placed within a respective burr hole to reach the brain. Stereotactic EEG or other sensing or stimulation configurations may be used. In some examples, one or more burr hole implant devices (e.g., IMDs 2206 and 2208) could be combined with a cranioplasty implant device (e.g., IMD 2106) to provide a non-cerebral implanted application of AEF therapy to the brain. Such configurations may be particularly useful for superficial tumors.

In some examples, bone-screw electrode implants may be used in addition to, or instead of, the other implanted electrodes described herein. For example, one or more bone-screw electrodes may be coupled to an IMD in order to improve wide range electrical fields and/or increase sensing capabilities to superficial areas of the brain. The dimensions of the bone-screw electrodes may be dependent on the cranium thickness within the target region of the patient. This minimally invasive approach to lead implantation may include either multiple incisions or a single very large incision to expose the cranium and permit distal lead connection.

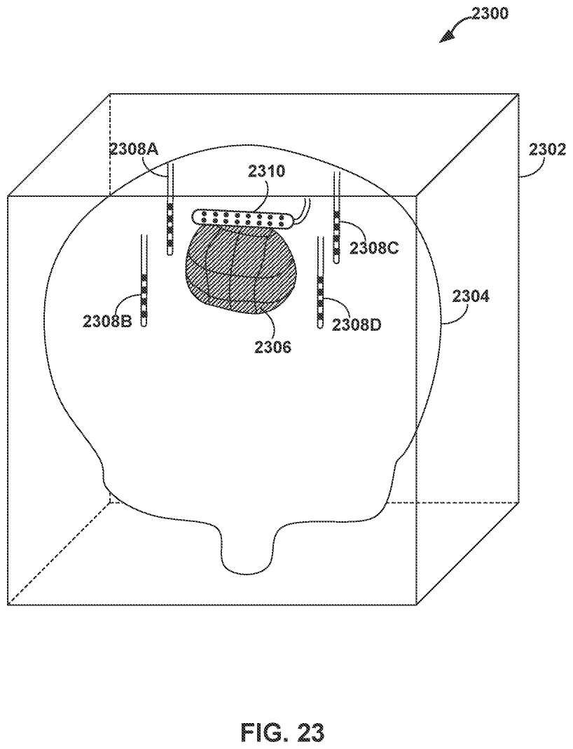

FIG. 23 is a conceptual diagram of an example three-dimensional user interface for planning implantation of electrode arrays for AEF therapy. As shown in FIG. 23, user interface 2300 may be displayed as a three-dimensional representation (on a two-dimensional screen or a three-dimensional interface) of various elements associated with AEF therapy. User interface 2300 provides three-dimensional environment 2302 that includes brain 2304, leads 2308A, 2308B, 2308C, and 2308D (collectively “leads 2308”). Resection bed 2306 indicates the location of the area from which a glioblastoma tumor was removed. Initially, imaging data may be used to identify the location, size, and shape of resection bed 2306 with respect to other tissues or structures (not shown) identified within brain 2304.

As described herein, depth electrodes (e.g., electrodes carried by leads 2308) may be placed around periphery of resection bed 2306 paired to create a “force field” surrounding resection bed 2306 thereby hindering inward progression of any cancerous cells. Planning of AEF therapy may include determining the number of electrodes, the distance from electrodes to resection bed 2306, and the distance between electrodes. In some examples, the size of electrodes may also be determined. Small electrodes typically create a more focal electrical field close to the electrodes and have trouble reaching deeper tissue. Therefore, when placed within a tumor, small electrodes may be beneficial. For implantation strategies that involve electrode placements farther away from the target tissue, larger electrodes may be advantageous. Various thermal considerations for electrode sizes and shapes may also be considered, as well as electrode materials. Certain electrode characteristics may also be selected to be MRI comparable where appropriate. In this manner, the system may be configured to provide a “tool box” of electrode options that are compatible with implantation for AEF therapy delivery and planning software. Combinations from the tool box may be more appropriate for certain lesions dependent on the target anatomy of interest. In addition to electrodes, any implanted devices or systems described herein may be configured to be compatible with MRI, computed tomography imaging, radiation therapy, or any other imaging modalities. Such compatibility may include selection of compatible materials, compatible structure shapes and/or sizes, or the ability to turn device functionality on or off as needed to reduce interference with imaging systems.

As shown in FIG. 23, leads 2308 may be disposed around the periphery of resection bed 2306 and combined with one or more paddle lead 2310 disposed a surface of resection bed 2306. Paddle lead 2310 may be any array of electrodes that can be placed near the boundary of the tumor resection. This close placement of paddle lead 2310 may reduce complexity during or after resection of the tumor and may provide a larger surface area to cover the tissue within and/or near resection bed 2306. Instead of paddle lead 2301, any electrode strips or electrode coils may be used to provide a similar coverage effect.

In any effect, it may be desirable to deliver AEF therapy near resection bed 2306 because the resection bed 2306 is generally a place for tumor regrowth. In one example, one or more balloon or expandable elements can be combined with electrodes or conductive surface within resection bed 2306 to permit appropriate positioning of the electrodes along the irregular interior of the resection bed. In one example, a balloon placement vehicle may be used to place one or more electrodes around resection bed 2306 before removing the balloon placement vehicle. In some examples, the clinician may use a bio-compatible adhesive agent (such as DuraSeal) to affix electrodes at desired locations along the surface of the target organ or resection bed 2306. Additional electrodes or leads may include 3D printable electrode structures configured to match patient-specific resection bed 2306, other tissues, or optimize therapy placement for the specific patient.

Example types of leads 2308 may include paddle leads for spreading electrical fields or cylindrical leads (as shown in FIG. 23). Electrodes with acute edges or shapes can deliver higher voltage density at those surfaces, and may include star shape electrodes, zig-zag electrodes, many curved and/or angled portions, or other shapes. In some examples, electrode shapes may be configured for patient anatomy and/or resection bed 2306 following surgical resection. In some examples, patient specific sizes of electrodes or leads may be used or modular electrode designs can be configured, assembled, or cut to size in the operating room for a specific target geometry. For example, patient specific dimensions, size of electrodes, electrode spacing, or number of electrodes can be specified for any custom array for a patient. In other examples, a patient-specific three-dimensional form of electrode or array of electrodes may be used to fit the cavity

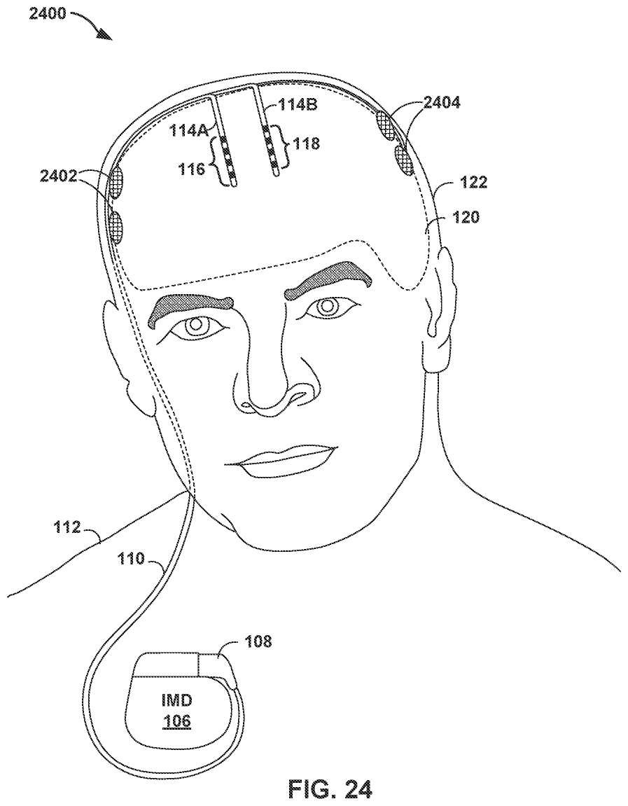

FIG. 24 is a conceptual diagram illustrating example subcutaneous electrodes for use in delivering AEF therapy. As shown in FIG. 24, system 2400 includes IMD 106 connected to leads 114A and 114B carrying respective electrodes 116, 118. In addition, subcutaneous electrodes 2402 and 2404 are controlled by IMD 106 implanted beneath the skin and external of cranium 122 of patient 112. In this manner, processing circuitry 210 of IMD 106 can be configured to control an IMD 106 to deliver a first electric field from a first electrode combination of implanted electrodes and control IMD 106 to deliver, alternating with the first electric field, a second electric field from a second electrode combination of implanted electrodes different than the first electrode combination. The first electric field and the second electric field include AEF therapy deliverable to patient 112, and at least one of the first electrode combination or the second electrode combination comprises one or more subcutaneous electrodes 2402 and/or 2404. In this manner, IMD 106 may be configured to provide wide volume electrical fields while also providing smaller electrical fields from leads 114.

In some examples, the first electrode combination or the second electrode combination comprises one or more electrodes carried by one or more implantable leads. Put another way, two or more electric fields of the AEF therapy may all include at least one electrode from leads 114. In other examples, one electric field may be provided by only some or all of subcutaneous electrodes 2402 and 2404 and another electric field may be provided by only some or all of electrodes 166, 118. In some examples, subcutaneous electrodes 2402 and 2404 may enable IMP 106 to deliver bihemispherical wide volume AEF therapy.

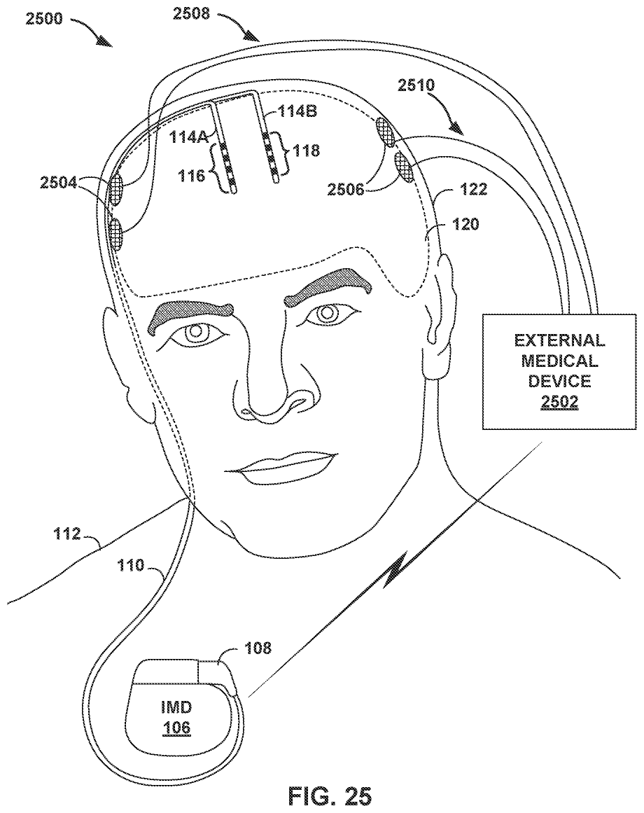

FIG. 25 is a conceptual diagram illustrating example cutaneous electrodes and external medical device for use in delivering AEF therapy. As shown in FIG. 25, system 2500 includes IMP 106 connected to leads 114A and 114B carrying respective electrodes 116, 118. In addition, external cutaneous electrodes 2504 and 2506 are controlled by external medical device 2502. In this manner, processing circuitry 210 of IMP 106 can be configured to control IMP 106 to deliver a first electric field from a first electrode combination of electrodes carried by one or more implanted leads and control external medical device 2502 to deliver, alternating with the first electric field, a second electric field from a second electrode combination of external cutaneous electrodes 2504 and 2406, where the first electric field and the second electric field comprise AEF therapy deliverable to patient 112. In other examples, external electrodes 2504 and 2406 may be directly coupled to IMD 106 such that IMD 106 controls electric fields produced by implanted and external electrodes.

In some examples, IMD 106 includes the processing circuitry configured to control external medical device 2502 to deliver the second electric field. In other examples, external medical device 2502 comprises processing circuitry configured to control IMD 106 to deliver the first electric field. In this manner, external medical device 2502 and IMD 106 may be configured to directly wirelessly communicate with each other in order to synchronize the delivery of the AEF therapy. In other example, an external programmer (e.g., programmer 104) may be configured to wirelessly communicate with IMD 106 and external medical device 2502 to coordinate delivery of the AEF therapy.

In some examples, the first electrode combination or the second electrode combination comprises one or more electrodes carried by one or more implantable leads 114. Put another way, two or more electric fields of the AEF therapy may all include at least one electrode from leads 114. In other examples, one electric field may be provided by only some or all of cutaneous electrodes 2504 and 2406 and another electric field may be provided by only some or all of electrodes 116, 118. In some examples, cutaneous electrodes 2504 and 2506 may enable IMD 106 to deliver bihemispherical wide volume AEF therapy.

External cutaneous electrodes may enable system 2500 to improve efficacy of therapy or adjust AEF therapy as a tumor evolves. For example, external electrodes may be used to deliver AEF therapy on a regular basis or to enhance therapy at select times (e.g., provide increased electrical field coverage while patient 112 is sleeping). In some examples, external electrodes may enable adjustments to new locations of target tissue without additional surgery. In some examples, system 2500 may be configured to pass current between implanted electrodes and external electrodes. System 2500 may be configured to provide electrical fields between external electrodes and then activate various electrodes 116, 118 in order to steer these electrical fields to target tissues. Although cutaneous electrodes 2504 and 2506 are described as example external electrodes, other external electrodes may be used. For example, penetrating transcutaneous needles could serve as “external” electrodes and used in these techniques.

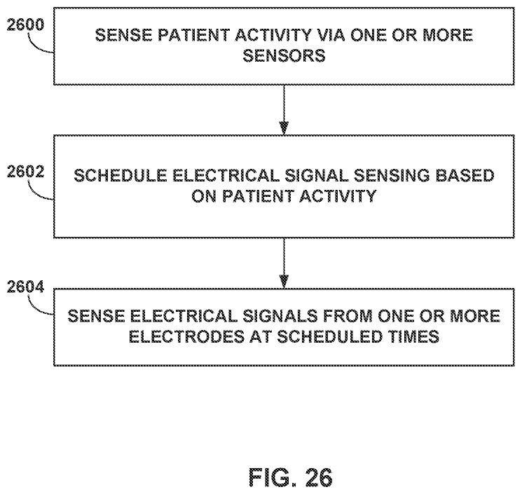

FIG. 26 is a flowchart illustrating an example technique for scheduling electrical field sensing based on patient activity. The technique of FIG. 26 will be described with respect to processing circuitry 210 of IMD 106 in FIG. 2. However, other processors, devices, or combinations thereof, such as processing circuitry 310 of programmer 104 or some combination of devices or processors (e.g., server 130), may perform the techniques of FIG. 26 in other examples.

In the example of FIG. 26, processing circuitry 210 can control one or more sensors, such as sensor 212, to sense patient activity (2600). Processing circuitry 210 then can schedule, based on the sensed patient activity, electrical signal sensing using sensing circuitry 204 (2606). Since muscle activity, cardiac cycles, breathing, or patient movement may introduce noise in electrical signals that can be sensed, processing circuitry 210 may schedule electrical signal sensing during relatively quiet period. In some examples, processing circuitry 210 may gate electrical sensing during periodic activity such as cardiac cycles or breathing. Then, processing circuitry 210 can control sensing circuitry 204 to sense electrical signals from one or more electrodes at the schedule time(s) (2604).

In this manner, a system may include an activity sensor (e.g., sensor 212) configured to generate activity data indicative of patient activity and sensing circuitry configured to sense an electrical signal through at least a portion of target tissue configured to receive alternating electric field (AEF) therapy. Processing circuitry 210 can also be configured to receive the activity data from the activity sensor, control, based on the activity data, sensing circuitry 204 to sense the electrical signal, and control, based on the electrical signal, IMD 106 to deliver AEF therapy. The sensed electrical signals may be any electrical signal or electrical field described herein that may be used as feedback to inform AEF therapy efficacy. For example, processing circuitry 210 may be configured to control sensing circuitry 204 by at least scheduling sensing circuitry 204 to sense the electrical signal during a period of reduced patient activity. Sensing circuitry 204 may be configured to sense the electrical signal by sensing an electrical field via two or more implanted electrodes.

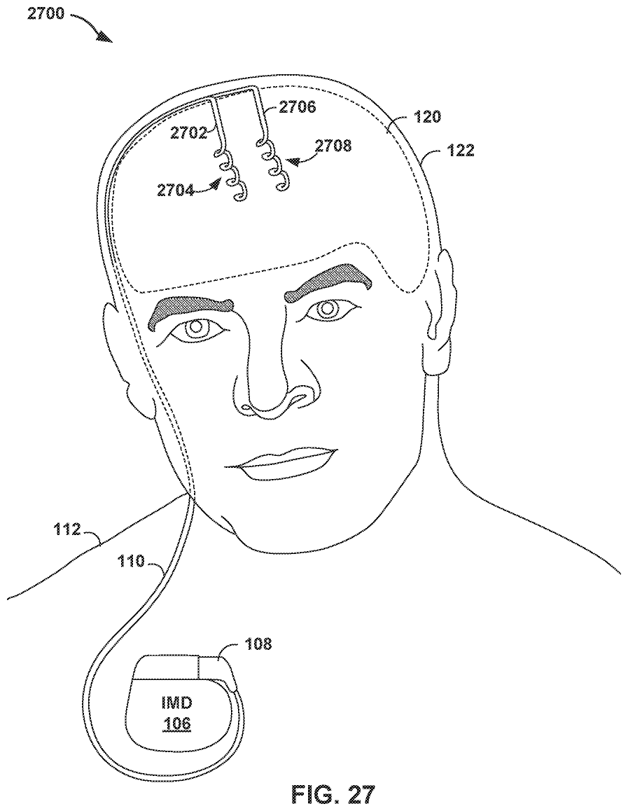

FIG. 27 is a conceptual diagram illustrating example implantable coils for delivering alternating magnetic field (AMF) therapy. As shown in the example of FIG. 27, system 2700 includes IMP 106 couped to leads 2702 and 2706 via lead extension 110. Leads 2702 and 2706 include coils 2704 and 2708, respectively. Coils 2704 and 2708 may be configured to produce a magnetic field in response to IMD 106 driving electrical current through each coil. The magnetic fields from each coil may be alternating in time in order to produce an effect similar to AEF therapy described herein in order to provide various treatments, such as reducing or preventing cell division for treating glioblastoma. The range of magnitude for delivered AMF therapy may be generally between 0.1 millitesla (mT) and 1 mT, but smaller or larger magnitudes may be used in other examples.

Although coils 2704 and 2708 are shown with large and spaced out turns, each coil may be much smaller and closely packed turns in other examples. In any example, coils 2704 and 2708 may be configured to generate respective magnetic fields may alternate in time as controlled by IMD 106. Coils 2704 and 2708 are shown positioned with the coil axes parallel to each other, but the coil axes may be orthogonal or oblique to each other or disposed in other positions. Three or more coils may be implanted in other examples. Moreover, in some examples, IMD 106 may be coupled to one or more implantable coils and two or more electrodes to produce alternating fields of an electric field and a magnetic field, respectively. The delivery of these two modalities can be temporally simultaneous or temporally interleaved dependent on which combination provides highest efficacy within the tissue type of interest. Therefore, in some examples, the system may utilize one or more feedback variables (e.g., user input and/or sensed signals) to adjust parameters that define electrical field and/or magnetic field delivery over time to titrate therapy towards clinically efficacious results.

In this manner, system 2700 can include IMD 106 which includes processing circuitry configured to receive a request to deliver AMF therapy and determine therapy parameter values that define the AMF therapy, where the AMF therapy comprises delivery of a first magnetic field and a second magnetic field. The processing circuitry can then control an IMD 106 to deliver the first magnetic field from at least implantable coil 2704 and control IMD 106 to deliver, alternating with the first magnetic field, the second magnetic field from implantable coil 2708.

Extremely low frequency electromagnetic fields (ELF-EMF) can be capable of impacting cell division, as the magnetic field lines alternating between magnetic fields may provide a similar mechanism of cell division disruption as caused by AEF therapy. Such AMF therapy may be able to inhibit any types of cell division, which may include glioblastoma cancers, breast cancer, or other types of cancer cells. Although the mechanism of AEF may be similar to AMF, AMF therapy may require different lead designs, different placements, different amplitudes, different period of time, or other changes. In some examples, AMF therapy may be used to reduce neuroplasticity. Given the defined role of transcranial magnetic stimulation (TMS) for impacting neuroplasticity, system 2700 or other similar AMF system may deliver post-debulking to enhance recovery (magnetic or electric) or reduce inflammation.

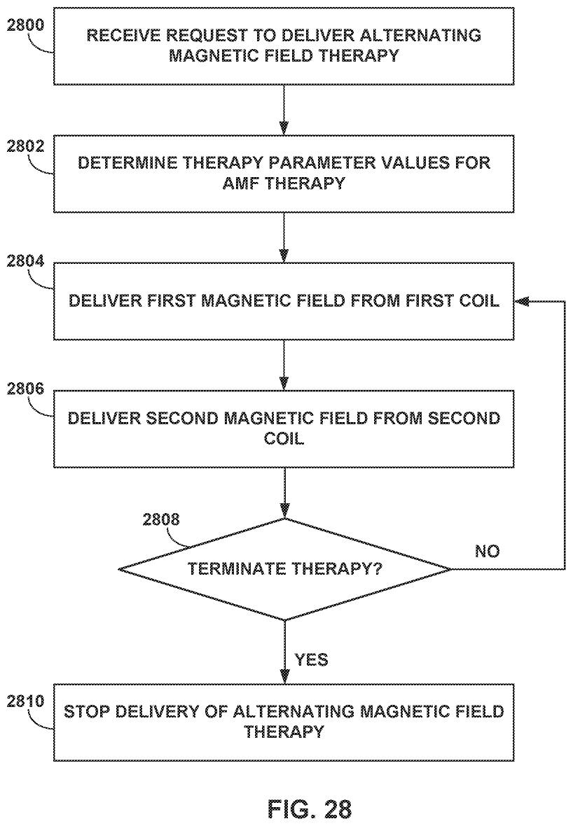

FIG. 28 is a flowchart illustrating an example technique for delivering AMF therapy using multiple implantable coils. The technique of FIG. 28 will be described with respect to processing circuitry 210 of IMD 106 in FIG. 2. However, other processors, devices, or combinations thereof, such as processing circuitry 310 of programmer 104 or some combination of devices or processors, may perform the techniques of FIG. 28 in other examples.

As shown in the example of FIG. 28, processing circuitry 210 receives a request to deliver alternating magnetic field (AMF) therapy (2800), such as from external programmer 104 or a pre-programmed delivery schedule. Processing circuitry 210 then determines therapy parameter values for AMF therapy (2802). This determination may be retrieval of parameter values from memory or determining one or more parameter values based on a delivery schedule, sensed data, or any other information.

Processing circuitry 210 then delivers the AMF therapy by delivering a first magnetic field from a first coil (2804) alternating with delivery of a second magnetic field from a second coil different than the first coil (2806). In some examples, the first and second magnetic fields may be interleaved such that they do not overlap. In other examples, the first and second magnetic fields may be partially overlapping in time. Although the first and second magnetic fields may be delivered with the same amplitude and frequency, the first and second electrical fields may be defined by different amplitudes and/or different.

Processing circuitry 210 then determines whether to terminate the AMF therapy (2808). If processing circuitry 210 determines that AMF therapy is not to be terminated (“NO” branch of block 2808), processing circuitry 210 continues to deliver the first and second magnetic fields (2804 and 2806). If processing circuitry 210 determines that AMF therapy is to be terminated or otherwise paused (“YES” branch of block 2808), processing circuitry 210 stops delivering the AMF therapy to the patient (2810).

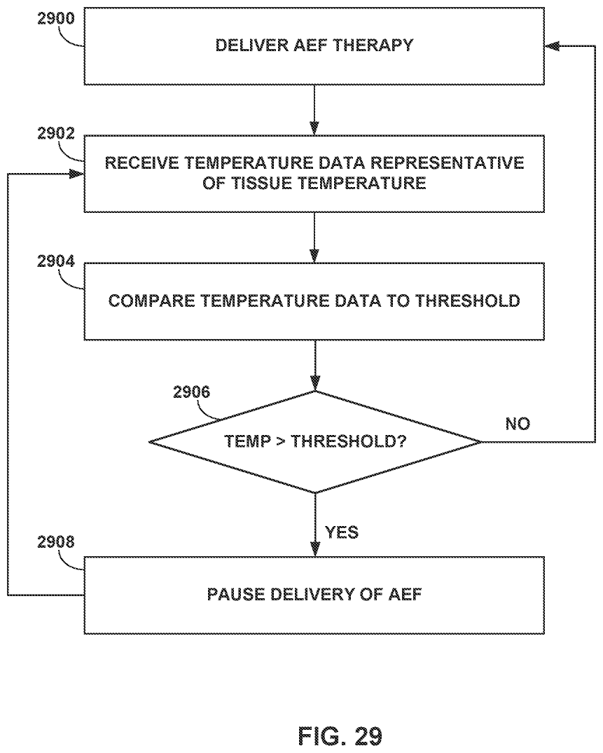

FIG. 29 is a flowchart illustrating an example technique for cycling AEF therapy based on sensed temperatures of patient tissue. The technique of FIG. 29 will be described with respect to processing circuitry 210 of IMD 106 in FIG. 2. However, other processors, devices, or combinations thereof, such as processing circuitry 310 of programmer 104 or some combination of devices or processors (e.g., server 130), may perform the techniques of FIG. 29 in other examples.

In the example of FIG. 29, processing circuitry 210 delivers AEF therapy (2900). Processing circuitry 210 then receives temperate data representative of tissue temperature (2902). In some examples, processing circuitry 210 controls a temperate sensor to generate temperature data at a predetermined schedule or on demand. In other examples, the temperature sensor continually or periodically transmits the temperature data which can be sampled or otherwise detected by processing circuitry 210. Then, processing circuitry 210 compares the temperature data to a threshold temperature (2904). This process may enable the system to monitor tissue for elevated temperatures caused by AEF therapy that could damage tissue if exceeding safe temperature levels. For example, the threshold temperature may be 43 degrees Celsius. In other examples, processing circuitry 210 may track a cumulative thermal dose that tracks a given heat for a certain period of time and compare that cumulative therapy dose to a threshold thermal dose above which may cause tissue damage.

If processing circuitry 210 determines that the temperature of tissue is not greater than the threshold temperature (“NO” branch of block 2906), processing circuitry 210 continues to deliver AEF therapy (2900). If processing circuitry 210 determines that the temperature of tissue is greater than the threshold temperature (“YES” branch of block 2906), processing circuitry 210 pauses or cancels delivery of AEF therapy (2908). Then, processing circuitry 210 may continue to monitor the temperature data and withhold AEF therapy until the temperature drops back down below the threshold temperature.

In this manner, processing circuitry 210 may be configured to receive temperature data generated by a temperature sensor, the temperature data indicative of temperature at a tissue region of a patient, and control, based on the temperature, IMD 106 to deliver alternating electric field (AEF) therapy via a plurality of implantable electrodes. In some examples, processing circuitry 210 is configured to determine, based on the temperature data, that the temperature exceeds a threshold temperature and, responsive to the temperature exceeding the threshold temperature, control IMD 106 to terminate AEF therapy. In addition, processing circuitry 210 may be configured to determine, based on the temperature data, that the temperature drops below an acceptable temperature and, responsive to the temperature dropping below the acceptable temperature, control IMD 106 to redeliver the AEF therapy.

In some examples, AEF devices may utilize high voltage (50V peak or 100 Vpp) which can cause electrodes to reach critical temperature that can damage tissue. For example, AEF device can increase tissue temperatures may several degrees in some cases. This tissue temperature may be a specific temperature, such as 41 degrees Celsius, or may be defined by a quantify of heat received over a period of time. Exceeding such temperatures may trigger IMD 106 to terminate AEF therapy to allow the tissue to cool. For example, IMD 106 may cycle AEF therapy on or off, spread out alternating electrical field switching, or otherwise reduce delivered heat to the tissue. In some examples, instead of turning off stimulation, IMD 106 may reduce the amplitude of the electrical fields which can reduce tissue heating. In any event, the smaller voltages used for implanted AEF therapy may cause less heating than external electrode based AEF therapy.

In some examples, AEF therapy may cause certain changes to tissue. For example, AEF therapy may breakdown the blood brain barrier. AEF within the cranium has been demonstrated to impact the integrity of the blood brain barrier. This has been suggested as a potential reason why some chemotherapy treatments have been suggested to be augmented with concurrent AEF treatment. In this manner, AEF therapy can be delivered as a standalone treatment or as an augmentation strategy to facilitate the efficacy of other treatment methods. In some examples, the timing of adjuvant treatments, such as chemotherapy, should be concurrent to the application of AEF to the tumoral environment. Implanted AEF therapy may provide an improvement over external systems because the patient could receive AEF therapy at any time necessary to augment the other treatment modality.

Different AEF parameter values may increase the blood brain barrier breakdown effect. For example, certain frequencies of the electrical fields may improve blood barrier breakdown. In one case, 100 kHz (or some similar frequency) may be the frequency value for blood brain barrier disruption, following a test of the range from 100 kHz-300 kHz. In some examples, the timing of chemotherapy agents or other drugs may be set based on when AEF therapy is delivered, or AEF therapy triggered according to the dosing schedule of the agents.

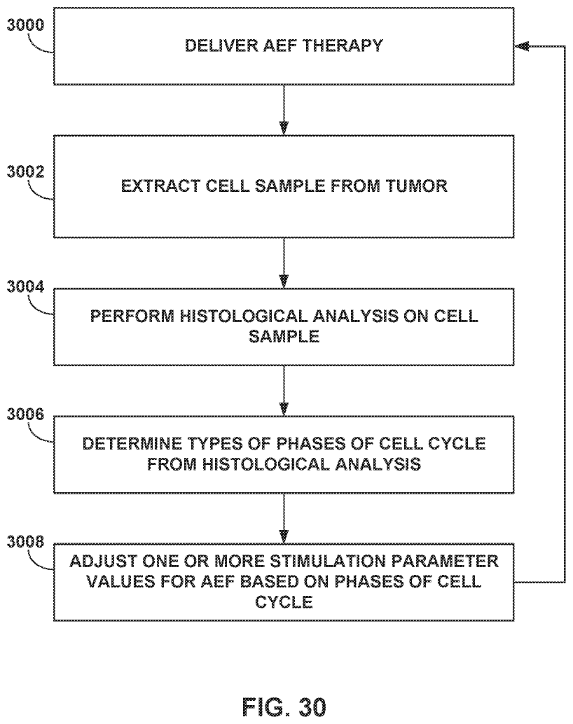

FIG. 30 is a flowchart illustrating an example technique for synchronizing AEF therapy to cell cycle phases. The technique of FIG. 30 will be described with respect to processing circuitry 310 of programmer 104 in FIG. 3. However, other processors, devices, or combinations thereof, such as processing circuitry 210 of IMD 106 or some combination of devices or processors (e.g., server 130), may perform the techniques of FIG. 30 in other examples.

In the example of FIG. 30, processing circuitry 310 controls IMD 106 to deliver AEF therapy to a target tissue (3000). A clinician later extracts a cell sample from the tumor or resection bed from the patient (3002). The clinician or other professional may perform histological analysis on the cell sample (3004). The clinician or other professional may then determine the types and duration (timing within and between) of phases of cell cycle from the histological analysis (3006). The phases of the analyzed cells may inform the clinician as to how the AEF therapy is affecting the cell cycle. The clinician or processing circuitry 310 can then adjust one or more stimulation parameter values, such as cycling time, for subsequent AEF therapy based on the phases of the cell cycle determine in the histological analysis (3008).

Given that AEF therapy has been shown to increase the number of cells that are within the G2 state of the mitotic cell cycle (and also translates to enlarged physical cell size), AEF therapy can be utilized as a synchronizer of the cell cycle within a population of cells. Synchronization of the cell cycle phase within a population of cells has numerous applications throughout practically all cell types within the body. For oncology, a clinician or system may improve chemotherapeutic or radiation therapy efficacy using AEF therapy because different chemotherapy agents can impart cellular effects within certain phases of the cycle. For non-oncological use cases, a clinician may enhance or select for the output of bone marrow or synchronize cells to increase the number of cells at a stage where a given drug is most effective. In addition, the AEF therapy may be used to synchronize cell division state at a prior stage (so the cells all transition to target stage together with the appropriately timed drug exposure). In some examples, the cells may benefit from staying in a phase, e.g., G2, longer than other phases of the cycle. In addition, vitamin benefits may be realized from AEF therapy by nutritionally synchronizing for cell division. AEF can serve as a trigger for a secondary agent at or after the optimal phase of the cell cycle, such as after the cells have been exposed to AEF to have been synchronized. In one example, the system may deliver a first regime of AEF (e.g., at a first frequency) to phase synchronize cells of a certain size. Then, the system may deliver a second regime of AEF (e.g., at the first frequency or a different frequency) to selectively impact cells in that phase (by altered size, etc.) to increase the impact of the delivered AEF therapy.

In some examples, certain treatment parameters of AEF therapy can select for certain phases of the cell cycle to become selected for, and therefore synchronized within, the cell population receiving therapy. Following the synchronization of the mitotic state of a cell population, a parameter dependent application of AEF can permit the system to control how the cells progress through the phases of the cell cycle (i.e. rate of individual phases, overall time for mitotic division, etc.).

In some examples, tumor cell histological analysis following tumor cell exposure to AEF therapy of a given frequency can permit determination of the population of cells within a given phase of the cell cycle (G1, S, G2/M) through FACS analysis, or immunofluorescent staining. In some examples, the system or clinician can distinguish G2 and M phase cell populations utilizing an M-phase marker (i.e. anti-phospho-histone H3 antibody). This would permit an assessment for the percentage of synchronization within the tumor cell population regarding the cell cycle when using AEF therapy. This would also permit analysis for increasing cell cycle synchronization following modulation of the stimulating frequency for AEF therapy.

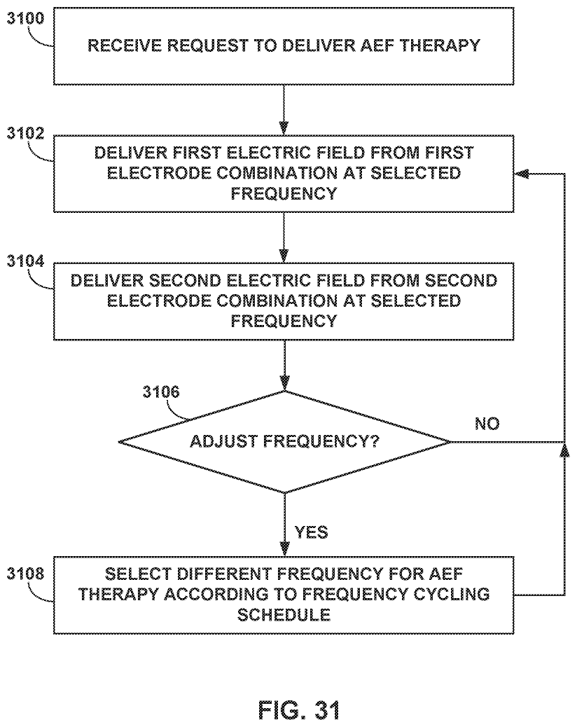

FIG. 31 is a flowchart illustrating an example technique for cycling through different frequencies for AEF therapy. The technique of FIG. 31 will be described with respect to processing circuitry 210 of IMD 106 in FIG. 2. However, other processors, devices, or combinations thereof, such as processing circuitry 310 of programmer 104 or some combination of devices or processors (e.g., server 130), may perform the techniques of FIG. 31 in other examples.

In the example of FIG. 31, processing circuitry 210 receives a request to deliver AEF therapy (3100). Processing circuitry 210 then delivers the first electric field from a first electrode combination at a selected frequency (3102) and then delivers the second electric field from a second electrode combination at the selected frequency (3104). If processing circuitry 210 determines the frequency is not to be adjusted (“NO” branch of block 3106), processing circuitry 210 continues to deliver AEF therapy at the selected frequency (3102). If processing circuitry 210 determines that adjustments should be made to the frequency as part of cycling (“YES” branch of block 3106), processing circuitry 210 selects a different frequency for AEF therapy according to the frequency cycling schedule (3108) and again delivers AEF therapy using the new selected frequency (3102). In this manner, IMD 106 may cycle through different frequencies to affect different cells that may be affected by different therapies.

As described above, IMD 106 may provide “cyclic” AEF frequency sweeping. In one example, the frequency may be alternating between 150 kHz and 200 kHz for glioblastoma cells continuously. In other examples, the frequency sweep may include additional frequencies between those values. However, other frequencies may be used in other examples.

The distribution of AEF across a membrane is different dependent upon the physical size of the cells. Through the application of AEF therapy, the system impacts cell size by inhibiting cell division and enhancing the G2 state. Therefore, a sweeping frequency range can capture both larger cells (potentially those enhanced in size by the AEF treatment and increased G2 state of the population) and smaller cells. In some examples, IMD 106 may continuously sweep from top to bottom frequency (or vice versa) repeatedly to capture the majority of cells regardless of the current cell size. In other examples, IMD 106 may deliver AEF therapy using multiple frequencies at the same time. For example, IMD 106 may interleave the frequencies, deliver simultaneous fields at different frequencies, dwell time at each frequency, or adjust the frequencies based on sensed patient data.

For a given tumor cell type there may be a target AEF frequency to generate an inhibitory response in cell growth. Therefore, a stimulation plan could include beginning treatment at the target frequency and maintaining the treatment at that frequency for the majority of the time but perform “sweeps” to lower frequencies to “pick up” those cells that would have experienced G2 state physical cell enlargement or to accommodate the tumor cells that were innately a smaller size than the majority of the other cells within the tumor. Sweeping could also be performed to the higher frequencies such that tumor cells that are innately smaller than the majority of the remaining tumor cells could experience a more optimized inhibitor field for a portion of the stimulation interval.

In some examples, stimulation parameters defining the AEF therapy could utilize phase shifting combined with frequency sweeping. Histological analysis of tumor cells can permit personalization of the “tuneable” treatment parameters such that the clinician can use waveforms with frequencies selected for that type (or sub-population within tumor based on cell marker analysis) or size of cell based on the histological analysis. Tunable treatment parameters could be adjusted at any time during the therapy delivery process.

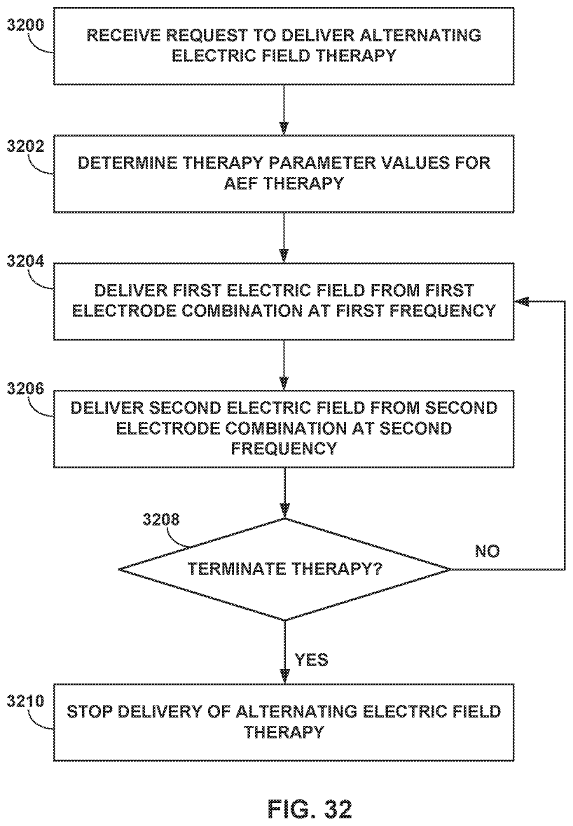

FIG. 32 is a flowchart illustrating an example technique for delivering AEF therapy that includes different frequencies from different electrode combinations. The technique of FIG. 32 will be described with respect to processing circuitry 210 of IMD 106 in FIG. 2. However, other processors, devices, or combinations thereof, such as processing circuitry 310 of programmer 104 or some combination of devices or processors, may perform the techniques of FIG. 32 in other examples.

As shown in the example of FIG. 32, processing circuitry 210 receives a request to deliver alternating electric field (AEF) therapy (3200), such as from external programmer 104 or a pre-programmed delivery schedule. Processing circuitry 210 then determines therapy parameter values for AEF therapy (3202). This determination may be retrieval of parameter values from memory or determining one or more parameter values based on a delivery schedule, sensed data, or any other information.

Processing circuitry 210 then delivers the AEF therapy by delivering a first electric field from a first electrode combination at a first frequency (3204) alternating with delivery of a second electric field from a second electrode combination different than the first electrode combination and at a second frequency different than the first frequency (3206). In some examples, the first and second electrical fields may be interleaved such that they do not overlap. In other examples, the first and second electrical fields may be partially overlapping in time. Although the first and second electrical fields may be delivered with the same amplitude and frequency, the first and second electrical fields may be defined by different amplitudes and/or different frequencies (e.g., 150 kHz and 200 kHz or other frequencies or frequency ranges). The first and second electrode combinations may use completely different electrodes or partially different electrodes, for example. The first and second electrode combinations may be selected to generate respective electrical fields that are orthogonal to each other or oblique, in some examples. Although all electrodes of the first and second electrode combinations may be implanted in some examples, one or more of the electrodes may be external electrodes in other examples.

Processing circuitry 210 then determines whether to determinate the AEF therapy (3208). If processing circuitry 210 determines that AEF therapy is not to be terminated (“NO” branch of block 3208), processing circuitry 210 continues to deliver the first and second electric fields (3204 and 3206). If processing circuitry 210 determines that AEF therapy is to be terminated or otherwise paused (“YES” branch of block 3208), processing circuitry 210 stops delivering the AEF therapy to the patient (3210). In some examples, the concept of interleaving electric fields can be utilized within the stimulation parameters, wherein different electrodes are utilized at different frequencies to permit spatially separated mixing of waveforms.

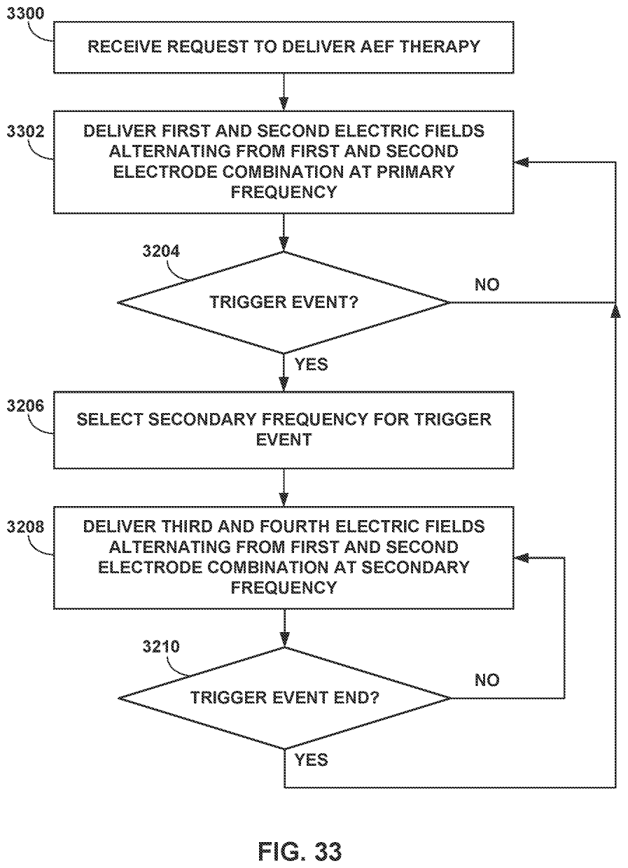

FIG. 33 is a flowchart illustrating an example technique for adjusting the frequency of AEF therapy in response to detecting a trigger event. The technique of FIG. 33 will be described with respect to processing circuitry 210 of IMD 106 in FIG. 2. However, other processors, devices, or combinations thereof, such as processing circuitry 310 of programmer 104 or some combination of devices or processors, may perform the techniques of FIG. 33 in other examples.

As shown in the example of FIG. 33, processing circuitry 210 receives a request to deliver alternating electric field (AEF) therapy (3300), such as from external programmer 104 or a pre-programmed delivery schedule. Processing circuitry 210 then delivers the first and second electric fields alternating from first and second electrode combinations at a primary frequency (3202). The primary frequency may be selected to generally treat tissue at certain cell sizes or under certain physiological conditions. If no trigger event is detected (“NO” branch of block 3204), processing circuitry 210 continues to deliver AEF therapy at the primary frequency.

If processing circuitry 210 detects a trigger event (“YES” branch of block 3204), processing circuitry selects a secondary frequency for the trigger event (3206). Example trigger events may be MRI indicated increases or decreases in cell size, tumor resection, or any other event that may change how cells react to AEF therapy. Additional exemplary trigger events include certain threshold values of electrical physics parameters as calculated by sensed electric field. Further exemplary trigger events may be a certain duration of stimulation pursued at the preceding frequency (i.e. primary or secondary). Processing circuitry 210 then delivers third and fourth electric fields alternating from first and second electrode combinations, respectively, at the secondary frequency. If the trigger event has not ended (“NO” branch of block 3210), processing circuitry 210 continues to deliver AEF therapy at the secondary frequency (3208). If processing circuitry 210 determines that the trigger event ended (“YES” branch of block 3210), processing circuitry 210 selects the primary frequency for subsequent AEF therapy once more (3202).

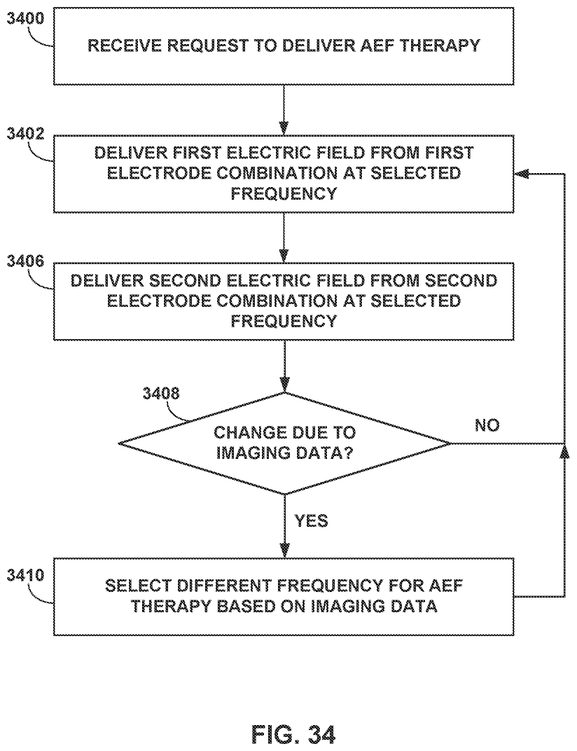

FIG. 34 is a flowchart illustrating an example technique for adjusting the frequency of AEF therapy in response to tissue changes indicated by image data. The technique of FIG. 34 will be described with respect to processing circuitry 210 of IMD 106 in FIG. 2. However, other processors, devices, or combinations thereof, such as processing circuitry 310 of programmer 104 or some combination of devices or processors, may perform the techniques of FIG. 34 in other examples.

As shown in the example of FIG. 34, processing circuitry 210 receives a request to deliver alternating electric field (AEF) therapy (3400), such as from external programmer 104 or a pre-programmed delivery schedule. Processing circuitry 210 then delivers the AEF therapy by delivering a first electric field from a first electrode combination at a selected frequency (3404) alternating with delivery of a second electric field from a second electrode combination different than the first electrode combination and at the selected frequency (3406). If processing circuitry 210 or other device or user determines that there is no change in cells from imaging data (“NO” branch of block 3408), processing circuitry 210 continues to deliver AEF therapy (3402). If processing circuitry 210 or other device or user determines that imaging data indicates a change to cells (“YES” branch of block 3408), processing circuitry 210 selects a different frequency for subsequent AEF therapy based on the imaging data (3410).

In some examples, imaging data may be obtained after a certain amount of time of AEF therapy, such as after 24 hours at a certain frequency. A change in the size of cells may be indictive of effective AEF therapy, but a change in frequency may continue to affect the cells. A broad cytokine, growth factor, and circulating biomarker panel for cell death, hypoxia, angiogenesis, inflammation, proliferation, invasion, and tumor burden can be utilized to monitor for evidence of response to treatment or tumor progression.

In some examples, ultrasound can be used to visualize tumor necrosis for the purposes of feedback on the sweeping or modification of the treatment frequency. Stem cell differentiation can be impacted by exposure to electric fields; therefore, implantable delivery of AEF can be used for inducing stem cell differentiation.

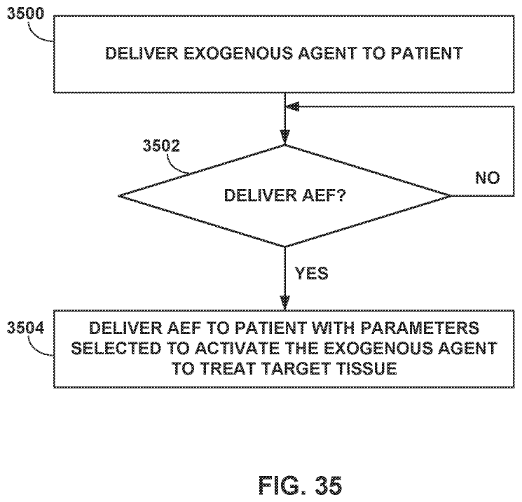

FIG. 35 is a flowchart illustrating an example technique for delivering AEF therapy to activate an exogenous agent injected into the patient. The technique of FIG. 35 will be described with respect to processing circuitry 210 of IMD 106 in FIG. 2. However, other processors, devices, or combinations thereof, such as processing circuitry 310 of programmer 104 or some combination of devices or processors, may perform the techniques of FIG. 35 in other examples.

As shown in the example of FIG. 35, a clinician can deliver an exogenous agent to the patient (3500). In other examples, an implantable pump or other device may automatically deliver the exogenous agent. Processing circuitry 210 monitors whether or not to deliver the AEF that may activate the exogenous agent (3502). If AEF is not to be delivered (“NO” branch of block 3502), processing circuitry 210 withholds AEF therapy. If processing circuitry 210 determines that AEF therapy is to be delivered (“YES” branch of block 3502), processing circuitry 210 controls IMD 106 to deliver AEF to the patient according to stimulation parameters selected to activate the exogenous agent that treats the target tissue (3504). In some examples, processing circuitry 210 controls IMD 106 to deliver AEF therapy as needed, but adjusts one or more parameters of the AEF in order to activate the exogenous agent when needed.

AEF can thus be applied to a tissue in combination with an exogenous agent (e.g., a pharmaceutical) that is modified to a more pharmacokinetically active state following exposure to the AEF. Therefore, the AEF delivered within the region of the tumor via an implant would permit selective activation of the exogenous agent. In one example, high frequency AEF can disrupt a lipid-polymer nano-/micro-particle containing a regionally significant exogenous agent such as a chemotherapy drug a chemotherapy drug. In other words, the AEF can dissolve the agent or a coating that contains the agent. Without exposure to the AEF, the lipid-polymer is inert and the nano-micro-particles do not get exposed to the body. The system can control the electrical field strength, frequency, etc., as needed to activate any substance.

AEF may generically impact the cellular transcriptomics for protein synthesis. Therefore, this is a selective impact within the tumor cells (compared to no impact in normal cells). This difference in the transcriptomic profile of a cell could serve as a method to trigger therapeutic activity of an exogenous agent.

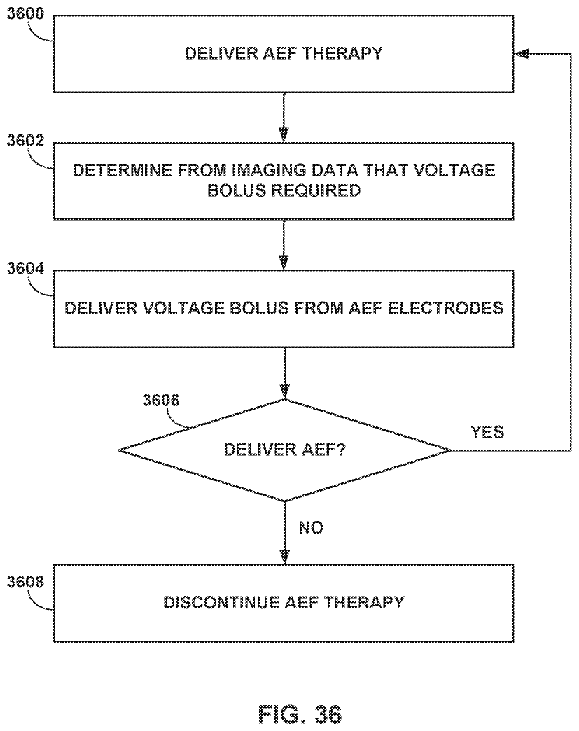

FIG. 36 is a flowchart illustrating an example technique for delivering a voltage bolus configured to cause irreversible electroporation of cell membranes in a target tissue. The technique of FIG. 36 will be described with respect to processing circuitry 210 of IMD 106 in FIG. 2. However, other processors, devices, or combinations thereof, such as processing circuitry 310 of programmer 104 or some combination of devices or processors, may perform the techniques of FIG. 36 in other examples.

AEF delivery can induce irreversible electroporation within tumor cells and thereby permit a single dose-based cell death triggering event. IMD 106 or other AEF delivery system can be utilized to give a one-time bolus of voltage to cause AEF with parameters sufficient to achieve irreversible electroporation in cells. Therefore, this may be described as a method of permissive electrode heating. In some examples, this bolus of voltage may be utilized following radiographic confirmation of tumor progression or recurrence. In other words, the electroporation may be used to halt further tumor progress and destroy existing region tumor.

As shown in the example of FIG. 36, processing circuitry 210 delivers the AEF therapy by delivering a first electric field from a first electrode combination at a selected frequency (3600). Processing circuitry 210 then determines from imaging data that a voltage bolus is required (3602). Processing circuitry 210 than controls IMD 106 to deliver the voltage bolus from the AEF electrodes sufficient to cause electroporation in the cells of the target tissue (3604). For example, the voltage bolus may create electric fields with a strength much greater than normal AEF therapy. This voltage bolus may be created by the implanted device alone, or facilitated by the addition of a temporarily applied external power source (via induction or other energy transfer means) to enhance the strength of the electric field levels.

If processing circuitry 210 determines to continue delivering AEF therapy (“YES” branch of block 3606), processing circuitry 210 continues to deliver AEF therapy (3600). If processing circuitry 210 or other device or user determines that AEF therapy is no longer required (“NO” branch of block 3606), processing circuitry 210 discontinues AEF therapy (3608).

In some examples, IMD 106 may not be configured to provide the sufficient voltage bolus with the contained power supply. Therefore, IMD 106 may utilize a transcutaneous pathway that enables IMD 106 to receive additional energy delivered from an external source. For example, IMD 106 may include a primary battery “bypass” mode that enables external energy into the implant system and through the electrodes to deliver the desired large voltage bolus. In some examples, this process may be performed under the oversight of a clinician during an office visit. In other examples, IMD 106 or another device may include the use of external cutaneous electrodes to augment the voltage bolus treatment either with additional energy delivery or field steering by selective current source and sinks using the external electrodes.

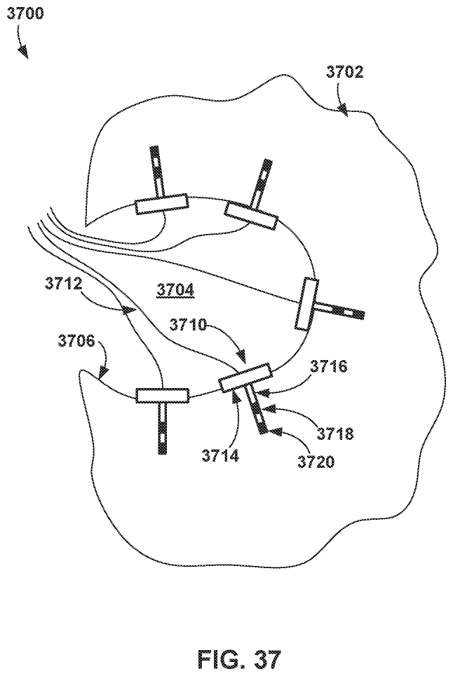

FIG. 37 is a conceptual diagram of example tack electrodes 3710 (e.g., a medical lead with one or more electrodes) configured to be implanted within the surface 3706 of a resection cavity 3704 of a patient. As shown in FIG. 37, five tack electrodes 3710 are included within lead system 3700. Lead system 3700 can be implanted within tissue 3702, which may be a portion of the brain of the patient. Surface 3706 may be the inner surface of resection cavity 3704 (e.g., a tissue resection region). Although lead system 3700 includes five tack electrodes 3710 in this example, other examples of system 3700 may include as few as one tack electrode, two or more tack electrodes, or five or more electrodes. Tack electrodes 3710 may be positioned at any position around surface 3706, which may create a planar or three-dimensional electrode array.

Each tack electrode 3710 may include components such as non-conductive cap 3714 coupled to one or more structures that may include post 3716. Post 3716 is configured to carry one or more electrodes, such as electrodes 3718 and 3720. Each of electrodes 3718 and 3720 may be disposed at different axial positions along post 3716. Different tack electrodes 3710 may be configured with different length posts 3716 and/or a different number of electrodes. The cross-sectional area of post 3716 is smaller than a cross-sectional area of non-conductive cap 3714. In this manner, post 3716 is configured to be inserted into tissue 3702 to a depth limited by a length of post 3716 extending from a distal surface of non-conductive cap 3714. In other words, non-conductive cap 3714 may be configured to be a depth stop for insertion of post 3716. Each of tack electrodes 3710 may include lead housing 3712 extending proximal from non-conductive cap 3714 in order to carry one or more conductors to IMD 106, for example. The proximal ends of lead housing 3712 can then be coupled to IMD 106. In some examples, the non-conductive cap may carry one or more electrodes in addition to, or instead of, electrodes 3718 and 3720. For example, non-conductive cap 3714 may include one or more electrodes on the surface configured to contact surface 3706 of the tissue defining resection cavity 3704.

In some examples, lead system 3700 may facilitate implantation of a large variety of numbers of tack electrodes 3710, such as only one tack electrode 3710, or a large number of tack electrodes distributed around surface 3706 of resection cavity 3704. In this manner, the clinician may select the number of tack electrodes 3710 needed to achieve appropriate coverage for electric field therapy, such as AEF therapy or other therapy, or even mix and match different types of tack electrodes 3710 (e.g., different lengths of post 3716, different number of electrodes 3718 and 3720, etc.).

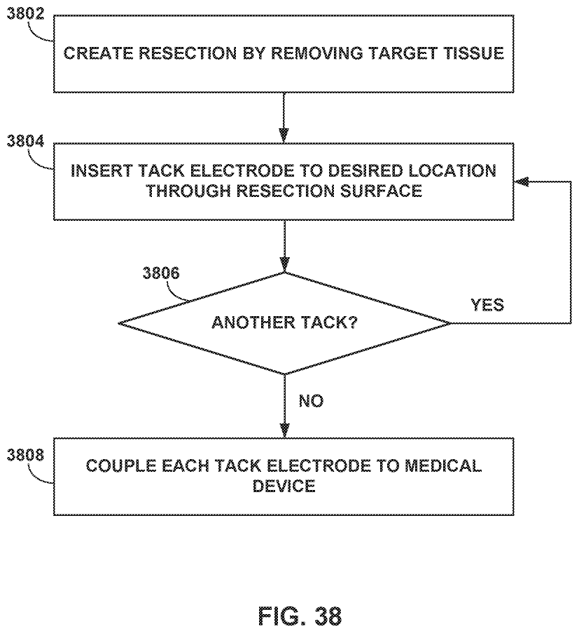

FIG. 38 is a flowchart illustrating an example technique for implanting multiple tack electrodes 3710 within a resection cavity 3704 of a patient. The method of FIG. 38 will be described with respect to lead system 3700 of FIG. 37, but the technique may be used to implant other types of individual electrodes or electrode structures.

As shown in the example of FIG. 38, a clinician creates a resection by removing target tissue (3802). The removed tissue may be identified as including a tumor or other undesirable tissue. Then, the clinician can insert one tack electrode 3710 through the surface 3706 of tissue 3702 around resection cavity 3704 at the desired location (3804). Each tack electrode may be left floating in tissue or secured using mechanical structures such as tines, surgical-like scaffolds, glues or adhesives, etc. The position of tack electrode 3710 may be chosen as part of a larger spatial electrical field target using multiple tack electrodes 3710. If another tack electrode 3710 needs to be implanted (“YES” branch of block 3806), the clinician inserts another tack electrode at the desired location (3804). If no further tack electrodes are needed to be implanted (“NO” branch of block 3806), the clinician then couples the proximal end of lead housings 3712 of each tack electrode 3710 to a medical device such as IMD 106 (3808). In this manner, IMD 106 may be configured to generate electrical field therapy or modulation, such as AEF, to the target tissue resection region using some or all of the electrodes of the implanted tack electrodes 3710. As described herein, preoperative planning may be used to determine locations to implant tack electrodes 3710, interoperative guidance to adjust tack electrode locations, and/or postoperative assessment to determine stimulation parameters that define electric field therapy to be delivered via implanted tack electrodes 3710. Tack electrodes 3710 are merely one example type of lead or electrode that can be implanted to deliver electric field therapy as described herein.

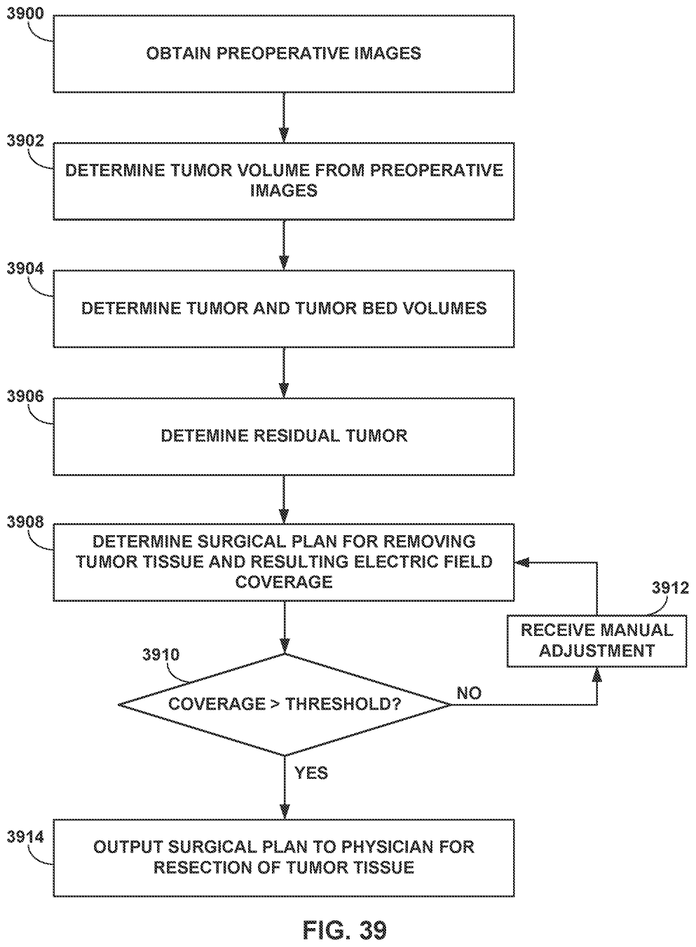

FIGS. 39, 40, and 41 may be combined to provide a full process from preoperative planning, interoperative feedback, and postoperative analysis and therapy parameter determination. FIG. 39 is a flowchart illustrating an example technique for planning tumor removal and electric field therapy. The technique of FIG. 39 will be described with respect to processing circuitry 310 of programmer 104 in FIG. 3. However, other processors, devices, or combinations thereof, such as processing circuitry 210 of IMD 106 or some combination of devices or processors (e.g., server 130), may perform the techniques of FIG. 39 in other examples.

In the example of FIG. 39, processing circuitry 310 obtains preoperative images of the patient anatomy from one or more imaging modalities (3900). Example imaging modalities include MRI, CT, x-ray, ultrasound, or other techniques for identifying tissue within the patient prior to surgery. From the preoperative images, processing circuitry 310 may determine tumor volume (3902). Processing circuitry 310 may also determine tumor shape, location, etc. from the preoperative images. In some examples, processing circuitry 310 may employ machine learning techniques to automatically identify edges of tumors from preoperative images. The machine learning model may be generated using training data from sample patients with verified tumor tissue based on feedback from surgeons or other trained professionals. Next, processing circuitry 310 can determine tumor and tumor bed volumes from the preoperative images (3904). In some examples, processing circuitry 310 may also determine any residual tumor that will remain after resection (3906). In some examples, the clinician may desire to leave some residual tumor for treatment and/or due to anatomical sensitives to removal. In some examples, processing circuitry 310 may present an augmented reality environment in which actual preoperative images are provided together with metrics or boundaries that are automatically determined or obtained via user input. Although manual surgery is described herein, it is contemplated that fully automated planning and surgery performed by robotic systems can perform the implantation, or some combination of robotic and manual surgery, can be employed to achieve the implantation of electrodes as described for electric field therapy.

Using this information regarding the anatomy of the patient, processing circuitry 310 can determine the surgical plan for removing tumor tissue and the resulting electric field coverage from the implantation of electrodes after resection (3908). In some examples, the surgical plan may encompass strategies such as certain tissue avoidance or aspects to reduce complications, such as determining a resolution of the cortical surface in order to reduce potential bleeding during surgery. Next, processing circuitry 310 may analyze the surgical plan and compare the estimated electrical field coverage to one or more thresholds indicative of appropriate therapy (3910). In response to determining that the coverage is not greater than the one or more thresholds (“NO” branch of block 3910), processing circuitry 310 can present an indication to the user and receive user adjustment (e.g., manual adjustment) to one or more aspects of the surgical plan (3912) before determining the update surgical plan (3908). If processing circuitry 310 determines that the coverage is greater than the one or more thresholds (“YES” branch of block 3910), processing circuitry 310 can output the surgical plan to the physician for resection of tumor tissue and implantation of one or more leads and electrodes for electrical field therapy (e.g., AEF therapy).

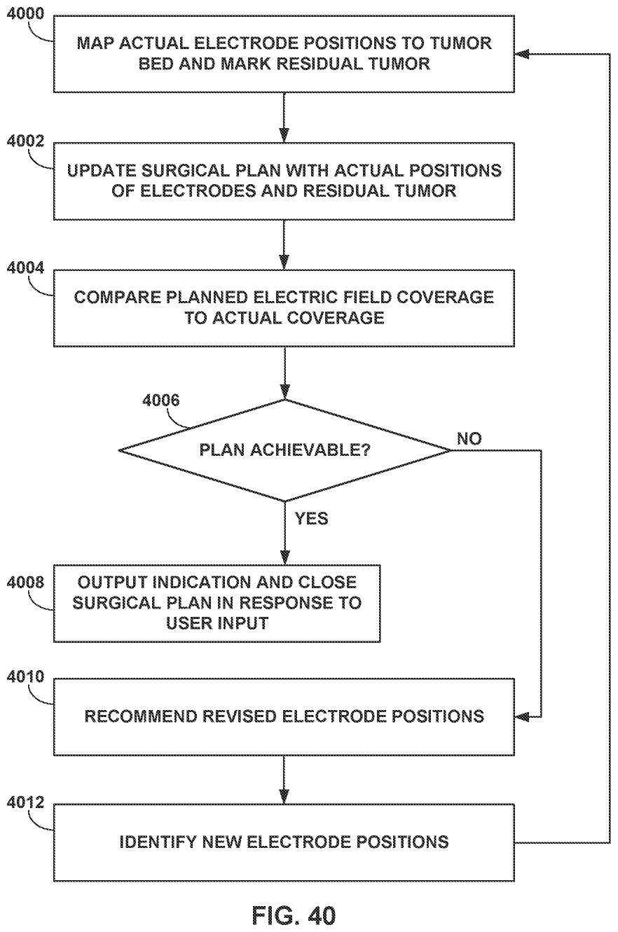

FIG. 40 is a flowchart illustrating an example technique for evaluating tumor removal and modifying electric field therapy. The technique of FIG. 40 will be described with respect to processing circuitry 310 of programmer 104 in FIG. 3. However, other processors, devices, or combinations thereof, such as processing circuitry 210 of IMD 106 or some combination of devices or processors (e.g., server 130), may perform the techniques of FIG. 40 in other examples.

In the example of FIG. 40, processing circuitry 310 can map the actual electrode positions (locations) to the tumor bed and mark the residual tumor (if any) (4000). The system may use interoperative visualization, imaging (e.g., radiographic imaging for electrode layout), registration markers, and/or user input to obtain the electrode positions. Next, processing circuitry 310 updates the surgical plan with the actual positions of the electrodes and the residual tumor (4002). As a part of this electrode location determination, processing circuitry 310 can calculate the coverage, or effective treatment area, of the electric field therapy deliverable by the implanted electrodes. Processing circuitry 310 can then compare the planned electric field coverage to actual coverage from the electrodes in their implanted locations (4004). If processing circuitry 310 determines that the surgical plan is achievable with the implanted electrodes (“YES” branch of block 4006), processing circuitry 310 outputs the indication that the plan is achievable and closes the surgical plan in response to user input to close the surgical guidance (4008).

If processing circuitry 310 determines that the surgical plan is not achievable with the implanted electrodes (“NO” branch of block 4006), processing circuitry 310 determines and recommends revised electrode positions, which may include moving implanted electrodes and/or adding additional electrodes to the already implanted electrodes (4010). The processing circuitry 310 can then identify the new electrode positions and display to the user (4012) before again mapping the actual implanted electrodes as adjusted (4000).

In some examples, robotic systems may be employed to implant the electrodes or leads according to the surgical plan. For example, the robotic system may precisely implant needle electrodes or other electrodes as described herein according to the preoperative planning of electric fields. In some examples, the robotic system may sense electric fields generated by each electrode as it is implanted and either adjust the location of that electrode or model the electric field to adjust the locations of one or more electrodes to be implanted.

In some examples, the surgical procedure may include a step of injecting cerebral fiducial markers (e.g., radiographic markers, wet clips left in the brain, dye injection, ultrasonic ablation foci, puncture holes, etc.) through craniostomies. After the craniotomy and periodically during therapy, the surgeon may use these fiducial markers to re-register the MRI images to the current cerebral position within the patient. In some examples, a CT scan or ultrasound can verify the position of the markers and re-register the scan. In this manner, the system and physician can track any changes to brain tissue during the surgery, after surgery, or over time.

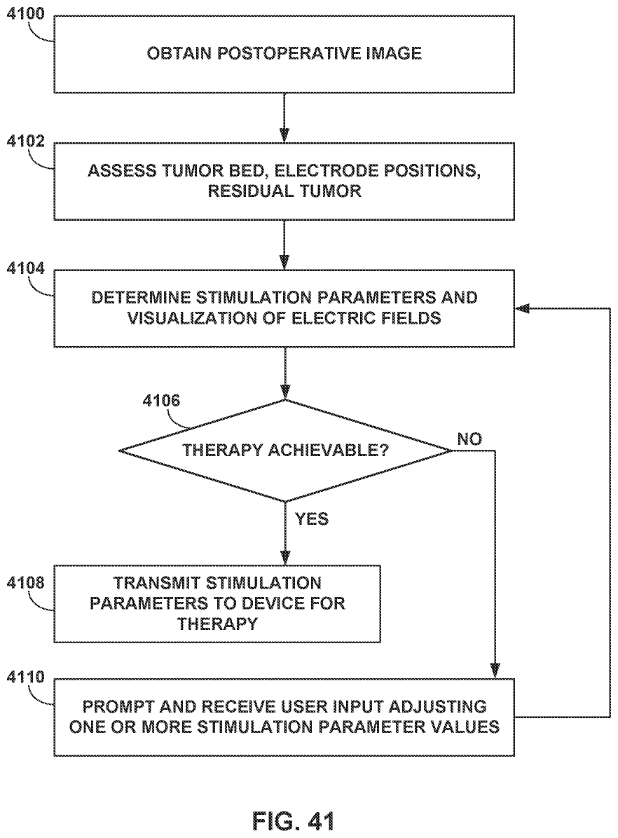

FIG. 41 is a flowchart illustrating an example technique for determining stimulation parameter values that define electric field therapy. The technique of FIG. 41 will be described with respect to processing circuitry 310 of programmer 104 in FIG. 3. However, other processors, devices, or combinations thereof, such as processing circuitry 210 of IMD 106 or some combination of devices or processors (e.g., server 130), may perform the techniques of FIG. 41 in other examples.

As shown in FIG. 41, the system may determine parameters based on the implanted system as performed in FIG. 40, for example. Processing circuitry 310 obtains postoperative images of the patient anatomy from one or more imaging modalities (4100). Example imaging modalities include MRI, CT, x-ray, ultrasound, or other techniques for identifying tissue within the patient prior to surgery. From the postoperative images, processing circuitry 310 may assess various characteristics of the remaining tissue and implanted leads, such as the tumor bed shape and volume, electrode positions, and any residual tumor location and volume (4102). Based on this assessment, processing circuitry 310 can determine stimulation parameter values that define subsequent electric field therapy and, in some examples, visualization of electric fields to be produced in the patient (e.g., based on modeling of the electric fields (4104).

Based on the determined electric fields, processing circuitry 310 can determine if the originally planned therapy can be achieved by the stimulation parameters and implanted electrodes (4106). If the planned therapy cannot be achieved (“NO” branch of block 4106), processing circuitry 310 can prompt the user for input to adjust the therapy and receive user input adjusting one or more stimulation parameter values that define electric field therapy (4110). Processing circuitry 310 can then again determine the stimulation parameters and determine if therapy is achievable. If the planned therapy can be achieved (“YES” branch of block 4106), processing circuitry 310 can transmit the stimulation parameter values to the device (e.g., IMD 106) for delivery of the electric field therapy (4108). IMD 106 (or another device) can then deliver the electric field therapy to the patient according to the stimulation parameters determined in the technique of FIG. 41.

Although the technique of FIG. 41 may be performed immediately after surgery, the technique may be revisited at any time post implant. For example, the system may be able to re-image the location of electrodes within the patient and compare the current location of electrodes to the previous location. The system can then track or identify any changes to the electrode locations and/or calculate new coverage of electric field strength given the current electrode locations. The system can, in response to any change, adjust stimulation parameter values for therapy and/or recommend adjustments to implanted electrodes. In some examples, the system may track electrode position to identify any changes to the tumor bed such as identifying new tumors or growths. The system may display these changes via representations of the anatomical changes and/or metrics indicative of the change. In response to any change exceeding a threshold, the system may recommend another surgical intervention, or the clinician may track these changes manually over time.

In some examples, processing circuitry 310 may implement any power saving techniques to reduce the amount of time the patient needs to recharge their therapy device (e.g., IMD 106). Processing circuitry 310 may generate different therapy modes, such as two or more different energy consumption modes that can be automatically selected by the system in response to one or more triggers, such as current battery charge state, therapy status, etc. The different energy consumption modes might reduce signal amplitude or increase the “off” duration (or decrease the “on” duration) for therapy (e.g., duty cycles with on/off periods on the order of seconds or minutes). In some examples, processing circuitry 310 may automatically reduce power or select a reduce power mode in response to patient activity (e.g., metabolic status, circadian rhythm, or patient availability schedule) or patient location from global positioning system (GPS) data or other location data indicative of the patient not being home to recharge the therapy device. Processing circuitry 310 may select a reduced power mode in response to battery power below a threshold in order to maintain some electric field therapy until recharging can occur to reduce lengthy amounts of time with no therapy at all.

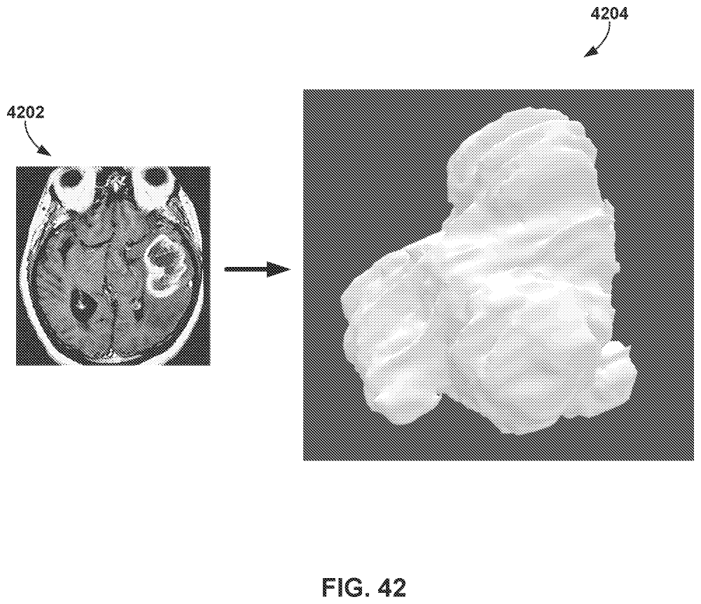

FIG. 42 is a conceptual diagram of a preoperative image 4202 and estimated volume of a tumor 4204 from the image. The system may obtain the preoperative image 4202 of the tumor and/or surrounding tissue in order to estimate the tumor 4204 volume, location, shape, or any other metric. The imaging may be obtained via MRI, CT, ultrasound, or any other modality or combination of modalities. For MRI, the system may perform enhancing volume assessment with optional inclusion or isolation of non-enhancing tumor volume assessment. In this manner, the system can isolate and analyze volume(s) of tumor tissue for implantation planning. In some examples, the system may generate metrics useful for planning a surgery or electric field (e.g., tumor treating fields) intervention. The metrics may include examples such as volume or surface area of a tumor, predicted volume or surface area of a tumor bed, suitability for electric field therapy based on maximum capability of the stimulation generator and/or expected tumor bed geometry, a number of stimulation generators and/or electrodes needed for determined characteristics of the tumor bed without removal of the tumor or after tumor removal.

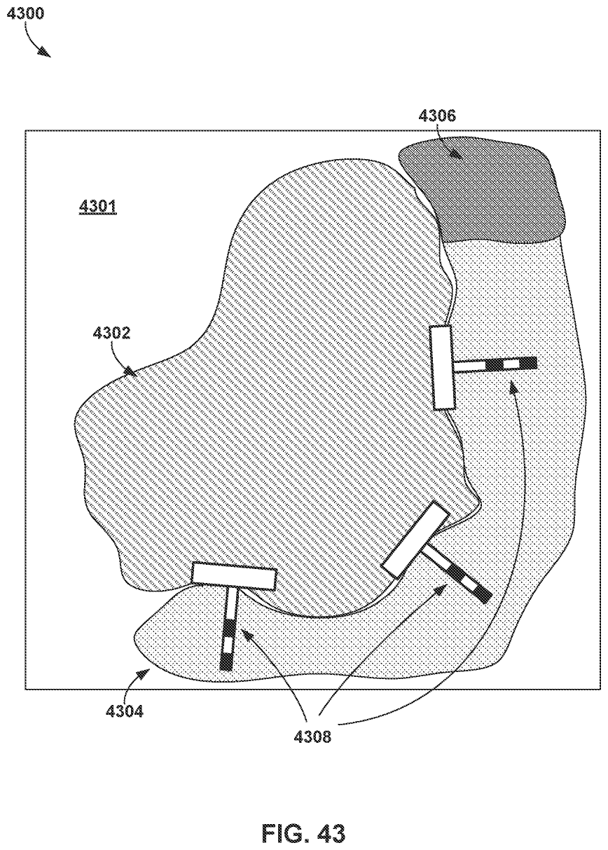

FIG. 43 is a conceptual diagram of a preoperative tumor volume determination in user interface 4300. Before the surgery, a surgeon may identify the target resection 4302 of brain tissue 4301 by providing input via user interface 4300 (e.g., user interface 4300 receives user input identifying the target resection) and based on preoperative imaging, or the system may automatically identify the target resection 4302 based on one or more tissue characteristics obtained from the image of the tissue and/or other modalities. For example, user interface 4300 may display a representation of tumor 4204 from which the system can automatically identify the tumor or the system can receive user input tracing the outline of the tumor or otherwise identifying one or more boundaries of the tumor to achieve target resection 4302. Based on the volume and/or shape of target resection 4302 the system may determine region 4304 which is a continuous region of tissue at risk for tumor recurrence. Based on the volume and/or shape of target resection 4302 and/or region 4304, the system may recommend the location and/or depth of electrodes of medical leads 4308 (e.g., tack electrodes 3710).

The system may also determine the estimated coverage over electric fields from the recommended locations of the electrodes. The system may determine this coverage by modeling the projection of electric field over the expected tissue. In some examples, the estimated coverage may be selected from a plurality of different coverage types. For example, the coverage may be set to 100% of tissue covered with a minimum threshold field strength, the coverage may be set to 80% of tissue covered with a moderate threshold field strength, of the coverage may be set to 60% of tissue covered with a high threshold field strength. These are merely examples, but indicate that the system can use different metrics for determining what it means for tissue to be covered by the electric fields. In some examples, the system may receive user input selecting the coverage type and/or the threshold field strength used by the system to determine the coverage by the electric fields.

Based on the determined coverage, the system can determine any regions of less than target coverage, such as reduced coverage region 4306. Based on the “missing coverage” over region 4306, the system may receive user input adjusting any aspects of the implantation plan, such as the number and/or location of medical leads 4308. In some examples, user interface 4300 may receive user input adding or moving medical leads 4308 or individual electrodes and re-calculate the coverage based on the updated location and/or number of electrodes.

Although user interface 4300 is shown as a two-dimensional cross section, user interface 4300 may receive user input that moves to different two-dimensional cross sectional areas. In other examples, user interface 4300 may provide a three-dimensional visualization that can be moved, zoomed in or out, and/or rotated as desired.

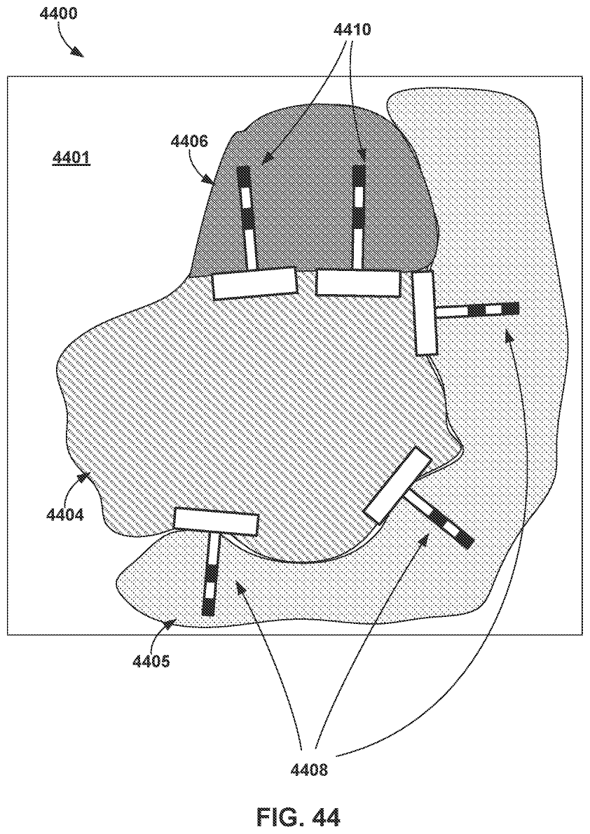

FIG. 44 is a conceptual diagram of a preoperative tumor resection plan and subsequent implantation therapy via user interface 4400. User interface 4400 may be similar to user interface 4300 of FIG. 43, but user interface 4400 indicates that the surgeon has planned to leave a residual tumor within brain tissue 4401 after resection. Before the surgery, a surgeon may identify the target resection 4404 by providing input via user interface 4400 (e.g., user interface 4400 receives user input identifying the target resection) and based on preoperative imaging, or the system may automatically identify the target resection 4404 based on one or more tissue characteristics obtained from the image of the tissue and/or other modalities. In addition, user interface 4400 may receive physician input identifying residual tumor 4406 that will remain after surgery. Based on the volume and/or shape of target resection 4405 and/or residual tumor 4406, the system may determine region 4404 which is a continuous region of tissue at risk for tumor recurrence. Based on the volume and/or shape of target resection 4404 and/or region 4405, the system may recommend the location and/or depth of electrodes of medical leads 4408 and 4410 (e.g., tack electrodes 3710). In some examples, medical leads 4410 may have longer or more electrodes than medical leads 4408 due to higher risk of the residual tumor for growth. In some examples, the leads (or burr hole caps through which the leads are inserted), may include depth adjustment mechanisms that can be physically adjusted to select the desired depth of the electrodes on the lead.

In some examples, the higher risk of residual tumor 4406 can be used for algorithmic enhancement of lead positioning within the tissue given highest priority given to residual tumor 4406 and then to contiguous region 4405 within the brain. The system may recommend a higher concentration of electrodes, stronger fields, different orientation, or other therapy characteristics for medical leads 4410 to treat residual tumor 4406 compared to medical leads 4408 of region 4405. In some examples, the system may employ duty cycle and/or power usage metrics to determine the number of electrodes and/or placement of electrodes in order to achieve the coverage needed for different region 4405 and residual tumor 4406. In some example, these power usage metrics and available coverage may be used to select a therapeutic coverage that has a lower power consumption which can reduce the frequency and/or duration of IMD 106 recharging for the patient.

The system may also determine the estimated coverage over electric fields from the recommended locations of the electrodes. The system may determine this coverage by modeling the projection of electric field over the expected tissue. In some examples, the estimated coverage may be selected from a plurality of different coverage types. For example, the coverage may be set to 100% of tissue covered with a minimum threshold field strength, the coverage may be set to 80% of tissue covered with a moderate threshold field strength, of the coverage may be set to 60% of tissue covered with a high threshold field strength. These are merely examples, but indicate that the system can use different metrics for determining what it means for tissue to be covered by the electric fields. In some examples, the system may receive user input selecting the coverage type and/or the threshold field strength used by the system to determine the coverage by the electric fields.

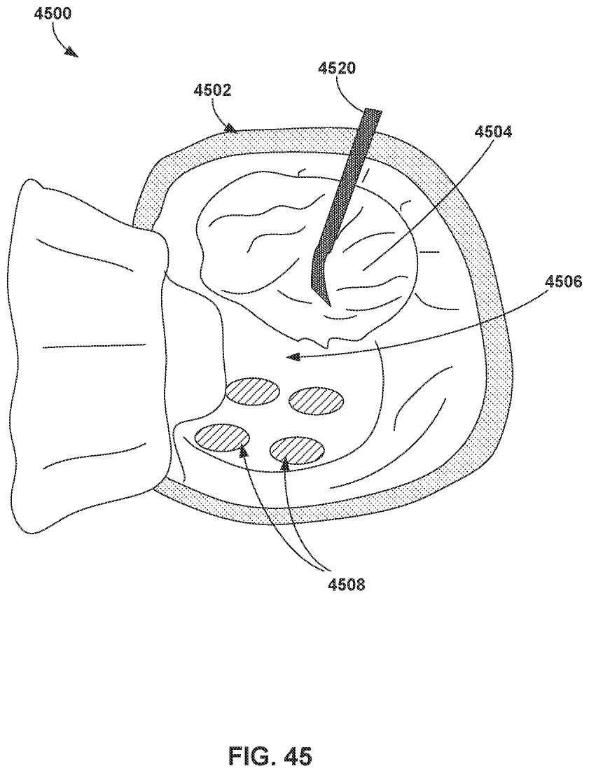

FIG. 45 is a conceptual diagram of example medical leads 4508 implanted after resection of a tumor. As shown in the example of FIG. 45, system 4500 may include medical leads 4508 implanted within the resection bed 4506 at the location where the tumor was removed. Resection bed 4506 may be located under brain portion 4504 (pulled back by tool 4520) and within cranium 4502.

In some examples, the system may provide various features to assist the surgeon in positioning the medical leads 4508 and corresponding electrodes. For example, during surgery, the system may provide one or more displays configured to present information. In one example, the display may present an augmented reality and/or virtual reality projection based on the determined visualization of the implanted medical leads 4508. In some examples, the system may display the imaging data of the tissue in a virtual space. The system may utilize photogrammetry to localize the electrodes of leads 4508 from multiple images and/or video obtained during the operation. In some examples, stereotactic placement of electrodes would allow for virtual space representation of the lead position of each of leads 4508.

In some examples, the system may present metrics regarding the placement of leads 4508 in comparison with the surgical plan. For example, the system may control the display to present visual or other indications of actual locations of leads 4508 vs. the leads in the surgical plan. Metrics presented for this difference may include a percent error, absolute measurement difference, an overall of target lead locations to the actual locations of leads 4508, and/or highlight any difference in the tumor bed or residual tumor compared with the planned procedure.

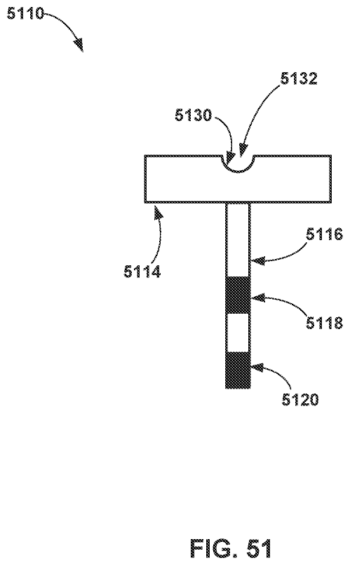

In some examples, the system may employ a semi-stereotactic placement of leads 4508 based on obtained locations from a marker on each of leads 4508 (such as registration feature 5132 of medical lead 5110 described in FIG. 51 below). In other examples, each lead may include one or more radiopaque markers or other indications observable visually or via imaging modality in order to determine the location of each electrode carried by the leads. After leads 4508 are implanted, or iteratively after each lead is implanted, the clinician may touch each registration feature of each lead with a probe connected to the system in order for the system to register each load in virtual space. In this manner, the system may map the location of every implanted lead 4508. In some examples, this mapping may also display the depth of the electrodes of each lead 4508 so that the clinician can adjust the depth of one or more leads as needed in order to establish the appropriate depth of each lead 4508.

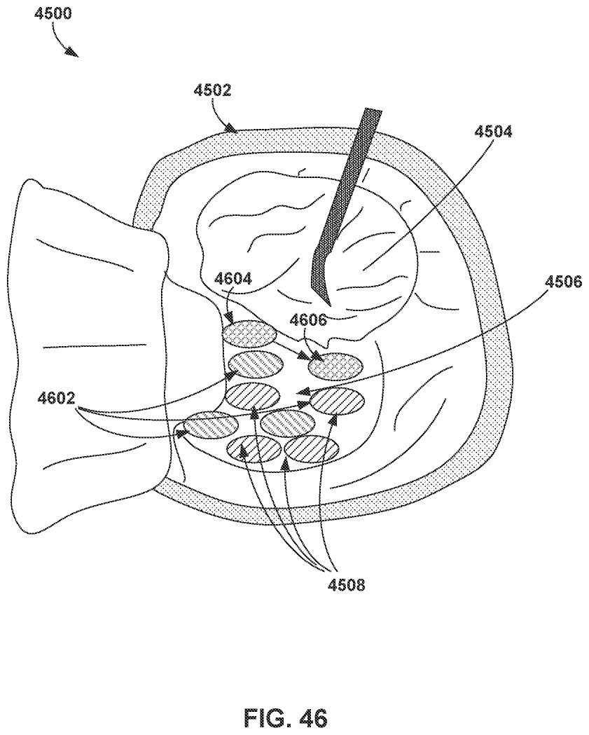

FIG. 46 is a conceptual diagram of example medical leads implanted due to a revised implantation plan. In some examples, the system can adjust they surgical plan based on the location of implanted leads 4508 compared with differences in tumor resection or any other changes that occur during surgery. The system can identify when the surgical plan needs to be altered. For example, the tumor bed may be different in volume and/or shape that requires a different array of electrodes and/or electrode movement may occur after implantation. In response to determining this change (e.g., based on user input and/or image analysis during surgery), the system can suggest additional lead placements to achieve effective therapy. For example, the system may recommend adding electrodes 4602 to tumor bed 4502. In addition, or alternatively, the system may determine that electrode 4604 should be moved to the location of electrode 4606. In this manner, the system may be configured to present alternative surgical recommendations intraoperatively in response to measured or detected deviations from the preoperative surgical plan.

In some examples, the system may employ an optional resection bed mapping step. For example, a stealth wand is used to trace the resection bed such that the system receives location data from the stealth wand and thereby draw in virtual space the limits of the resection cavity to (1) permit algorithmic calculation of the resection bed or (2) surgeon manual placement of planned leads in virtual space. In response to this intraoperative tissue description, the system can confirm and/or adjust the preoperative surgical plan. In other examples, the system may employ this interoperative mapping instead of preoperative mapping such that the system generates the lead placement recommendations based on actual tumor size and shape and/or resection bed on the fly during surgery.

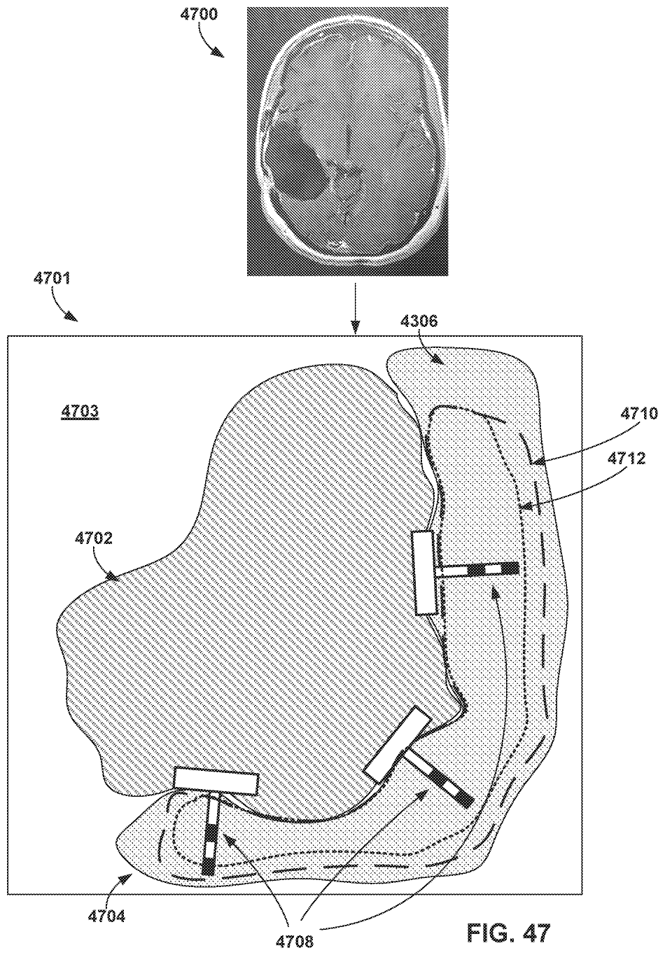

FIG. 47 is a conceptual diagram of example postoperative imaging and estimated electric field coverage from implanted leads 4708. The system may obtain postoperative image 4700 and calculate resection region 4702 (e.g., a void) within brain tissue 4703, contiguous region 4704, and implanted medical leads 4708 in user interface 4701. User interface 4701 may be similar to user interface 4300 during preoperative analysis, but updated based on the actual results of the operation.

In the example of FIG. 47, leads 4708 have been implanted in a balanced therapy plan that does not include preferential treatment for any certain tissue region. However, if there is a tissue of increased risk at tumor growth, such as a residual tumor region, leads 4708 may be concentrated in those areas compared to other tissue areas.

The system may calculate one or more coverage areas that will be possible due to electric field therapy deliverable by medical leads 4708. For example, coverage area 4712 may be indicative of the system predicted coverage of electric fields using a low performance level (e.g., low amplitude and/or longer off time during cycling) that could increase battery longevity. Coverage area 4710 may be indicative of the system predicted coverage of electric fields using a high performance level (e.g., high amplitude and/or shorter off time during cycling) that could increase the volume of treated therapy with a decrease to battery longevity. Although two coverages 4710 (dashed line) and 4712 (dotted line) are shown, only a single coverage may be displayed in some examples whereas three or more coverage options may be displayed in other examples.

In some examples, the system may employ metrics such as energy use, time between battery charges, coverage of tumor bed of region 4704 (e.g., volume of tissue), or any other metrics at various thresholds of coverage. These metrics may be displayed in association with the respective coverage in some examples.

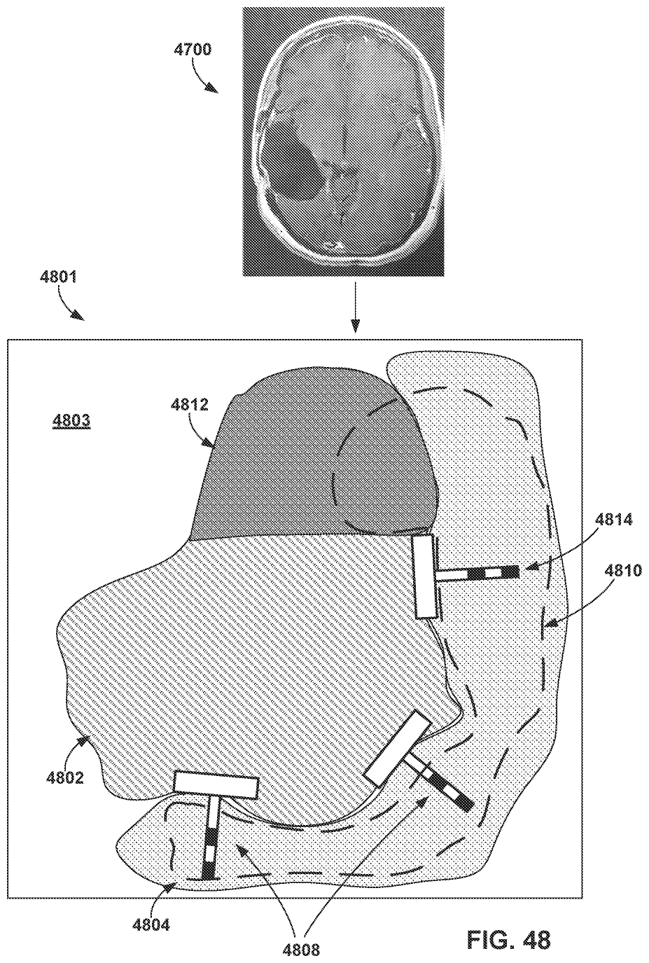

FIG. 48 is a conceptual diagram of example postoperative imaging and estimated electric field coverage from implanted leads including residual tumor designation. Similar to FIG. 47, the system can obtain postoperative image 4700 and calculate resection region 4802 (e.g., a void) in brain tissue 4803, contiguous region 4804, and implanted medical leads 4808 in user interface 4801. User interface 4801 may be similar to user interface 4701, but the clinician has designated a residual tumor that is present after surgery. User interface 4801 may be similar to user interface 4400 during preoperative analysis, but updated based on the actual results of the operation.

In the example of FIG. 48, leads 4708 have been implanted in a preferential therapy plan that favors energy delivered by the electrodes of lead 4814 instead of leads 4808 because of the desire to spread the electric field strength over at least part of residual tumor 4812. In other examples, one or more leads may be placed directly within residual tumor 4812.

The system may calculate one or more coverage areas that will be possible due to electric field therapy deliverable by medical leads 4808 and 4814. For example, coverage area 4810 may be indicative of the system predicted coverage of electric fields using the preferential treatment plan that directs more power to the electrodes of lead 4814. In other examples, user interface 4801 may display additional coverage options selectable by the user. As shown in the dashed line of coverage 4810, the spread of electric field strength will spread more towards residual tumor 4812.

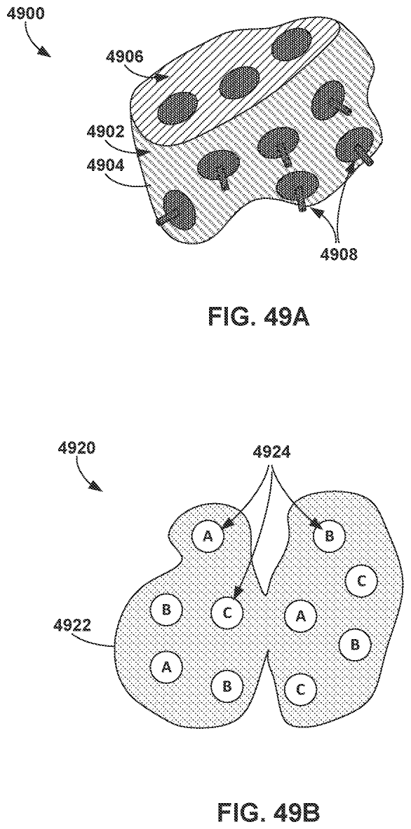

FIG. 49A is a conceptual diagram of example representation of lead configuration 4900 that includes leads 4908 (and corresponding electrodes) implanted within resection bed 4902 surrounding resection void where a tumor was removed. Outer surface 4906 of resection bed 4902 is the surface that faces the void of the resection where the tumor was removed. Inner surface 4904 is a representation of the underside surface of resection bed 4902 in order to display the extension of each lead 4908 into tissue. In some examples, a user interface may display lead configuration 4900 to visualize the implanted electrodes and leads for the patient. Using this representation of lead configuration 4900, the system and/or the user may select stimulation parameters that define which electrodes can be used to deliver stimulation. Lead configuration 4900 may be shown in a three-dimensional state in some examples to assist the user in programming therapy.

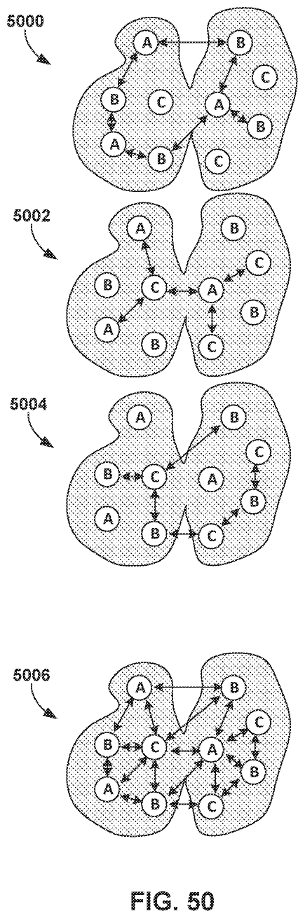

FIG. 49B is a conceptual diagram of an example unwrapped view of the resection bed 4902 and representations 4924 of implanted leads 4908 of FIG. 49A. In order to facilitate visualization of the implanted leads 4908, user interface 4920 may present the unwrapped view of the resection bed by showing bed surface 4922 with representations 4924 that correspond to the location of each respective lead and/or electrodes. This unwrapped view (similar to a Mercator projection) may show a curved surface in a flattened two-dimensional space. In addition to the location of each lead, representations 4924 may also include a group label (e.g., A, B, C, etc.) that indicates which leads are grouped together. This grouping may be physical in that the electrodes of each group are electrically connected to operate together or programmed to operate with a common polarity or signal. In this manner, if electrodes A and C are energized and of opposite polarity, current will flow between electrodes of each group. In other words, the electrodes are wired such that each group is wired together. This is shown in more detail in FIG. 50.

User interface 4920 may provide other user selectable or user definable stimulation parameters, such as amplitude, duty cycle, cycle duration, pulse frequency, or any other parameters. These parameters may be shown to the side of bed surface 4922 or over the respective electrodes.

FIG. 50 is a conceptual diagram of unwrapped views of potential electric fields deliverable via the implanted electrodes of FIG. 49A. One, some, or all of electric field views 5000, 5002, 5004, and 5006 may be presented by the system via a display to visualize the electric fields available for the implanted leads. The clinician or system may select any one or combination of the electric fields in electric field views 5000, 5002, 5004, and 5006 in order to treat the patient. In some examples, the user interface may receive user input defining the duration and/or sequence of one or more of electric field views 5000, 5002, 5004, and 5006 during cycling of the electric field therapy. Electric field view 5000 shows the electric fields between electrodes A and B. Electric field view 5002 shows the electric fields between electrodes A and C. Electric field view 5004 shows the electric fields between electrodes B and C. Electric field view 5006 shows the electric fields between all groups of electrodes A, B, and C which may be delivered simultaneously or on a cycled basis.

In some examples, in addition to, or alternative to, the electric fields between leads, the system may also select a phase in which electric fields are delivered between two or more electrodes on the same lead. This would result in an electric field in the direction of the depth of tissue for medical leads that include two or more electrodes carried on a post of the medical lead such as medical lead 3710.

FIG. 51 is a cross-section of an example medical lead comprising a registration feature 5132 configured to receive a probe for determining the location of medical lead 5110 when implanted within tissue. Medical lead 5110 is an example of tack electrode 3710, but registration feature 5132 may be formed or created on any lead. As shown in the example of FIG. 51, medical lead 5110 includes non-conductive cap 5114 coupled to one or more structures that may include post 5116. Post 5116 is configured to carry one or more electrodes, such as electrodes 5118 and 5120.

Curved surface 5130 in the top surface of non-conductive cap 5114 may be configured to receive the tip of a probe that can convey the location of the probe tip to the system. Using this location and known position of registration feature 5132, the system can determine the location and orientation of lead 5110. Registration feature 5132 is shown as a divot or detent, but may take on other shapes in other examples. For example, the shape and size of registration feature 5132 may be selected to mate with the tip of the probe to the used. In some examples, registration feature 5132 may be offset on lead 5110 in order to determine an orientation of a non-symmetrical lead, or multiple registration features may be used in other examples.

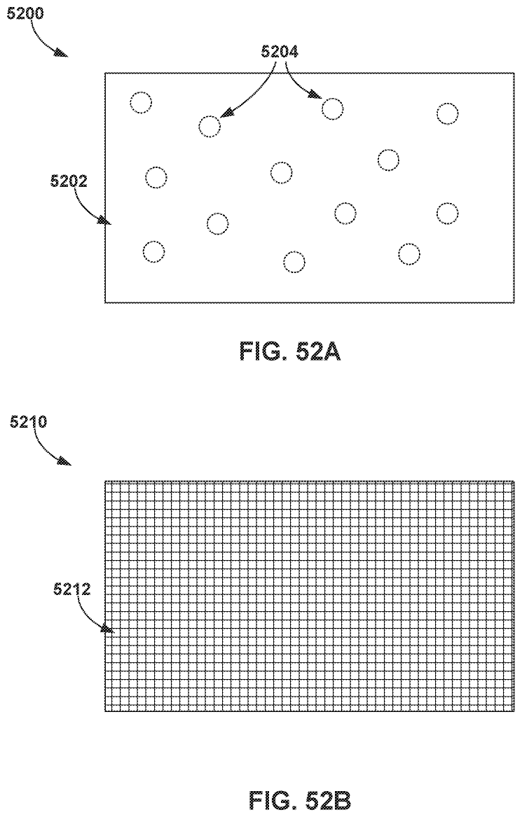

FIGS. 52A and 52B are example implantation guide layers 5200 and 5210 configured to facilitate implantation of leads at target locations of tissue. Each of implantation guide layers 5200 and 5210 may be configured to accept an electrode or post, such as post 5116 of medical lead 5110 through respective positions of the implantation guide layer. In this manner, implantation guide layers 5200 and 5210 may facilitate correct location of lead implantation within the tissue. Implantation guide layers 5200 and 5210 may be created according to the surgical plan in order to identify the target locations of the electrodes or leads. In this manner, implantation guide layers 5200 and 5210 may be used as a “template” for inserting the leads or electrodes into tissue. Implantation guide layers 5200 and 5210 may be laid or pressed against the target tissue (such as a resection bed), and the surgeon can insert the lead through target locations on the layer. Although implantation guide layers 5200 and 5210 are shown as flat materials, implantation guide layers 5200 and 5210 may be flexible to conform to curved tissue surfaces and/or pre-curved to enable an improved placement within the resection cavity. The size and/or shape of implantation guide layers 5200 and 5210 may be formed to fit within the patient and/or cut to size (e.g., excess material may be removed when not needed for insertion into the tissue cavity) by the clinician during the implantation procedure. In some examples, implantation guide layers 5200 and 5210 may be constructed of a biocompatible paper or polymer. Implantation guide layers 5200 and 5210 may be permanent as part of the implanted leads. In some examples, some or all of the implantation guide layer may be constructed of a bioresorbable material that dissolves in the body over time after implantation. In some examples, implantation guide layers 5200 and 5210 may be drug eluting such that the material releases a chemical over time, such as a steroid, chemotherapy compound, fluorescent molecules, radioactive isotope, contrast media, or any other chemical appropriate for therapy, imaging, or promoting success of the implants. For example, implantation guide layers 5200 and 5210 may include a chemical or material that reduces or resists the growth of biofilms.

As shown in the example of FIG. 52A, implantation guide layer 5200 includes a plurality of removable portions 5204 defined by respective perforations within substrate 5202. In this manner, the plurality of removable portions 5204 are configured to be removed from substrate 5202 of implantation guide layer 5200 by separation along the respective perforations. Once removable portions 5204 are removed, implantation guide layer 500 is configured to accept respective electrodes or leads through respective passages formed by removal of the removable portions 5204. The pattern of removable portions 5204 may be random, in a rectangular grid pattern, in a diamond pattern, or in a patient-specific pattern selected based on the patient anatomy. Some or all of removable portions 5204 may remain in place after implantation. In some examples, pressure from the lead may cause the removable portion to separate along the perforations.

As shown in the example of FIG. 52B, implantation guide layer 5210 includes a mesh 5212 of fibers configured to accept the implanted electrodes or leads at respective locations within the implantation guide layer 5210. Mesh 5212 may define passages having a cross-sectional area that facilitate passage of the electrode or lead. In some examples, the fibers may stretch or break to allow passage of the electrode or lead through implantation guide layer 5210.

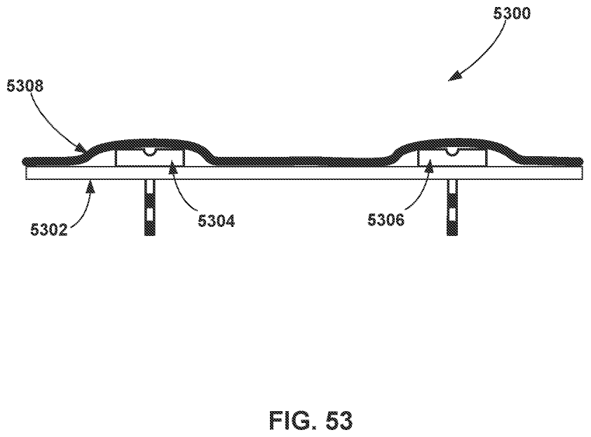

FIG. 53 is a side view of example medical leads 5304 and 5306 inserted through an example implantation guide layer 5302 (such as implantation guide layers 5200 or 5210) secured to the implantation guide layer via an adhesive layer 5308. Adhesive layer 5308 may be configured to secure implanted lead 5304 and 5306 within implantation guide layer 5302. In this manner, after insertion of leads 5304 and 5306 through implantation guide layer 5302, the surgeon can apply adhesive layer 5308 over implantation guide layer 5302 and leads 5304 and 5306 to secure the leads. The adhesive may be biocompatible and/or moisture activated such that adhesive layer 5308 only adheres once in the patient. Although an adhesive is described, other examples may include other types of attachment, such as hook and loop closures, snaps, indent/detents, etc.

The following examples are described herein.

Example 1. A system comprising processing circuitry configured to: receive a request to deliver electric field therapy; determine therapy parameter values that define the electric field therapy, wherein the electric field therapy comprises delivery of a first electric field and a second electric field; control an implantable medical device to deliver the first electric field from a first electrode combination of implanted electrodes; and control the implantable medical device to deliver, alternating with the first electric field, the second electric field from a second electrode combination of implanted electrodes different than the first electrode combination.

Example 2. The system of example 1, wherein the first electrode combination comprises a first set of anodes carried by a first lead; the second electrode combination comprises a first set of cathodes carried by a second lead different than the first lead; the third electrode combination comprises a second set of anodes carried by a third lead different than the first lead and the second lead; and the fourth electrode combination comprises a second set of cathodes carried by a fourth lead different than the first lead, the second lead, and the third lead.

Example 3. The system of any of examples 1 and 2, wherein: the first electrode combination comprises a first set of anodes carried by a first lead; the second electrode combination comprises a first set of cathodes carried by a second lead different than the first lead; the third electrode combination comprises a second set of anodes carried by the second lead; and the fourth electrode combination comprises a second set of cathodes carried by the first lead.

Example 4. The system of any of examples 1 through 3, wherein the processing circuitry is configured to cycle the electric field therapy on and off according to a predetermined schedule.

Example 5. The system of any of examples 1 through 4, wherein the processing circuitry is configured to: receive temperature data indicative of a temperature of tissue that receives the electric field therapy; determine that the temperature exceeds a threshold temperature; and responsive to determining that the temperature exceeds the threshold temperature, terminate delivery of the electric field therapy.

Example 6. The system of any of examples 1 through 5, wherein the processing circuitry is configured to adjust a frequency of the first electric field and the second electric field according to a predetermined schedule.

Example 7. The system of any of examples 1 through 6, wherein the processing circuitry is configured to: receive an indication that a trigger event occurred; and responsive to receiving the indication that the trigger event occurred, adjust a frequency of the first electric field and the second electric field.

Example 8. The system of any of examples 1 through 7, wherein the first electric field is defined by a first frequency, and wherein the second electric field is defined by a second frequency different than the first frequency.

Example 9. The system of any of examples 1 through 8, further comprising a user interface configured to receive user input indicative of target tissue to receive electric field therapy, and wherein the processing circuitry is configured to determine, based on the user input, the first electrode combination and the second electrode combination.

Example 10. The system of any of examples 1 through 9, further comprising a user interface configured to receive user input indicative of tissue to avoid receiving electric field therapy, and wherein the processing circuitry is configured to determine, based on the user input, the first electrode combination and the second electrode combination.

Example 11. The system of any of examples 1 through 10, wherein the processing circuitry is configured to adjust one or more stimulation parameters that at least partially defines the electric field therapy based on histological data obtained from a sample of tissue affected by the electric field therapy.

Example 12. The system of any of examples 1 through 11, wherein the processing circuitry is configured to: determine target tissue for electric field therapy based on water content data obtained from magnetic resonance imaging (MRI) data; and determine, based on the target tissue, the first electrode combination and the second electrode combination for delivery of the electric field therapy.

Example 13. The system of any of examples 1 through 12, wherein the processing circuitry is configured to: determine target tissue for electric field therapy based on impedance tomography data obtained from sensed electrical potentials sensed from two or more of the implanted electrodes; and determine, based on the target tissue, the first electrode combination and the second electrode combination to deliver the electric field therapy.

Example 14. The system of any of examples 1 through 13, wherein the processing circuitry is configured to: generate an electric field dosimetry metric for anatomy that receives the electric field therapy; map the electric field dosimetry across target tissue of the anatomy; and output, for display, the map of the electric field dosimetry with respect to the anatomy.

Example 15. The system of any of examples 1 through 14, wherein the first electrode combination comprises a first set of electrodes defined as cathodes and a second set of electrodes defined as anodes, and wherein the second electrode combination comprises the first set of electrodes defined as anodes and the second set of electrodes defined as cathodes.

Example 16. The system of any of examples 1 through 15, wherein: the first electrode combination comprises a first set of electrodes defined as cathodes and a second set of electrodes defined as anodes; the second electrode combination comprises a third set of electrodes defined as anodes and a fourth set of electrodes defined as cathodes; the third set of electrodes are adjacent to the first set of electrodes in one direction on a first lead; and the fourth set of electrodes are adjacent to the second set of electrodes in the one direction on a second lead.

Example 17. The system of any of examples 1 through 16, wherein the first electrode combination comprises a first set of electrodes defined as cathodes and a second set of electrodes defined as anodes in a first paired configuration from a cube configuration, and wherein the second electrode combination comprises the first set of electrodes defined as anodes and the second set of electrodes defined as cathodes in a second paired configuration from the cube configuration.

Example 18. The system of any of examples 1 through 17, wherein the implanted electrodes are carried by two or more implanted leads.

Example 19. The system of any of examples 1 through 18, wherein at least two electrodes of the implanted electrodes are carried by an electrode array positioned adjacent a resection bed of tissue.

Example 20. The system of any of examples 1 through 19, wherein at least two electrodes of the implanted electrodes are subcutaneous electrodes.

Example 21. The system of any of examples 1 through 20, further comprising a plurality of external cutaneous electrodes, and wherein at least one of the first electrode combination or the second electrode combination comprises one or more electrodes of the plurality of external cutaneous electrodes.

Example 22. The system of any of examples 1 through 21, further comprising the implantable medical device.

Example 23. A computer-readable storage medium comprising instructions that, when executed, cause processing circuitry to: receive a request to deliver electric field therapy; determine therapy parameter values that define the electric field therapy, wherein the electric field therapy comprises delivery of a first electric field and a second electric field; control an implantable medical device to deliver the first electric field from a first electrode combination of implanted electrodes; and control the implantable medical device to deliver, alternating with the first electric field, the second electric field from a second electrode combination of implanted electrodes different than the first electrode combination.

Example 101. A system comprising: processing circuitry configured to: receive imaging data of anatomy for a patient; receive sensing data from one or more implanted sensors in the patient; identify, based on the imaging data and the sensing data, locations of cerebral spinal fluid, a resection cavity, and a possible residual tumor; generate, based on the locations identified, a model of the anatomy for the patient; and output, for display, the model.

Example 102. The system of example 101, wherein the imaging data comprises at least one of data obtained by at least one of magnetic resonance imaging (MRI), computed tomography, or magnetoencephalography (MEG).

Example 103. The system of any of examples 101 and 102, wherein the sensing data comprises at least one of local field potentials (LFPs) or impedance tomography.

Example 104. The system of any of examples 101 through 103, wherein the processing circuitry is configured to generate, as part of the model, locations of high electric field strength.

Example 105. The system of any of examples 101 through 104, wherein the anatomy of the patient comprises at least a portion of a brain of the patient.

Example 106. The system of any of examples 101 through 105, wherein the processing circuitry is configured to generate, as part of the model, anatomical locations within a brain of the patient.

Example 107. The system of any of examples 101 through 106, further comprising a user interface configured to display the model.

Example 108. The system of any of examples 101 through 107, wherein the processing circuitry is configured to determine a value of one or more stimulation parameters that at least partially define alternating electric field therapy.

Example 109. The system of example 108, wherein the processing circuitry is configured to output, for display, the value of the one or more stimulation parameters as a recommendation for alternating electric field therapy as part of a therapy planning user interface.

Example 110. The system of example 109, wherein the value is a first value, and wherein the processing circuitry is configured to: receive user input adjusting the first value of the one or more stimulation parameters to a second value different than the first value; and storing the second value of the one or more stimulation parameters for subsequent delivery of alternating electric field therapy to the patient.

Example 201. A system comprising: stimulation circuitry configured to deliver electric field therapy to a patient; sensing circuitry configured to generate sensed data representative of a sensed electric signal resulting from delivery of the electric field therapy; and processing circuitry configured to: receive the sensed data; determine one or more electrical physics parameters indicative of the sensed electric signal; predict an electrical field strength for anatomy of the patient; generate, based on an electrical field strength, a metric of the electric field therapy; and output, for display, the metric of the electric field therapy.

Example 202. The system of example 201, wherein the stimulation circuitry is configured to deliver the electric field therapy via a first set of electrodes, and wherein the sensing circuitry is configured to generate the sensed data from the sensed electrical signal obtained from a second set of electrodes different than the first set of electrodes.

Example 203. The system of any of examples 201 and 202, wherein the sensing data comprises at least one of evoked signals, local field potentials (LFPs), or impedance tomography.

Example 204. The system of any of examples 201 through 203, wherein the processing circuitry is configured to predict the electric field strength over a volume of the anatomy.

Example 205. The system of any of examples 201 through 204, wherein the anatomy of the patient comprises at least a portion of a brain of the patient.

Example 206. The system of any of examples 201 through 205, wherein the metric comprises a singular value indicative of the electric field therapy efficacy for a target tissue within the anatomy.

Example 207. The system of any of examples 201 through 206, wherein the metric comprises gradients with respect to respective locations of the anatomy of the patient.

Example 208. The system of any of examples 201 through 207, further comprising a user interface configured to display the metric of the electric field therapy.

Example 301. A system comprising: processing circuitry configured to: receive first user input defining target tissue to receive electric field therapy; receive second user input defining tissue to avoid receiving electric field therapy; determine, based on at least one of the first user input or the second user input, one or more stimulation parameters that at least partially defines the electric field therapy; and control a medical device to deliver the electric field therapy according to the one or more stimulation parameters.

Example 302. The system of example 301, wherein the one or more stimulation parameters comprises one or more electrode combinations that at least partially define the electric field therapy.

Example 303. The system of any of examples 301 and 302, wherein the one or more stimulation parameters comprises one or more implant locations for one or more leads that carry electrodes for delivering the electric field therapy.

Example 304. The system of any of examples 301 through 303, further comprising a user interface configured to receive at least one of the first user input or the second user input.

Example 401. A system comprising: processing circuitry configured to: receive a request to deliver electric field therapy, wherein the electric field therapy comprises delivery of a first electric field and a second electric field; and control an implantable medical device to deliver the electric field therapy by iteratively sweeping through each selected frequency of a plurality of frequencies, wherein, for each selected frequency of the plurality of frequencies, the implantable medical device is controlled to deliver the first electric field from a first electrode combination of implanted electrodes at the selected frequency in alternating fashion with the second electric field from a second electrode combination of implanted electrodes different than the first electrode combination and at the selected frequency.

Example 402. The system of example 401, further comprising the implantable medical device configured to deliver the electric field therapy.

Example 403. The system of any of examples 401 and 402, wherein the processing circuitry is configured to determine respective amplitudes for each selected frequency of the plurality of frequencies based on a model of electric field therapy.

Example 501. A system comprising: stimulation circuitry configured to deliver electric field therapy to a patient; sensing circuitry configured to generate sensed data representative of a sensed electric signals via one or more electrodes disposed at a boundary of a tumor resection; and processing circuitry configured to: receive the sensed data; compare the sensed data to one or more target signal characteristics; adjust, based on the comparison, one or more stimulation parameters from a first value to a second value; and control the stimulation circuitry to deliver the electric field therapy according to the second value of the one or more stimulation parameters.

Example 502. The system of example 501, wherein the processing circuitry is configured to compare the sensed data to one or more target signal characteristics at a predetermined interval.

Example 503. The system of any of examples 501 and 502, wherein the one or more stimulation parameters comprises one or more electrode combinations.

Example 504. The system of any of examples 501 through 503, wherein the one or more stimulation parameters comprises a frequency.

Example 505. The system of any of examples 501 through 504, wherein the one or more stimulation parameters comprises a cycling duration for the electric field therapy.

Example 601. A system comprising: processing circuitry configured to: control an implantable medical device to deliver a first electric field from a first electrode combination of implanted electrodes; and control the implantable medical device to deliver, alternating with the first electric field, a second electric field from a second electrode combination of implanted electrodes different than the first electrode combination, wherein the first electric field and the second electric field comprise electric field therapy deliverable to a patient, and wherein at least one of the first electrode combination or the second electrode combination comprises one or more subcutaneous electrodes.

Example 602. The system of example 601, wherein at least one of the first electrode combination or the second electrode combination comprises one or more electrodes carried by one or more implantable leads.

Example 603. The system of any of examples 601 and 602, wherein the implantable medical device is configured to be electrically coupled to electrodes of the first electrode combination and the second electrode combination.

Example 604. The system of any of examples 601 through 603, further comprising the implantable medical device.

Example 605. The system of any of examples 601 through 604, wherein the implantable medical device comprises the processing circuitry.

Example 606. The system of any of examples 601 through 605, further comprising the implanted electrodes that include the one or more subcutaneous electrodes.

Example 701. A system comprising: processing circuitry configured to: control an implantable medical device to deliver a first electric field from a first electrode combination of electrodes carried by one or more implanted leads; and control an external medical device to deliver, alternating with the first electric field, a second electric field from a second electrode combination of external cutaneous electrodes, wherein the first electric field and the second electric field comprise electric field therapy deliverable to a patient.

Example 702. The system of example 701, further comprising the implantable medical device, wherein the implantable medical device comprises the processing circuitry configured to control the external medical device to deliver the second electric field.

Example 703. The system of any of examples 701 and 702, further comprising the external medical device, wherein the external medical device comprises the processing circuitry configured to control the implantable medical device to deliver the first electric field.

Example 704. The system of any of examples 701 through 703, further comprising an external programmer comprising the processing circuitry, wherein the external programmer is configured to wirelessly communicate with the implantable medical device and the external medical device to coordinate delivery of the AEF therapy.

Example 705. The system of any of examples 701 through 704, wherein the implantable medical device comprises the processing circuitry.

Example 706. The system of any of examples 701 through 705, further comprising the implanted leads and the external cutaneous electrodes.

Example 801. A system comprising: an activity sensor configured to generate activity data indicative of patient activity; sensing circuitry configured to sense an electrical signal through at least a portion of target tissue configured to receive electric field therapy; and processing circuitry configured to: receive the activity data; control, based on the activity data, the sensing circuitry to sense the electrical signal; and control, based on the electrical signal, an implantable medical device to deliver electric field therapy.

Example 802. The system of example 801, wherein the processing circuitry is configured to control the sensing circuitry by at least scheduling the sensing circuitry to sense the electrical signal during a period of reduced patient activity.

Example 803. The system of any of examples 801 and 802, wherein the sensing circuitry is configured to sense the electrical signal by sensing an electrical field via two or more implanted electrodes.

Example 804. The system of any of examples 801 through 803, further comprising the implantable medical device.

Example 901. A system comprising: processing circuitry configured to: receive a request to deliver alternating magnetic field (AMF) therapy; determine therapy parameter values that define the AMF therapy, wherein the AMF therapy comprises delivery of a first magnetic field and a second magnetic field; control an implantable medical device to deliver the first magnetic field from at least a first implantable coil; and control the implantable medical device to deliver, alternating with the first magnetic field, the second magnetic field from a second implantable coil different than the first implantable coil.

Example 902. The system of example 901, further comprising the first implanted coil and the second implantable coil.

Example 903. The system of any of examples 901 and 902, further comprising the implantable medical device.

Example 1001. A system comprising: processing circuitry configured to: receive temperature data generated by a temperature sensor, the temperature data indicative of temperature at a tissue region of a patient; control, based on the temperature, an implantable medical device to deliver electric field therapy via a plurality of implantable electrodes.

Example 1002. The system of example 1001, wherein the processing circuitry is configured to: determine, based on the temperature data, that the temperature exceeds a threshold temperature; and responsive to the temperature exceeding the threshold temperature, control the implantable medical device to terminate electric field therapy.

Example 1003. The system of any of examples 1001 and 1002, wherein the processing circuitry is configured to: determine, based on the temperature data, that the temperature drops below an acceptable temperature; and responsive to the temperature dropping below the acceptable temperature, control the implantable medical device to redeliver the electric field therapy.

Example 1004. The system of any of examples 1001 through 1003, further comprising the temperature sensor.

Example 1005. The system of any of examples 1001 through 1004, further comprising the implantable medical device.

Example 1006. The system of any of examples 1001 through 1005, wherein the implantable medical device comprises the processing circuitry.

Example 1101. A system comprising: processing circuitry configured to: receive a request to deliver electric field therapy; determine therapy parameter values that define the electric field therapy, wherein the electric field therapy comprises delivery of a first electric field and a second electric field; control an implantable medical device to deliver the first electric field from a first electrode combination of implanted electrodes; and control the implantable medical device to deliver, alternating with the first electric field, the second electric field from a second electrode combination of implanted electrodes different than the first electrode combination.

Example 1102. The system of example 1101, wherein the implanted electrodes are carried on one or more medical leads, each medical lead of the one or more medical leads comprising: a non-conductive cap; a post coupled to the non-conductive cap and configured to carry one or more electrodes at respective axial positions along the post, and wherein a cross-sectional area of the post is smaller than a cross-sectional area of the non-conductive cap.

Example 1103. The system of example 1102, wherein the one or more medical leads comprise a plurality of medical leads, and wherein each medical lead of the plurality of leads comprises two or more electrodes disposed at different axial positions along the post.

Example 1104. The system of any of examples 1102 and 1103, wherein the post is configured to be inserted into tissue to a depth limited by a length of the post extending from a distal surface of the non-conductive cap.

Example 1105. The system of any of examples 1102 through 1104, wherein the one or more medical leads comprises a first medical lead comprising a first post having a first length and a second medical lead comprising a second post having a second length different than the first length.

Example 1106. The system of any of examples 1101 through 1105, wherein each medical lead of the one or more medial leads comprises a respective conductor extending from the respective non-conductive cap.

Example 1107. The system of any of examples 1101 through 1106, wherein each electrode of the implanted electrodes is associated with the respective conductor.

Example 1108. The system of any of examples 1101 through 1107, further comprising the one or more medical leads, and wherein the one or more medical leads are configured to position the plurality of electrodes within tissue with respect to a tissue resection region.

Example 1109. The system of any of examples 1101 through 1108, wherein the processing circuitry is configured to: estimate propagation of the first electric field and the second electric field through tissue; and cycle, based on the estimate, the first electric field and the second electric field on and off.

Example 1110. The system of any of examples 1101 through 1109, further comprising an implantation guide layer configured to accept the implanted electrodes through respective positions of the implantation guide layer.

Example 1111. The system of example 1110, wherein the implantation guide layer comprises a plurality of removable portions defined by respective perforations, and wherein the plurality of removable portions are configured to be removed from the implantation guide layer by separation along the respective perforations, and wherein the implantation guide layer is configured to accept respective electrodes of the implanted electrodes through respective passages formed by removal of the removable portions.

Example 1112. The system of example 1110, wherein the implantation guide layer comprises a mesh of fibers configured to accept the implanted electrodes at respective locations within the implantation guide layer.

Example 1113. The system of any of examples 1110 through 1112, wherein the implantation guide layer bioresorbable.

Example 1114. The system of any of examples 1110 through 1113, further comprising an adhesive layer configured to secure the implanted electrodes within the implantation guide layer.

Example 1115. The system of any of examples 1101 through 1114, further comprising the implantable medical device comprising the processing circuitry and stimulation circuitry configured to deliver the first electric field and the second electric field.

A variety of different therapies are described herein that are related to each other, but some are slightly different. Generally, electric and magnetic stimulation therapy covers all of the therapies described herein. This includes, for example, direct current stimulation (DCS). Electric field therapy includes alternating current stimulation, which also includes alternating electric field (AEF) therapy, which includes tumor treating field (TTF) therapy (e.g., AEF therapy within a range of 100 kHz to 500 kHz). Electric field therapy also includes pulse electric fields, which includes nanosecond pulsed electric fields (or nanopulse stimulation), which includes both irreversible electroporation and reversible electroporation. Electric and magnetic stimulation therapy also includes magnetic field therapy, which includes examples such as alternating magnetic field (AMF) therapy, oscillating magnetic field (OMF) therapy, and extremely low frequency electromagnetic field (ELF-EMF) therapy. Other types of therapy may also be included within any of these example categories of therapies.

The techniques described in this disclosure may be implemented, at least in part, in hardware, software, firmware or any combination thereof. For example, various aspects of the described techniques may be implemented within one or more processors, such as fixed function processing circuitry and/or programmable processing circuitry, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit comprising hardware may also perform one or more of the techniques of this disclosure.

Such hardware, software, and firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

The techniques described in this disclosure may also be embodied or encoded in a computer-readable medium, such as a computer-readable storage medium, containing instructions. Instructions embedded or encoded in a computer-readable storage medium may cause a programmable processor, or other processor, to perform the method, e.g., when the instructions are executed. Computer readable storage media may include random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer readable media.

Various examples have been described. These and other examples are within the scope of the following claims.

1. A system comprising:
processing circuitry configured to:
receive a request to deliver electric field therapy;
determine therapy parameter values that define the electric field therapy, wherein the electric field therapy comprises alternating delivery of a first electric field from a first electrode combination of implanted electrodes within a cranium of the patient and a second electric field from a second electrode combination of the implanted electrodes different from the first electrode combination;
control an implantable medical device implanted within the patient to deliver the first electric field from the first electrode combination of the implanted electrodes; and
control the implantable medical device to deliver, alternating with the first electric field, the second electric field from the second electrode combination of the implanted electrodes.

2. The system of claim 1, wherein the implanted electrodes are carried on one or more medical leads, each medical lead of the one or more medical leads comprising:
a non-conductive cap;
a post coupled to the non-conductive cap and configured to carry one or more electrodes at respective axial positions along the post, and wherein a cross-sectional area of the post is smaller than a cross-sectional area of the non-conductive cap.

3. The system of claim 2, wherein the one or more medical leads comprise a plurality of medical leads, and wherein each medical lead of the plurality of leads comprises two or more electrodes disposed at different axial positions along the post.

4. The system of claim 2, wherein the post is configured to be inserted into tissue to a depth limited by a length of the post extending from a distal surface of the non-conductive cap.

5. The system of claim 2, wherein the one or more medical leads comprises a first medical lead comprising a first post having a first length and a second medical lead comprising a second post having a second length different than the first length.

6. The system of claim 1, wherein each medical lead of the one or more medial leads comprises a respective conductor extending from the respective non-conductive cap.

7. The system of claim 1, wherein each electrode of the implanted electrodes is associated with the respective conductor.

8. The system of claim 1, further comprising the one or more medical leads, and wherein the one or more medical leads are configured to position the plurality of electrodes within tissue with respect to a tissue resection region.

9. The system of claim 1, wherein the processing circuitry is configured to:
estimate propagation of the first electric field and the second electric field through tissue; and
cycle, based on the estimate, the first electric field and the second electric field on and off.

10. The system of claim 1, further comprising an implantation guide layer configured to accept the implanted electrodes through respective positions of the implantation guide layer.

11. The system of claim 10, wherein the implantation guide layer comprises a plurality of removable portions defined by respective perforations, and wherein the plurality of removable portions are configured to be removed from the implantation guide layer by separation along the respective perforations, and wherein the implantation guide layer is configured to accept respective electrodes of the implanted electrodes through respective passages formed by removal of the removable portions.

12. The system of claim 10, wherein the implantation guide layer comprises a mesh of fibers configured to accept the implanted electrodes at respective locations within the implantation guide layer.

13. The system of claim 10, wherein the implantation guide layer bioresorbable.

14. The system of claim 10, further comprising an adhesive layer configured to secure the implanted electrodes within the implantation guide layer.

15. The system of claim 1, further comprising the implantable medical device comprising the processing circuitry and stimulation circuitry configured to deliver the first electric field and the second electric field.

16. A method comprising:
receiving, by processing circuitry, a request to deliver electric field therapy;
determining, by the processing circuitry, therapy parameter values that define the electric field therapy, wherein the electric field therapy comprises alternating delivery of a first electric field from a first electrode combination of implanted electrodes within a cranium of the patient and a second electric field from a second electrode combination of the implanted electrodes different from the first electrode combination;
controlling, by the processing circuitry, an implantable medical device implanted within the patient to deliver the first electric field from the first electrode combination of the implanted electrodes; and
controlling, by the processing circuitry, the implantable medical device to deliver, alternating with the first electric field, the second electric field from the second electrode combination of the implanted electrodes.

17. The method of claim 16, wherein the implanted electrodes are carried on one or more medical leads, each medical lead of the one or more medical leads comprising:
a non-conductive cap;
a post coupled to the non-conductive cap and configured to carry one or more electrodes at respective axial positions along the post, and wherein a cross-sectional area of the post is smaller than a cross-sectional area of the non-conductive cap.

18. The method of claim 17, wherein
the one or more medical leads comprise a plurality of medical leads;
each medical lead of the plurality of leads comprises two or more electrodes disposed at different axial positions along the post; and
the post is configured to be inserted into tissue to a depth limited by a length of the post extending from a distal surface of the non-conductive cap.

19. The method of claim 16, further comprising:
estimating, by the processing circuitry, propagation of the first electric field and the second electric field through tissue; and
cycling, by the processing circuitry and based on the estimated propagation, the first electric field and the second electric field on and off.

20. A computer-readable storage medium comprising instructions that, when executed, cause processing circuitry to:
receive a request to deliver electric field therapy;
determine therapy parameter values that define the electric field therapy, wherein the electric field therapy comprises alternating delivery of a first electric field from a first electrode combination of implanted electrodes within a cranium of the patient and a second electric field from a second electrode combination of the implanted electrodes different from the first electrode combination;
control an implantable medical device implanted within the patient to deliver the first electric field from the first electrode combination of the implanted electrodes; and
control the implantable medical device to deliver, alternating with the first electric field, the second electric field from the second electrode combination of the implanted electrodes.
